SEQUENCE LISTING (35 sequences)

SEQ ID NO: 1 - DNA, 24 bp, Artificial Sequence
  Synthetic Construct
gttaagagcgtcttccttgtttag

SEQ ID NO: 2 - DNA, 24 bp, Artificial Sequence
  Synthetic Construct
gtgacgacttaaatgtggagtatc

SEQ ID NO: 3 - DNA, 24 bp, Artificial Sequence
  Synthetic Construct
atggtcattacgacgaggataagc

SEQ ID NO: 4 - DNA, 24 bp, Artificial Sequence
  Synthetic Construct
gtcaatgactagtttcgagtatag

SEQ ID NO: 5 - DNA, 24 bp, Artificial Sequence
  Synthetic Construct
gcggcaacgagcaaatatgcgtat

SEQ ID NO: 6 - DNA, 24 bp, Artificial Sequence
  Synthetic Construct
gggacgatccaagactcatcagag

SEQ ID NO: 7 - DNA, 24 bp, Artificial Sequence
  Synthetic Construct
ttgttcatcgaggtaaggtcaggc

SEQ ID NO: 8 - DNA, 24 bp, Artificial Sequence
  Synthetic Construct
cgcagctaagatcggagagacgta

SEQ ID NO: 9 - DNA, 24 bp, Artificial Sequence
  Synthetic Construct
aacccgagatcgtagtatactcaa

SEQ ID NO: 10 - DNA, 24 bp, Artificial Sequence
  Synthetic Construct
tcaccaaagctcggtcacttgttg

SEQ ID NO: 11 - DNA, 24 bp, Artificial Sequence
  Synthetic Construct
gcgatcgcgtttagttgtatttct

SEQ ID NO: 12 - DNA, 24 bp, Artificial Sequence
  Synthetic Construct
tttatcgaataagtctaatgctct

SEQ ID NO: 13 - DNA, 24 bp, Artificial Sequence
  Synthetic Construct
tacctctatccccaacgtgcaaca

SEQ ID NO: 14 - DNA, 24 bp, Artificial Sequence
  Synthetic Construct
atgtccatccgttttgccatatga

SEQ ID NO: 15 - DNA, 24 bp, Artificial Sequence
  Synthetic Construct
ctatagcctccgtggataaactgg

SEQ ID NO: 16 - DNA, 24 bp, Artificial Sequence
  Synthetic Construct
ctaaacaaggaagacgctcttaac

SEQ ID NO: 17 - DNA, 24 bp, Artificial Sequence
  Synthetic Construct
gatactccacatttaagtcgtcac

SEQ ID NO: 18 - DNA, 24 bp, Artificial Sequence
  Synthetic Construct
gcttatcctcgtcgtaatgaccat

SEQ ID NO: 19 - DNA, 24 bp, Artificial Sequence
  Synthetic Construct
ctatactcgaaactagtcattgac

SEQ ID NO: 20 - DNA, 24 bp, Artificial Sequence
  Synthetic Construct
atacgcatatttgctcgttgccgc

SEQ ID NO: 21 - DNA, 24 bp, Artificial Sequence
  Synthetic Construct
ctctgatgagtcttggatcgtccc

SEQ ID NO: 22 - DNA, 24 bp, Artificial Sequence
  Synthetic Construct
gcctgaccttacctcgatgaacaa

SEQ ID NO: 23 - DNA, 24 bp, Artificial Sequence
  Synthetic Construct
tacgtctctccgatcttagctgcg

SEQ ID NO: 24 - DNA, 24 bp, Artificial Sequence
  Synthetic Construct
ttgagtatactacgatctcgggtt

SEQ ID NO: 25 - DNA, 24 bp, Artificial Sequence
  Synthetic Construct
caacaagtgaccgagctttggtga

SEQ ID NO: 26 - DNA, 24 bp, Artificial Sequence
  Synthetic Construct
agaaatacaactaaacgcgatcgc

SEQ ID NO: 27 - DNA, 24 bp, Artificial Sequence
  Synthetic Construct
agagcattagacttattcgataaa

SEQ ID NO: 28 - DNA, 24 bp, Artificial Sequence
  Synthetic Construct
tgttgcacgttggggatagaggta

SEQ ID NO: 29 - DNA, 24 bp, Artificial Sequence
  Synthetic Construct
tcatatggcaaaacggatggacat

SEQ ID NO: 30 - DNA, 24 bp, Artificial Sequence
  Synthetic Construct
ccagtttatccacggaggctatag

SEQ ID NO: 31 - DNA, 25 bp, Artificial Sequence
  Synthetic Construct
attcctaggacccctgctcgtgtta

SEQ ID NO: 32 - DNA, 31 bp, Artificial Sequence
  Synthetic Construct
ccccctagaaaattgagagaagtccaccacg

SEQ ID NO: 33 - DNA, 110 bp, Artificial Sequence
  Synthetic Construct
ttcctaggacccctgctcgtgttacaggcggggtttttcttgttgacaagaatcctcacaataccgcagagtctagactcgtggtggacttctctcaattttctaggggg

SEQ ID NO: 34 - DNA, 26 bp, Artificial Sequence
  Synthetic Construct
ctcacatccaacaatacaggtcacat

SEQ ID NO: 35 - DNA, 24 bp, Artificial Sequence
  Synthetic Construct
catgagaaatatcacagtaattgg

(19) United States
(12) Patent Application Publication
(10) Pub. No.: US 2020/0385792 A1
(43) Pub. Date: Dec. 10, 2020

(54) SELF-ASSEMBLING DIAGNOSTIC ARRAY PLATFORM

(71) Applicant: Quotient Suisse SA, Eysins (CH)

(72) Inventors: Robert BOHANNON, Elkhart, IN (US); Mark STUART, Elkhart, IN (US); David ROBSON, Eysins (CH); Edward FARRELL, Eysins (CH); Lynda HENDERSON, Eysins (CH); Nathan MCOWEN, Elkhart, IN (US); Seven BOHANNON, Elkhart, IN (US)

(73) Assignee: Quotient Suisse SA, Eysins (CH)

(21) Appl. No.: 16/959,583

(22) PCT Filed: Dec. 19, 2018
(86) PCT No.: PCT/EP2018/085945

§ 371 Date: Jul. 1, 2020

Related U.S. Application Data
(60) Provisional application No. 62/614,313, filed on Jan. 5, 2018.

(51) Int. Cl. C12Q 1/6837; B01L 7/00; C12Q 1/6853

(52) U.S. Cl. CPC C12Q 1/6837; C12Q 1/6853; B01L 7/525

(57) ABSTRACT

The present disclosure relates to methods and kits for detecting a nucleic acid or antigen in a sample using a universal array platform. For example, a nucleic acid can be detected by amplifying at least a portion of a nucleic acid from a sample using a primer pair comprising a first primer comprising a label and a first oligonucleotide sequence that hybridizes with a first strand of the portion of the nucleic acid and a second primer comprising a second oligonucleotide sequence that hybridizes with a second strand of the portion of the nucleic acid; contacting the amplicon, if present, to a plurality of single-stranded oligonucleotide capture sequences each affixed to a solid support; applying a colloidal detection reagent to the solid supports; washing the solid supports with a wash solution; and detecting the colloidal detection reagent. The present disclosure further relates to specific capture and tether oligonucleotide sequences.

### CROSS REFERENCE TO RELATED APPLICATIONS

This application claims the priority benefit of U.S. Provisional Application Ser. No. 62/614,313, filed Jan. 5, 2018, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 709252000140SEQLIST.txt, date recorded: Dec. 14, 2018, size: 7 KB).

### FIELD

The present disclosure relates to methods and kits for detecting nucleic acids, antigens, or both in a sample using a universal array platform and to an apparatus for, and methods of, amplification of nucleic acids comprising at least three stationary temperature zones.

### BACKGROUND

Microarray technology has been adapted for the detection of a wide array of nucleic acids, proteins, and other antigens, particularly in the field of nucleic acid testing (NAT). The existing micro-array format requires printing different capture reagents (e.g., antibodies or single-stranded oligonucleotides) against targets on an activated slide, usually in duplicate or triplicate, along with control spots. When testing a sample for the presence of an antigen of interest, the sample reacts with the array whereby the target is captured, sample washed off, a labeled antibody is used to detect captured targets, and amplification/detection methods to visualize the reaction, and recorded using a reader. This multi-step process takes time and the costs of printing many different capture spots increases dramatically when testing various panels. In addition, each array created must be manufactured and quality checked for each target group AND each target type (e.g., antibody, antigen, nucleic acid, etc.), thereby requiring the manufacture and inventory of multiple array types, which drive costs up.

Microarrays are created by attaching capture ligands onto a solid surface. With increasing ability to spot smaller and smaller spots more accurately, these microarrays can detect a few targets to millions of targets depending upon the density, spot size, etc. On a normal array, each spot, or series of spots, contain a capture oligonucleotide complementary to the target, and antigen whereby an antibody in the sample binds the affixed target, or an antibody is printed onto the array (affixed) to bind targets in the sample, which are subsequently labeled and detected, although some arrays utilize non-labeled targets as well. The creation of these arrays becomes very cumbersome and expensive to produce since each spot is a different material and potentially hundreds, thousands, or even millions of different capture ligands are required to produce a single array.

The detection of infectious agents, biomarkers, toxins, and cells in human clinical samples is paramount for disease-free transfusions and transplants, as well as a variety of diagnostic purposes. However, as described supra, time and cost for manufacturing different microarray slides for different agents, biomarkers, polynucleotides, antigens, etc. can be prohibitive. There is a need for microarray platform technology that allows for the robust and accurate detection of a variety of nucleic acids or antigens in a sample while reducing associated manufacturing costs.

### BRIEF SUMMARY

To meet these and other demands, described herein is a “universal array” approach to microarray platform technology. Such “microarrays” include any platform capable of multiplex detection including, without limitation, planar microarrays, strips, threads, beads, and well arrays. Using this approach, a single type of array can be printed, e.g., with an array of oligonucleotide spots, each conjugated to the solid support (e.g., via a spacer reagent such as bovine serum albumin, BSA). This universal array can be adapted for detection of a wide variety of nucleic acids in a sample by amplification using a primer with an adaptor oligonucleotide sequence that allows the resultant PCR product to hybridize with an oligonucleotide sequence spotted on the array. Each primer pair for a nucleic acid sequence of interest can contain one of these unique adaptor sequences, allowing each amplicon to hybridize to a different spot on the array. In this way, a common or “universal” microarray slide can be adapted to detection of a multitude of different nucleic acids. In addition, the universal array approach can also be adapted to antigens such as polypeptides using specific antibodies conjugated to an adaptor oligonucleotide sequence that hybridizes to a corresponding oligonucleotide sequence spotted on the array. Thus, manufacturing costs are significantly minimized, as a single microarray can be produced and adapted for a variety of different nucleic acid or antigen detection assays.

Accordingly, certain aspects of the present disclosure provide methods for detecting a nucleic acid in a sample. In some embodiments, the method includes: a) amplifying at least a portion of a nucleic acid from a sample using a primer pair under conditions suitable for amplification of an amplicon comprising the portion of the nucleic acid if present in the sample, wherein the primer pair comprises: 1) a first primer comprising a label and a first oligonucleotide sequence that hybridizes with a first strand of the portion of the nucleic acid, and 2) a second primer comprising a second oligonucleotide sequence that hybridizes with a second strand of the portion of the nucleic acid opposite the first strand and a third oligonucleotide sequence; b) after step (a), contacting the amplicon, if present, to a plurality of single-stranded oligonucleotide capture sequences each affixed to a solid support, and wherein the amplicon, if present, hybridizes with at least one of the single-stranded oligonucleotide capture sequences on its solid support via the third oligonucleotide sequence or the complement of the third oligonucleotide sequence; c) after step (a), applying a colloidal detection reagent to the solid supports, wherein the colloidal detection reagent comprises a first moiety that binds to the label of the amplicon if present and a second moiety that comprises a colloidal metal; d) after (c), washing the solid supports with a wash solution; and e) after steps (a)-(d), detecting the colloidal detection reagent, wherein detection of the colloidal detection reagent on a solid support indicates the presence of the hybridized amplicon, thereby detecting the nucleic acid in the sample. In some embodiments, the solid supports are arranged as a microarray, a multiplex bead array, or a well array. In some embodiments, the solid supports are nitrocellulose, silica, plastic, or hydrogel. In some embodiments, detecting the colloidal detection reagent in step (e) comprises detection of the colloidal metal. In some embodiments, detecting the colloidal detection reagent in step (e) comprises: 1) applying a developing reagent to the solid supports, wherein the developing agent is suitable for forming a precipitate in the presence of the colloidal metal; and 2) detecting the colloidal detection reagent by detecting the formation of the precipitate on a solid support. In some embodiments, the formation of the precipitate is detected by visual, electronic, or magnetic detection. In some embodiments, the formation of the precipitate is detected by a mechanical reader. In some embodiments, the developing reagent comprises silver. In some embodiments, the conditions in step (a) are suitable for amplification by polymerase chain reaction (PCR). In some embodiments, the conditions in step (a) are suitable for amplification by recombinase-polymerase assay (RPA), nucleic acid sequenced-based chain assay (NASBA), rolling circle amplification, branched chain amplification, ligation amplification, or loop-mediated isothermal amplification. In some embodiments, the label comprises biotin and the third oligonucleotide sequence hybridizes with at least one of the single-stranded oligonucleotide capture sequences. In some embodiments, each single-stranded oligonucleotide capture sequence is coupled to a spacer reagent, and the spacer reagent is coupled to the corresponding solid support. In some embodiments, the spacer reagent comprises a serum albumin protein. In some embodiments, the spacer reagent comprises a dendrimer. In some embodiments, the method further comprises washing the solid supports with a wash solution after step (b). In some embodiments, the first primer is a forward primer that amplifies in the sense direction of the nucleic acid, and the second primer is a reverse primer that amplifies in the antisense direction of the nucleic acid. In some embodiments, the second primer is a forward primer that amplifies in the sense direction of the nucleic acid, and the first primer is a reverse primer that amplifies in the antisense direction of the nucleic acid. In some embodiments, the second primer comprises: the second oligonucleotide sequence, wherein the second oligonucleotide sequence allows for primer extension in the 5′ to 3′ direction; and the third oligonucleotide sequence, wherein the third oligonucleotide sequence is oriented in the opposite 5′ to 3′ direction compared with the direction of primer extension from the second oligonucleotide sequence. In some embodiments, the third oligonucleotide sequence comprises a modified nucleotide at the 3′ terminus that blocks primer extension. In some embodiments, the second primer further comprises one or more linkers between the 5′ end of the third oligonucleotide sequence and the 5′ end of the second oligonucleotide sequence. In some embodiments, the portion of the nucleic acid is amplified in step (a) using an excess of the first primer relative to the second primer, and wherein the amplicon, if present, is a single-stranded nucleic acid that hybridizes with at least one of the single-stranded oligonucleotide capture sequences in step (b) via the complement of the third oligonucleotide sequence. In some embodiments, the portion of the nucleic acid is amplified in step (a) using a ratio of first primer to the second primer of between about 12.5:1 and about 100:1. In some embodiments, the label of the first primer comprises biotin. In some embodiments, the first moiety of the colloidal detection reagent comprises neutravidin, streptavidin, or an antigen-binding domain that specifically binds biotin. In some embodiments, the first moiety of the colloidal detection reagent comprises neutravidin, and wherein the second moiety of the colloidal detection reagent comprises a colloidal gold ion. In some embodiments, the colloidal detection reagent is applied to the solid supports in step (c) at a final dilution of 0.00001OD to 20OD. In some embodiments, the first moiety of the colloidal detection reagent comprises neutravidin, wherein the second moiety of the colloidal detection reagent comprises a colloidal gold ion, and wherein the colloidal detection reagent is applied to the solid supports in step (c) at a final dilution of 0.05OD to 0.2OD. In some embodiments, 1 pL to 1000 μL of colloidal detection reagent is applied to the solid supports in step (c) per μL of amplicon. In some embodiments, the method further comprises, prior to step (a), exposing the sample to a lysis buffer comprising greater than or equal to 0.1% and less than or equal to 10% N,N-dimethyl-N-dodecylglycine betaine (w/v). In some embodiments, the lysis buffer comprises greater than or equal to 0.5% and less than or equal to 4% N,N-dimethyl-N-dodecylglycine betaine (w/v). In some embodiments, the lysis buffer comprises greater than or equal to 1% and less than or equal to 2% N,N-dimethyl-N-dodecylglycine betaine (w/v). In some embodiments, the sample is exposed to the lysis buffer at a ratio between 1:50 sample:lysis buffer and 50:1 sample:lysis. In some embodiments, the portion of the sample is exposed to the lysis buffer at a ratio of about 1:1 sample:lysis buffer. In some embodiments, the lysis buffer further comprises 0.1× to 5× phosphate buffered saline (PBS) buffer or Tris EDTA (TE) buffer. In some embodiments, the lysis buffer further comprises 1×PBS. In some embodiments, the amplicon is hybridized to the solid supports in step (b) in a hybridization buffer comprising 0.1× to 10× saline sodium citrate (SSC) buffer, 0.001% to 30% blocking agent, and 0.01% to 30% crowding agent. In some embodiments, the blocking agent comprises bovine serum albumin (BSA), polyethylene glycol (PEG), casein, or polyvinyl alcohol (PVA). In some embodiments, the blocking agent comprises BSA, and the BSA is present in the hybridization buffer at 1% to 3%. In some embodiments, the crowding agent is Polyethylene Glycol Bisphenol A Epichlorohydrin Copolymer. In some embodiments, the Polyethylene Glycol Bisphenol A Epichlorohydrin Copolymer is present in the hybridization buffer at 1% to 3%. In some embodiments, the hybridization buffer comprises 2× to 5×SSC buffer. In some embodiments, the method further comprises, prior to step (b), blocking the solid supports using a solution comprising BSA. In some embodiments, the solid supports are blocked for 1 hour at 37° C. using 2% BSA solution. In some embodiments, the method further comprises washing the solid supports with a wash solution after blocking the solid supports. In some embodiments, the method further comprises, after step (b) and prior to step (c), washing the solid supports with a wash buffer comprising 0.1× to 10×SSC buffer and 0.01% to 30% detergent. In some embodiments, the detergent comprises 0.05% to 5% N-lauroylsarcosine sodium salt. In some embodiments, the wash buffer comprises 1× to 5×SSC buffer. In some embodiments, one or more of the lysis buffer, wash buffer, and hybridization buffer further comprises a control oligonucleotide that hybridizes with at least one of the single-stranded oligonucleotide capture sequences on its solid support. In some embodiments, the method further comprises, prior to step (a): (i) contacting the sample with an oligonucleotide coupled to a solid substrate, wherein the oligonucleotide hybridizes with the nucleic acid if present in the sample; (ii) washing the solid substrate under conditions suitable to remove non-specific interactions with the solid substrate but retain the nucleic acid hybridized with the oligonucleotide, if present in the sample; and (iii) eluting the nucleic acid, if present in the sample, from the oligonucleotide, wherein the eluted nucleic acid is subjected to PCR amplification in step (a). In some embodiments, the method further comprises, prior to step (a): (i) contacting the sample with an oligonucleotide, wherein the oligonucleotide hybridizes with the nucleic acid if present in the sample, (ii) simultaneous with or after step (i), contacting the sample with a solid substrate, wherein the solid substrate is coupled to a first binding moiety, wherein the oligonucleotide is coupled to a second binding moiety that binds the first binding moiety, and wherein the sample is contacted with the solid substrate under conditions suitable for the second binding moiety to bind the first binding moiety; (iii) washing the solid substrate under conditions suitable to remove non-specific interactions with the solid substrate but retain the oligonucleotide and the nucleic acid hybridized with the oligonucleotide, if present in the sample; and (iv) eluting the nucleic acid, if present in the sample, from the oligonucleotide, wherein the eluted nucleic acid is subjected to PCR amplification in step (a). In some embodiments, the oligonucleotide is coupled to the solid substrate via a covalent interaction. In some embodiments, the oligonucleotide is coupled to the solid substrate via an avidin:biotin or streptavidin:biotin interaction, or wherein the first binding moiety comprises avidin, neutravidin, streptavidin, or a derivative thereof and the second binding moiety comprises biotin or a derivative thereof. In some embodiments, the solid substrate is positioned in a pipet tip, and wherein step (i) comprises pipetting the sample into the pipet tip. In some embodiments, the solid substrate comprises a matrix or plurality of beads. In some embodiments, the nucleic acid comprises DNA. In some embodiments, the nucleic acid comprises RNA. In some embodiments, the method further comprises, prior to step (a), incubating at least a portion of the sample with a reverse transcriptase, primers, and deoxyribonucleotides under conditions suitable for generation of a cDNA synthesized from the nucleic acid, wherein the portion of the nucleic acid is amplified in step (a) using the cDNA. In some embodiments, the primers used prior to step (a) are random primers, poly-dT primers, or primers specific for the portion of the nucleic acid. In some embodiments, the portion of the sample is incubated with the reverse transcriptase, primers, and the deoxyribonucleotides in the presence of an RNase inhibitor. In some embodiments, the portion of the sample is incubated with the reverse transcriptase, primers, and the deoxyribonucleotides in the presence of betaine. In some embodiments, the betaine is present at a concentration of about 0.2M to about 1.5M. In some embodiments, the nucleic acid comprises a viral nucleic acid. In some embodiments, the viral nucleic acid is from a virus selected from the group consisting of HIV, HBV, HCV, West Nile, Zika, and parvovirus. In some embodiments, the nucleic acid comprises a bacterial, archaean, protozoan, fungal, plant, or animal nucleic acid.

Other aspects of the present disclosure relate to kits or articles of manufacture for detecting a nucleic acid in a sample. In some embodiments, the present disclosure relates to a kit, comprising: a plurality of primer pairs, wherein each primer pair of the plurality comprises a first primer coupled to a label, wherein the first primer hybridizes with a first strand of a nucleic acid, and a second primer comprising: 1) a first oligonucleotide sequence that allows for primer extension in the 5′ to 3′ direction and hybridizes with a second strand of the nucleic acid opposite the first strand; 2) a second oligonucleotide sequence, wherein the second oligonucleotide sequence is oriented in the opposite 5′ to 3′ direction compared with the direction of primer extension from the second oligonucleotide sequence; and 3) one or more linkers between the 5′ end of the first oligonucleotide sequence and the 5′ end of the second oligonucleotide sequence. In some embodiments, the second oligonucleotide sequence comprises a modified nucleotide at the 3′ terminus that blocks primer extension. In some embodiments, the label coupled to the first primer comprises biotin. In some embodiments, the kit further comprises a plurality of single-stranded oligonucleotide capture sequences each affixed to a solid support, and wherein at least one single-stranded oligonucleotide sequence on its solid support hybridizes with the second oligonucleotide sequence of a second primer of a primer pair of the plurality. In some embodiments, the present disclosure relates to a kit, comprising: a) a plurality of single-stranded oligonucleotide capture sequences each affixed to a solid support; and b) a plurality of primer pairs, wherein each primer pair of the plurality comprises: 1) a first primer comprising a label and a first oligonucleotide sequence that hybridizes with a first strand of the portion of the nucleic acid, and 2) a second primer comprising a second oligonucleotide sequence that hybridizes with a second strand of the portion of the nucleic acid opposite the first strand and a third oligonucleotide sequence, wherein the third oligonucleotide sequence of each primer pair of the plurality hybridizes with a single-stranded oligonucleotide capture sequence on its solid support. In some embodiments, the second oligonucleotide sequence of each primer pair of the plurality allows for primer extension in the 5′ to 3′ direction, wherein the third oligonucleotide sequence of each primer pair of the plurality is oriented in the opposite 5′ to 3′ direction compared with the direction of primer extension from the second oligonucleotide sequence, and wherein the second primer of each primer pair of the plurality further comprises one or more linkers between the 5′ end of the third oligonucleotide sequence and the 5′ end of the second oligonucleotide sequence. In some embodiments, the third oligonucleotide sequence of each primer pair of the plurality comprises a modified nucleotide at the 3′ terminus that blocks primer extension. In some embodiments, each of the single-stranded oligonucleotide capture sequences on its support is coupled to a spacer reagent, and the spacer reagent is coupled to the solid support. In some embodiments, the spacer reagent comprises a serum albumin protein. In some embodiments, the spacer reagent comprises a dendrimer.

Certain other aspects of the present disclosure relate to methods for amplifying and detecting a nucleic acid in a sample. In some embodiments, the method comprises: a) incubating at least a portion of the sample with an amplification mixture comprising deoxyribonucleotides, a polymerase, and a primer pair, wherein the primer pair comprises a first primer comprising a label and a first oligonucleotide sequence that hybridizes with a first strand of a portion of the nucleic acid, and a second primer comprising a second oligonucleotide sequence that hybridizes with a second strand of the portion of the nucleic acid opposite the first strand and a first capture moiety; b) passing the portion of the sample in admixture with the amplification mixture through first, second, and third stationary temperature zones for a plurality of cycles through continuous capillary tubing under conditions suitable for amplification of an amplicon comprising the portion of the nucleic acid, if present in the sample, wherein each cycle of the plurality comprises: 1) passing the portion of the sample in admixture with the amplification mixture through the first stationary temperature zone via the continuous capillary tubing at a first temperature and for a first duration suitable for denaturing the strands of the nucleic acid, if present in the sample, 2) after step (b)(1), passing the portion of the sample in admixture with the amplification mixture through the second stationary temperature zone via the continuous capillary tubing at a second temperature and for a second duration suitable for annealing the first and second primers to the respective strands of the nucleic acid, if present in the sample, and 3) after step (b)(2), passing the portion of the sample in admixture with the amplification mixture through the third stationary temperature zone via the continuous capillary tubing at a third temperature and for a third duration suitable for amplifying the nucleic acid target, if present in the sample, via the polymerase and primer pair; c) after the plurality of cycles, associating the amplicon, if present in the sample, with a first capture moiety affixed to a solid support; and d) detecting association of the amplicon, if present in the sample, with the solid support, wherein association of the amplicon with the one or more solid supports indicates the presence of the nucleic acid in the sample. In some embodiments, the first capture moiety comprises a third oligonucleotide sequence, and wherein the second capture moiety comprises a single-stranded oligonucleotide capture sequence that hybridizes with the third oligonucleotide sequence or the complement of the third oligonucleotide sequence in step (c). In some embodiments, detecting association of the amplicon, if present, with the solid support comprises: i) applying a colloidal detection reagent to the solid support, wherein the colloidal detection reagent comprises a first moiety that binds to the label of the amplicon if present and a second moiety that comprises a colloidal metal; and ii) detecting the colloidal detection reagent. In some embodiments, detecting the colloidal detection reagent in step (d)(ii) comprises detection of the colloidal metal. In some embodiments, detecting the colloidal detection reagent in step (d)(ii) comprises: a) applying a developing reagent to the solid support, wherein the developing agent is suitable for forming a precipitate in the presence of the colloidal metal; and b) detecting the colloidal detection reagent by detecting the formation of the precipitate at the solid support. In some embodiments, the formation of the precipitate is detected by visual, electronic, or magnetic detection. In some embodiments, the formation of the precipitate is detected by a mechanical reader. In some embodiments, the developing reagent comprises silver. In some embodiments, the label comprises biotin or a derivative thereof, and wherein the first moiety of the colloidal detection reagent comprises neutravidin, streptavidin, or an antigen-binding domain that specifically binds biotin. In some embodiments, the first moiety of the colloidal detection reagent comprises neutravidin, and wherein the second moiety of the colloidal detection reagent comprises a colloidal gold ion. In some embodiments, the conditions in step (b) are suitable for amplification by polymerase chain reaction (PCR). In some embodiments, the conditions in step (b) are suitable for amplification by recombinase-polymerase assay (RPA), nucleic acid sequenced-based chain assay (NASBA), rolling circle amplification, branched chain amplification, ligation amplification, or loop-mediated isothermal amplification. In some embodiments, the portion of the sample in admixture with the PCR amplification mixture is passed through the continuous capillary tubing using a peristaltic pump, high performance liquid chromatography (HPLC) pump, precision syringe pump, or vacuum. In some embodiments, the method further comprises, prior to step (b): passing the portion of the sample in admixture with the amplification mixture through a preheating zone at between about 20° C. and about 55° C. via the continuous capillary tubing. In some embodiments, the preheating zone is between about 37° C. and about 42° C. In some embodiments, the portion of the sample in admixture with the amplification mixture is passed through the preheating zone for up to 30 minutes. In some embodiments, the portion of the sample in admixture with the amplification mixture is passed through the preheating zone for about 15 minutes. In some embodiments, the method further comprises, prior to step (b): passing the portion of the sample in admixture with the amplification mixture through an activation zone at between about 80° C. and about 100° C. via the continuous capillary tubing. In some embodiments, the activation zone is between about 90° C. and about 95° C. In some embodiments, the portion of the sample in admixture with the amplification mixture is passed through the activation zone for up to 20 minutes. In some embodiments, the portion of the sample in admixture with the amplification mixture is passed through the activation zone for between about 5 minutes and about 10 minutes. In some embodiments, the method further comprises, after step (b) and prior to step (c): passing the portion of the sample in admixture with the amplification mixture through an extension zone at between about 55° C. and about 72° C. via the continuous capillary tubing. In some embodiments, the method further comprises, after step (b) and prior to step (c): i) mixing at least a portion of a second sample with an amplification mixture comprising deoxyribonucleotides, a polymerase, and a second primer pair, wherein the second primer pair comprises a third primer comprising a label and a fourth oligonucleotide sequence that hybridizes with a first strand of a portion of a second nucleic acid, and a fourth primer comprising a fifth oligonucleotide sequence that hybridizes with a second strand of the portion of the second nucleic acid opposite the first strand and a third capture moiety; ii) passing the portion of the second sample in admixture with the amplification mixture through the first, second, and third stationary temperature zones for a second plurality of cycles through the continuous capillary tubing under conditions suitable for amplification of the portion of the second nucleic acid, if present in the sample, wherein each cycle of the second plurality comprises: 1) passing the portion of the second sample in admixture with the amplification mixture through the first stationary temperature zone via the continuous capillary tubing at the first temperature and for the first duration suitable for denaturing the strands of the second nucleic acid, if present in the second sample, 2) after step (ii)(1), passing the portion of the second sample in admixture with the amplification mixture through the second stationary temperature zone via the continuous capillary tubing at the second temperature and for the second duration suitable for annealing the third and fourth primers to the respective strands of the second nucleic acid, if present in the second sample, and 3) after step (ii)(2), passing the portion of the second sample in admixture with the amplification mixture through the third stationary temperature zone via the continuous capillary tubing at the third temperature and for the third duration suitable for amplifying the second nucleic acid, if present in the second sample, via the polymerase and second primer pair; wherein the second nucleic acid, if present in the second sample, is associated concurrently with the amplified first nucleic acid target, if present in the first sample, with a fourth capture moiety that associates with the third capture moiety, wherein the fourth capture moiety is coupled to a solid support; and wherein the association of the amplified second nucleic acid, if present in the second sample, with the solid support is detected concurrently with the hybridization of the amplified first nucleic acid, if present in the first sample, and wherein association of the amplified second nucleic acid target with the solid support indicates the presence of the second nucleic acid target in the second sample. In some embodiments, the first and the second samples are the same. In some embodiments, the first and the second nucleic acids are different. In some embodiments, the method further comprises, after passing the portion of the first sample in admixture with the amplification mixture through the first, second, and third stationary temperature zones for the plurality of cycles, and prior to passing the portion of the second sample in admixture with the amplification mixture through the first, second, and third stationary temperature zones for the second plurality of cycles: passing a volume of air through the continuous capillary tubing sufficient to separate the portion of the first sample in admixture with the amplification mixture and the portion of the second sample in admixture with the amplification mixture. In some embodiments, the method further comprises, after passing the volume of air through the continuous capillary tubing, and prior to passing the portion of the second sample in admixture with the amplification mixture through the first, second, and third stationary temperature zones for the second plurality of cycles: passing a solution comprising sodium hypochlorite at a concentration of between about 0.1% and about 10% through the continuous capillary tubing. In some embodiments, the solution comprises sodium hypochlorite at a concentration of about 1.6%. In some embodiments, the method further comprises, after passing the bleach solution through the continuous capillary tubing, and prior to passing the portion of the second sample in admixture with the amplification mixture through the first, second, and third stationary temperature zones for the second plurality of cycles: passing a solution comprising thiosulfate at a concentration of between about 5 mM and about 500 mM through the continuous capillary tubing. In some embodiments, the solution comprises thiosulfate at a concentration of about 20 mM. In some embodiments, the method further comprises, after passing the thiosulfate solution through the continuous capillary tubing, and prior to passing the portion of the second sample in admixture with the amplification mixture through the first, second, and third stationary temperature zones for the second plurality of cycles: passing water through the continuous capillary tubing. In some embodiments, the method further comprises, after passing the water through the continuous capillary tubing, and prior to passing the portion of the second sample in admixture with the PCR amplification mixture through the first, second, and third stationary temperature zones for the second plurality of cycles, passing a volume of air through the continuous capillary tubing sufficient to separate the water and the portion of the second sample in admixture with the PCR amplification mixture. In some embodiments, step (a) comprises inserting the portion of the sample into the continuous capillary tubing and mixing the portion of the sample with the amplification mixture using a robotic arm or valve system. In some embodiments, the nucleic acid comprises DNA. In some embodiments, the nucleic acid comprises RNA. In some embodiments, the method further comprises, prior to step (a): incubating at least a portion of the sample with a reverse transcriptase, primers, and deoxyribonucleotides under conditions suitable for generation of a cDNA synthesized from the RNA, wherein the cDNA is mixed with the amplification mixture in step (a). In some embodiments, the primers used prior to step (a) are random primers, poly-dT primers, or primers specific for the portion of the RNA. In some embodiments, the portion of the sample is incubated with the reverse transcriptase, primers, and deoxyribonucleotides while being passed through a cDNA synthesis zone between about 37° C. and about 42° C. via the continuous capillary tubing for a time sufficient for generation of a cDNA synthesized from the RNA. In some embodiments, the method further comprises, after passing the portion of the sample in admixture with the reverse transcriptase, primers, and deoxyribonucleotides through the cDNA synthesis zone, and prior to step (b): passing the portion of the sample in admixture with the reverse transcriptase, primers, and deoxyribonucleotides through an activation zone at about 95° C. via the continuous capillary tubing. In some embodiments, during each cycle of the plurality, the portion of the sample in admixture with the amplification mixture is passed through the first stationary temperature zone at between about 80° C. and about 100° C. for 1 second to about 10 minutes. In some embodiments, during each cycle of the plurality, the portion of the sample in admixture with the amplification mixture is passed through the second stationary temperature zone between about 45° C. and about 65° C. for 2 seconds to about 60 seconds. In some embodiments, during each cycle of the plurality, the portion of the sample in admixture with the amplification mixture is passed through the third stationary temperature zone at between about 57° C. and about 74° C. for 3 seconds to about 60 seconds. In some embodiments, during each cycle of the plurality, the portion of the sample in admixture with the PCR amplification mixture is passed through both the second stationary temperature zone and the third stationary temperature zone at between about 45° C. and about 80° C. for between about 0.5 seconds and about 5 minutes. In some embodiments, the plurality of cycles comprises greater than or equal to 2 cycles and less than or equal to 100 cycles. In some embodiments, the method further comprises, prior to step (a), incubating the portion of the sample with a lysis buffer comprising greater than or equal to 0.1% and less than or equal to 10% N,N-dimethyl-N-dodecylglycine betaine (w/v). In some embodiments, the sample is further mixed in step (a) with betaine. In some embodiments, the sample is further mixed in step (a) with a fluorescent or colored dye. In some embodiments, the second primer comprises: the second oligonucleotide sequence, wherein the second oligonucleotide sequence allows for primer extension in the 5′ to 3′ direction; and the third oligonucleotide sequence, wherein the third oligonucleotide sequence is oriented in the opposite 5′ to 3′ direction compared with the direction of primer extension from the second oligonucleotide sequence. In some embodiments, the third oligonucleotide sequence comprises a modified nucleotide at the 3′ terminus that blocks primer extension. In some embodiments, the second primer further comprises one or more linkers between the 5′ end of the third oligonucleotide sequence and the 5′ end of the second oligonucleotide sequence. In some embodiments, the first capture moiety is affixed to a spacer reagent and, wherein the spacer reagent is coupled to the solid support. In some embodiments, the spacer reagent comprises a serum albumin protein. In some embodiments, the spacer reagent comprises a dendrimer. In some embodiments, the sample comprises whole blood, serum, saliva, urine, soil, tissue, or an environmental sample. In some embodiments, the nucleic acid comprises a viral nucleic acid. In some embodiments, the viral nucleic acid is from a virus selected from the group consisting of HIV, HBV, HCV, West Nile, Zika, and parvovirus. In some embodiments, the nucleic acid comprises a bacterial, archaean, protozoan, fungal, plant, or animal nucleic acid.

Certain other aspects of the present disclosure relate to apparatuses for amplifying a nucleic acid from a sample. In some embodiments, the apparatus comprises: capillary tubing arranged around a support in a plurality of circuits, wherein each circuit of the plurality comprises a first, a second, and a third stationary temperature zone, and wherein the capillary tubing is heated to a first temperature in the first stationary temperature zone, a second temperature in the second stationary temperature zone, and a third temperature in the third stationary temperature zone; a robotic arm configured to introduce into the capillary tubing a sample comprising a nucleic acid in admixture with an amplification mixture comprising deoxyribonucleotides, a polymerase, and a primer pair; and a pump or vacuum configured to pass the sample comprising the nucleic acid in admixture with the amplification mixture through the plurality of circuits within the capillary tubing. In some embodiments, the apparatus further comprises one or more processors, a memory, one or more programs, wherein the one or more programs are stored in the memory and configured to be executed by the one or more processors, the one or more programs including instructions for controlling the temperature of the first, second, and third stationary temperature zones. In some embodiments, the apparatus further comprises an incubator for a cDNA synthesis zone in which the capillary tubing is heated to between about 37° C. and about 42° C. upstream of the plurality of circuits. In some embodiments, the apparatus further comprises an incubator for an activation zone in which the capillary tubing is heated to about 95° C. upstream of the plurality of circuits. In some embodiments, the capillary tubing forms a conical, cylindrical, or spiral shape in each circuit of the plurality. In some embodiments, the capillary tubing comprises polytetrafluoroethylene (PTFE). In some embodiments, the plurality of circuits of the capillary tubing comprise from about 25 to about 44 circuits. In some embodiments, the robotic arm comprises a peristaltic or HPLC pump configured to introduce the sample comprising the nucleic acid target in admixture with an amplification mixture into the capillary tubing, and wherein the apparatus further comprises a secondary pump configured to pull the sample comprising the nucleic acid target in admixture with an amplification mixture through the capillary tubing. In some embodiments, the apparatus further comprises an incubator for a PCR extension zone in which the capillary tubing is heated to between about 55° C. and about 72° C. downstream of the plurality of circuits. In some embodiments, the vacuum configured to pass the sample comprising the nucleic acid in admixture with the amplification mixture through the plurality of circuits is a peristaltic pump, high performance liquid chromatography (HPLC) pump, or precision syringe pump.

Certain other aspects of the present disclosure relate to methods for detecting an antigen in a sample. In some embodiments, the method comprises: a) providing a plurality of single-stranded oligonucleotide capture sequences each affixed to a solid support; b) after step (a), contacting the solid supports with an antigen-binding domain that specifically binds an antigen, wherein the antigen-binding domain is coupled to a single-stranded oligonucleotide sequence that hybridizes with at least one of the single-stranded oligonucleotide capture sequences on the solid supports, and wherein the microarray is contacted with the antigen-binding domain under conditions suitable for the single-stranded oligonucleotide sequence of the antigen binding domain to hybridize with the at least one single-stranded oligonucleotide capture sequence on the solid supports; c) after step (a), contacting the solid supports with at least a portion of the sample under conditions suitable for the antigen-binding domain to bind the antigen, if present in the sample; d) after step (a), applying a colloidal detection reagent to the solid supports, wherein the colloidal detection reagent comprises a first moiety that specifically binds to the antigen if present and a second moiety that comprises a colloidal metal; e) after (d), washing the solid supports with a wash solution; and f) after steps (a)-(e), detecting the colloidal detection reagent, wherein detection of the colloidal detection reagent indicates the presence of the antigen in the sample. In some embodiments, the solid supports are arranged as a microarray, a multiplex bead array, or a well array. In some embodiments, the solid supports are nitrocellulose, silica, plastic, or hydrogel. In some embodiments, detecting the colloidal detection reagent in step (f) comprises detection of the colloidal metal. In some embodiments, detecting the colloidal detection reagent in step (f) comprises: 1) applying a developing reagent to the solid supports, wherein the developing agent is suitable for forming a precipitate in the presence of the colloidal metal; and 2) detecting the colloidal detection reagent by detecting the formation of the precipitate. In some embodiments, the formation of the precipitate is detected by visual, electronic, or magnetic detection. In some embodiments, the formation of the precipitate is detected by a mechanical reader. In some embodiments, the developing reagent comprises silver. In some embodiments, the first moiety comprises a second antigen binding domain that specifically binds to the antigen, wherein the second antigen binding domain is coupled to biotin or a derivative thereof, and wherein the colloidal suspension is coupled to avidin, neutravidin, streptavidin, or a derivative thereof bound to the biotin. In some embodiments, the colloidal metal is gold, platinum, palladium, or ruthenium. In some embodiments, the single-stranded oligonucleotide capture sequence at each spot of the plurality is coupled to a spacer reagent, and the spacer reagent is coupled to the solid supports. In some embodiments, the spacer reagent comprises a serum albumin protein. In some embodiments, the spacer reagent comprises a dendrimer. In some embodiments, the method further comprises, prior to step (c), exposing the sample to a lysis buffer comprising greater than or equal to 0.1% and less than or equal to 10% N,N-dimethyl-N-dodecylglycine betaine (w/v). In some embodiments, the lysis buffer comprises greater than or equal to 0.5% and less than or equal to 4% N,N-dimethyl-N-dodecylglycine betaine (w/v). In some embodiments, the lysis buffer comprises greater than or equal to 1% and less than or equal to 2% N,N-dimethyl-N-dodecylglycine betaine (w/v). In some embodiments, the sample is exposed to the lysis buffer at a ratio between 1:50 sample:lysis buffer and 50:1 sample:lysis. In some embodiments, the portion of the sample is exposed to the lysis buffer at a ratio of about 1:1 sample:lysis buffer. In some embodiments, the lysis buffer further comprises 0.1× to 5× phosphate buffered saline (PBS) buffer or Tris EDTA (TE) buffer. In some embodiments, the lysis buffer further comprises 1×PBS. In some embodiments, the solid supports are contacted with the antigen-binding domain in step (b) in the presence of a hybridization buffer comprising 0.1× to 10× saline sodium citrate (SSC) buffer, 0.001% to 30% blocking agent, and 0.01% to 30% crowding agent. In some embodiments, the blocking agent comprises bovine serum albumin (BSA), polyethylene glycol (PEG), casein, or polyvinyl alcohol (PVA). In some embodiments, the blocking agent comprises BSA, and the BSA is present in the buffer at 1% to 3%. In some embodiments, the crowding agent is Polyethylene Glycol Bisphenol A Epichlorohydrin Copolymer. In some embodiments, the Polyethylene Glycol Bisphenol A Epichlorohydrin Copolymer is present in the hybridization buffer at 1% to 3%. In some embodiments, the buffer comprises 2× to 5×SSC buffer. In some embodiments, the method further comprises, prior to steps (b) and (c), blocking the solid supports using a solution comprising BSA. In some embodiments, the solid supports are blocked for 1 hour at 37° C. using 2% BSA solution. In some embodiments, the method further comprises washing the solid supports with a wash solution after blocking the solid supports. In some embodiments, the method further comprises, after steps (b) and (c) and prior to step (d), washing the solid supports with a wash buffer comprising 0.1× to 10×SSC buffer and 0.01% to 30% detergent. In some embodiments, the detergent comprises 0.05% to 5% N-lauroylsarcosine sodium salt. In some embodiments, the wash buffer comprises 1× to 5×SSC buffer. In some embodiments, one or more of the lysis buffer, wash buffer, and hybridization buffer further comprises a control oligonucleotide that hybridizes with at least one of the single-stranded oligonucleotide capture sequences on its solid support. In some embodiments, the antigen is a viral antigen. In some embodiments, the viral antigen is from a virus selected from the group consisting of: HIV, HBV, HCV, West Nile, Zika, and parvovirus. In some embodiments, the antigen is a bacterial, archaean, protozoan, fungal, plant, or animal antigen. In some embodiments, the sample comprises whole blood, serum, saliva, urine, soil, tissue, or an environmental sample.

Further provided herein are kits for detecting an antigen in a sample. In some embodiments, the kit comprises: a) a plurality of single-stranded oligonucleotide capture sequences each affixed to a solid support; and b) a plurality of antigen-binding domains, wherein each antigen-binding domain of the plurality specifically binds an antigen, and wherein each antigen-binding domain of the plurality is coupled to a single-stranded oligonucleotide sequence that is substantially complementary to a single-stranded oligonucleotide sequence affixed to the solid supports. In some embodiments, the kit further comprises: c) a second antigen-binding domain coupled to a colloidal detection reagent, wherein the second antigen-binding domain specifically binds an antigen that is also specifically bound by an antigen-binding domain of the plurality of antigen-binding domains in (b).

Further provided herein is a plurality of single-stranded oligonucleotide capture sequences each affixed to a solid support, wherein each single-stranded oligonucleotide capture sequence is independently selected from the group consisting of SEQ ID NOs:1-15. In some embodiments, the single-stranded oligonucleotide capture sequence at each solid support is coupled to a spacer reagent, and the spacer reagent is coupled to the solid supports. In some embodiments, the spacer reagent comprises a serum albumin protein. In some embodiments, the spacer reagent comprises a dendrimer. Further provided herein is a kit, comprising: a) the plurality of any of the above embodiments; and b) a plurality of antigen binding domains, wherein each antigen binding domain of the plurality is coupled to a single-stranded oligonucleotide sequence independently selected from the group consisting of SEQ ID NOs:16-30. Further provided herein is a kit, comprising: a) the plurality of any of the above embodiments; and b) a plurality of primer pairs, wherein each primer pair of the plurality comprises: 1) a first primer comprising a label and a first oligonucleotide sequence that hybridizes with a first strand of a nucleic acid; and 2) a second primer comprising a second oligonucleotide sequence that hybridizes with a second strand of the portion of the nucleic acid opposite the first strand and a third oligonucleotide sequence, wherein the third oligonucleotide sequence of each first primer is independently selected from the group consisting of SEQ ID NOs:16-30.

Further provided herein is a plurality of single-stranded oligonucleotide capture sequences each affixed to a solid support, wherein each single-stranded oligonucleotide capture sequence is independently selected from the group consisting of SEQ ID NOs:16-30. In some embodiments, the single-stranded oligonucleotide capture sequence at each solid support is coupled to a spacer reagent, and the spacer reagent is coupled to the solid supports. In some embodiments, the spacer reagent comprises a serum albumin protein. In some embodiments, the spacer reagent comprises a dendrimer. Also provided herein is a kit, comprising: a) the plurality of sequences of any of the above embodiments; and b) a plurality of antigen binding domains, wherein each antigen binding domain of the plurality is coupled to a single-stranded oligonucleotide sequence independently selected from the group consisting of SEQ ID NOs:1-15. Further provided herein is a kit, comprising: a) the plurality of sequences of any of the above embodiments; and b) a plurality of primer pairs, wherein each primer pair of the plurality comprises: 1) a first primer comprising a label and a first oligonucleotide sequence that hybridizes with a first strand of a nucleic acid; and 2) a second primer comprising a second oligonucleotide sequence that hybridizes with a second strand of the portion of the nucleic acid opposite the first strand and a third oligonucleotide sequence, wherein the third oligonucleotide sequence of each first primer is independently selected from the group consisting of SEQ ID NOs:1-15.

In some embodiments of any of the pluralities of sequences described above, the solid supports are arranged as a microarray, a multiplex bead array, or a well array. In some embodiments of any of the pluralities of sequences described above, the solid supports are nitrocellulose, silica, plastic, or hydrogel. In some embodiments of any of the kits described above, the solid supports are arranged as a microarray, a multiplex bead array, or a well array. In some embodiments of any of the kits described above, the solid supports are nitrocellulose, silica, plastic, or hydrogel.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a diagram of a universal array for antigen detection, in accordance with some embodiments. BSA: bovine serum albumin.

FIG. 1B shows the detection of a biomarker for Hepatitis B (HBsAg) using a universal array, in accordance with some embodiments.

FIG. 2A shows a diagram of an amplification primer for use in a universal array for nucleic acid testing (NAT), in accordance with some embodiments (tC sequence: SEQ ID NO:34; HIV target sequence: SEQ ID NO:35). FIG. 2B shows a diagram of amplifying a target sequence using the primer shown in FIG. 2A.

FIG. 2C shows a diagram of a universal array for nucleic acid testing (NAT), in accordance with some embodiments.

FIGS. 2D & 2E show the detection of a nucleic acid from Hepatitis C virus (HCV) using a universal array for nucleic acid testing (NAT), in accordance with some embodiments. Readouts obtained using a sample that lacks HCV nucleic acid (FIG. 2D) or a sample that contains an HCV nucleic acid (FIG. 2E) are shown.

FIG. 3A shows 15 individual probes on a universal array for NAT, in accordance with some embodiments.

FIG. 3B shows the effect of different Empigen BB concentrations in sample preparation for use with a universal array for NAT, in accordance with some embodiments.

FIG. 3C shows the effect of different Hybridization Buffer formulations for use with a universal array for NAT, in accordance with some embodiments. The indicated buffer formulations are presented as x/y/z, where x is the strength of SSC buffer (i.e., “3” indicates 3×SSC buffer), y is the percentage of BSA, and z is the percentage of PEG-C.

FIG. 3D shows the effect of different NaOH concentrations on elution efficiency for the enrichment of a nucleic acid of interest from a sample, in accordance with some embodiments.

FIG. 3E shows the effect of different NaOH concentrations in combination with the amount of enriched nucleic acid of interest used in amplification, in accordance with some embodiments.

FIG. 3F shows the effect of different elution strategies in combination with the amount of enriched nucleic acid of interest used in amplification, in accordance with some embodiments.

FIG. 3G shows the effect of different primer concentrations in nucleic acid enrichment protocols, in accordance with some embodiments.

FIG. 4A shows the enrichment of a nucleic acid of interest from a sample in a pipette tip, in accordance with some embodiments.

FIGS. 4B & 4C show the effect of the ratio of biotin-labeled oligonucleotide to neutravidin-labeled colloidal gold, in accordance with some embodiments. Indicated ratios are biotin-labeled probe:neutravidin-labeled colloidal gold. Dashed rectangles indicate experimental results, and solid rectangles indicate BSA-gold controls detected via 2-step detection assay.

FIG. 5A shows a diagram of a continuous amplification system, in accordance with some embodiments.

FIGS. 5B & 5C show exemplary embodiments of continuous amplification systems, in accordance with some embodiments. FIG. 5B shows a robotic arm for obtaining samples, a pump system, optional pre-heating and activation zones, three stationary temperature zones, waste collection unit, temperature zone controller, and power supply. FIG. 5C shows three zones programmable to provide different temperatures, a temperature control module, optional fan control, power supply, pump module, and optional pre-heating and activation zones.

FIG. 6A shows a diagram of asymmetric amplification for NAT, in accordance with some embodiments.

FIG. 6B shows ratios of reverse to forward primers in asymmetric amplification for NAT, in accordance with some embodiments.

### DETAILED DESCRIPTION

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### Microarrays

The universal array platform described herein can be used to detect a variety of nucleic acids or antigens of interest.

Nucleic Acid Detection

Certain aspects of the present disclosure relate to methods for detecting a nucleic acid in a sample. In some embodiments, the methods include: a) amplifying at least a portion of a nucleic acid from a sample using a primer pair under conditions suitable for amplification of an amplicon comprising the portion of the nucleic acid if present in the sample, wherein the primer pair include: 1) a first primer comprising a label and a first oligonucleotide sequence that hybridizes with a first strand of the portion of the nucleic acid, and 2) a second primer comprising a second oligonucleotide sequence that hybridizes with a second strand of the portion of the nucleic acid opposite the first strand and a third oligonucleotide sequence; b) after step (a), contacting the amplicon, if present, to a plurality of single-stranded oligonucleotide capture sequences each affixed to a solid support, and wherein the amplicon, if present, hybridizes with at least one of the single-stranded oligonucleotide capture sequences on its solid support via the third oligonucleotide sequence or the complement of the third oligonucleotide sequence; c) after step (a), applying a colloidal detection reagent to the solid supports, wherein the colloidal detection reagent comprises a first moiety that binds to the label of the amplicon if present and a second moiety that comprises a colloidal metal; d) after (c), washing the solid supports with a wash solution; and e) after steps (a)-(d), detecting the colloidal detection reagent, wherein detection of the colloidal detection reagent on a solid support indicates the presence of the hybridized amplicon, thus detecting the nucleic acid in the sample.

The universal array platform provides an adaptable and versatile approach for, e.g., diagnostic testing. As an example, an oligonucleotide 18 bases long with a known sequence is covalently attached to an activated glass slide or other surface as a small spot. After binding and blocking excess binding sites, a complementary oligonucleotide sequence is covalently attached to an antigen, such as HIV-1 gp41 immunodominant region. If a clinical sample, such as blood, contains antibodies to HIV-1 gp41 and is mixed with the HIV gp41 peptide labeled with the complementary oligo nucleotide, the antibody binds to the gp41 peptide, which when put onto the microarray described above, the complementary oligonucleotide binds to its ligand partner spotted onto the array. The unbound materials are washed off and the array probed with a biotin labeled anti-antibody (i.e., monoclonal anti-human Ig or protein A/G). An anti-biotin molecule (i.e., streptavidin) labeled gold is used to label antibodies bound to gp41, which is bound to a specific spot on the microarray due to the complementary oligonucleotide tethers described above. Excess materials are washed off and the gold labeled microspot is then detected directly or the gold particles used to catalyze silver deposition, which can be easily detected, thereby indicating the presence/absence of anti-HIV antibodies to gp41 in the sample thereby alerting the user whether the person was infected with HIV based upon a detectable amount of anti-HIV-1 gp41 antibodies present in the sample.

Importantly, the same oligonucleotide sequence can be used to label different capture reagents, which all will bind the complementary oligonucleotide sequence on the array. Hence, the next assay could be for detecting HBV, HCV, a toxin, hormone, or nucleic acid, all of which can bind to the same spot. The capture reagent will only bind to the same spot based upon its oligonucleotide label. Hence, a known set of oligonucleotides can be used to create a generic capture microarray and the same set of complementary oligos can be used to label any capture reagents. For example, a 16×16 microarray would have 256 spots, each with a corresponding oligonucleotide sequence. These oligonucleotide sequences can each be unique, or the array may include redundant spots used to confirm the results. Assume that each spot is a duplicate, one then has the potential to differentiate 128 different assays at the same time. That array can then become a standard, generic platform, and used to detect millions of different targets by simply labeling different capture reagents with the 128 different complementary oligonucleotide sequences, which can be provided as a generic kit. Additionally, the same sample can be mixed with different detector solutions that contain different tests and/or overlapping tests. For example, the sample can be screened for infectious diseases my mixing with solution A, then tested for cancer by mixing another portion of the sample with solution B, then testing for toxins by mixing another portion with solution C, and then detecting nucleic acids to any of the targets of interest by mixing with a solution D for direct detection of nucleic acids or after an amplification step. The microarray does not change—it stays fixed—but different detector solutions can be used to test for many different targets. This helps reduce the cost for making the microarrays and greatly expands their utility to detect virtually any target. Exemplary features and aspects of the universal array platform are described in greater detail infra.

As used herein, unless otherwise specified, nucleic acids and/or oligonucleotides refer broadly to polymers of nucleic acids (e.g., DNA or RNA) and are meant to include single-stranded and double-stranded species, as well as species comprising one or more usually naturally occurring and/or modified nucleosides/nucleotides (e.g., locked nucleic acids, peptide nucleic acids or PNAs, etc.).

As used herein, unless otherwise specified, an “amplicon” refers to the product of any of the types of nucleic acid amplification described herein, including but not limited to polymerase chain reaction (PCR), recombinase-polymerase assay (RPA), nucleic acid sequenced-based chain assay (NASBA), rolling circle amplification, branched chain amplification, ligation amplification, and loop-mediated isothermal amplification. In some embodiments, an amplicon is a double-stranded nucleic acid. In some embodiments, an amplicon is a single-stranded nucleic acid.

As used herein, the term “solid support” refers to any solid or semi-solid structure suitable for the attachment of biological molecules thereto, such as nucleic acids. Solid supports need not be flat or a single structure, and may be of any type of shape(s) including spherical shapes (e.g., beads). Solid supports may be arranged in any format. In some embodiments, the solid supports are arranged as a microarray (e.g., flat slide), a multiplex bead array, or a well array. In addition, the solid supports may be made of any suitable material, including, but not limited to, silicon, plastic, glass, polymer, ceramic, photoresist, nitrocellulose, and hydrogel. In some embodiments, the solid supports are nitrocellulose, silica, plastic, or hydrogel.

Colloidal suspensions of nanoparticles such as colloidal gold can be attached to biological probes such as antibodies, useful as detection reagents for rapid and sensitive detection in immunostaining. Methods for preparing and using colloidal detection reagents are well known in the art (see, e.g., Hostetler et al., Langmuir 14:17-30, 1998; Wang et al., Langmuir 17(19):5739-41, 2001). The colloidal detection reagent can be of any material. In some embodiments, the colloidal detection reagent comprises a metal. Examples of colloidal metal include, but are not limited to, gold (Au), silver (Ag), platinum (Pt), palladium (Pd), copper (Cu), nickel (Ni), ruthenium (Ru), and mixtures thereof. In some embodiments, detecting the colloidal detection reagent in step (e) comprises detection (e.g., direct detection) of the colloidal metal. In some embodiments, detecting the colloidal detection reagent in step (e) includes: 1) applying a developing reagent to the solid supports, wherein the developing agent is suitable for forming a precipitate in the presence of the colloidal metal; and 2) detecting the colloidal detection reagent by detecting the formation of the precipitate on a solid support. In some embodiments, the formation of the precipitate is detected by visual, electronic, or magnetic detection. In some embodiments, the formation of the precipitate is detected by a mechanical reader. In some of the embodiments described above, the developing reagent comprises silver. In some of the embodiments described above, silver nitrate and a reducing agent (e.g., hydroquinone) are used. In some of the embodiments described above, a camera (e.g., CCD camera) is used to image the results of colloidal staining.

In some embodiments, the conditions in step (a) are suitable for amplification by polymerase chain reaction (PCR). In some embodiments, the conditions in step (a) are suitable for amplification by recombinase-polymerase assay (RPA), nucleic acid sequenced-based chain assay (NASBA), rolling circle amplification, branched chain amplification, ligation amplification, or loop-mediated isothermal amplification. In some embodiments, the label comprises biotin and the third oligonucleotide sequence hybridizes with at least one of the single-stranded oligonucleotide capture sequences.

In some embodiments, each single-stranded oligonucleotide capture sequence is coupled to a spacer reagent, and the spacer reagent is coupled to the corresponding solid support. In some embodiments, the spacer reagent comprises a serum albumin protein (e.g., BSA). In some embodiments, the spacer reagent comprises a dendrimer. In some embodiments, the method further comprises washing the solid supports with a wash solution after step (b).

In some embodiments, the first primer is a forward primer that amplifies in the sense direction of the nucleic acid, and the second primer is a reverse primer that amplifies in the antisense direction of the nucleic acid. In some embodiments, the second primer is a forward primer that amplifies in the sense direction of the nucleic acid, and the first primer is a reverse primer that amplifies in the antisense direction of the nucleic acid. In some embodiments, the second primer comprises: the second oligonucleotide sequence, wherein the second oligonucleotide sequence allows for primer extension in the 5′ to 3′ direction; and the third oligonucleotide sequence, wherein the third oligonucleotide sequence is oriented in the opposite 5′ to 3′ direction compared with the direction of primer extension from the second oligonucleotide sequence. In some embodiments, the third oligonucleotide sequence comprises a modified nucleotide at the 3′ terminus that blocks primer extension. In some embodiments, the second primer further comprises one or more linkers between the 5′ end of the third oligonucleotide sequence and the 5′ end of the second oligonucleotide sequence.

In some embodiments, the label of the first primer comprises biotin. In some embodiments, the first moiety of the colloidal detection reagent comprises neutravidin or derivatives thereof, streptavidin or derivatives thereof, avidin or derivatives thereof, or an antigen-binding domain (e.g., antibody or antibody fragment) that specifically binds biotin. In some embodiments, the first moiety of the colloidal detection reagent comprises neutravidin, and wherein the second moiety of the colloidal detection reagent comprises a colloidal gold ion. In some embodiments, the colloidal detection reagent is applied to the solid supports in step (c) at a final dilution of 0.00001OD to 20OD. In some embodiments, the first moiety of the colloidal detection reagent comprises neutravidin, wherein the second moiety of the colloidal detection reagent comprises a colloidal gold ion, and wherein the colloidal detection reagent is applied to the solid supports in step (c) at a final dilution of 0.05OD to 0.2OD. Varying amounts of the colloidal detection reagent can be used. In some embodiments, 1 pL to 1000 μL of colloidal detection reagent is applied to the solid supports in step (c) per μL of amplicon. In some embodiments, 100 μL of colloidal detection reagent is applied to the solid supports in step (c) per 1.5 μL of amplicon.

In some embodiments, the method further comprises, prior to step (a), exposing the sample to a lysis buffer. In some embodiments, the lysis buffer comprises N,N-dimethyl-N-dodecylglycine betaine. In some embodiments, the lysis buffer comprises greater than or equal to 0.1% and less than or equal to 10% N,N-dimethyl-N-dodecylglycine betaine (w/v). In some embodiments, the lysis buffer comprises greater than or equal to 0.5% and less than or equal to 4% N,N-dimethyl-N-dodecylglycine betaine (w/v). In some embodiments, the lysis buffer comprises greater than or equal to 1% and less than or equal to 2% N,N-dimethyl-N-dodecylglycine betaine (w/v).

In some embodiments, the sample is exposed to the lysis buffer at a ratio between 1:50 sample:lysis buffer and 50:1 sample:lysis. In some embodiments, the portion of the sample is exposed to the lysis buffer at a ratio of about 1:1 sample:lysis buffer. In some embodiments, the lysis buffer further comprises 0.1× to 5× phosphate buffered saline (PBS) buffer or Tris EDTA (TE) buffer. In some embodiments, the lysis buffer further comprises 1×PBS.

In some embodiments, an amplicon is hybridized to the solid supports in the presence of a hybridization buffer. A variety of hybridization buffers are known in the art. In some embodiments, the amplicon is hybridized to the solid supports in step (b) in a hybridization buffer comprising 0.1× to 10× saline sodium citrate (SSC) buffer, 0.001% to 30% blocking agent, and 0.01% to 30% crowding agent. In some embodiments, the blocking agent comprises bovine serum albumin (BSA), polyethylene glycol (PEG), casein, or polyvinyl alcohol (PVA). In some embodiments, the blocking agent comprises BSA, and the BSA is present in the hybridization buffer at 1% to 3%. In some embodiments, the crowding agent is Polyethylene Glycol Bisphenol A Epichlorohydrin Copolymer. In some embodiments, the Polyethylene Glycol Bisphenol A Epichlorohydrin Copolymer is present in the hybridization buffer at 1% to 3%. In some embodiments, the hybridization buffer comprises 2× to 5×SSC buffer.

In some embodiments, the method further comprises, prior to step (b), blocking the solid supports. In some embodiments, the solid supports are blocked using a solution comprising BSA. In some embodiments, the solid supports are blocked for 1 hour at 37° C. using 2% BSA solution. In some embodiments, the method further comprises washing the solid supports with a wash solution after blocking the solid supports.

In some embodiments, the method further comprises, after step (b) and prior to step (c), washing the solid supports with a wash buffer. In some embodiments, the wash buffer comprises 0.1× to 10×SSC buffer and 0.01% to 30% detergent. In some embodiments, the detergent comprises 0.05% to 5% N-lauroylsarcosine sodium salt. In some embodiments, the wash buffer comprises 1× to 5×SSC buffer.

In some embodiments, the method further comprises, prior to step (a): (i) contacting the sample with an oligonucleotide coupled to a solid substrate, wherein the oligonucleotide hybridizes with the nucleic acid if present in the sample; (ii) washing the solid substrate under conditions suitable to remove non-specific interactions with the solid substrate but retain the nucleic acid hybridized with the oligonucleotide, if present in the sample; and (iii) eluting the nucleic acid, if present in the sample, from the oligonucleotide, wherein the eluted nucleic acid is subjected to PCR amplification in step (a). In some embodiments, the method further comprises, prior to step (a): (i) contacting the sample with an oligonucleotide, wherein the oligonucleotide hybridizes with the nucleic acid if present in the sample, (ii) simultaneous with or after step (i), contacting the sample with a solid substrate, wherein the solid substrate is coupled to a first binding moiety, wherein the oligonucleotide is coupled to a second binding moiety that binds the first binding moiety, and wherein the sample is contacted with the solid substrate under conditions suitable for the second binding moiety to bind the first binding moiety; (iii) washing the solid substrate under conditions suitable to remove non-specific interactions with the solid substrate but retain the oligonucleotide and the nucleic acid hybridized with the oligonucleotide, if present in the sample; and (iv) eluting the nucleic acid, if present in the sample, from the oligonucleotide, wherein the eluted nucleic acid is subjected to PCR amplification in step (a). In some embodiments, the oligonucleotide is coupled to the solid substrate via a covalent interaction. In some embodiments, the oligonucleotide is coupled to the solid substrate via an avidin:biotin or streptavidin:biotin interaction, or wherein the first binding moiety comprises avidin, neutravidin, streptavidin, or a derivative thereof and the second binding moiety comprises biotin or a derivative thereof. In some embodiments, the solid substrate is positioned in a pipet tip, and wherein step (i) comprises pipetting the sample into the pipet tip. In some embodiments, the solid substrate comprises a matrix or plurality of beads. In some embodiments, the nucleic acid comprises DNA. In some embodiments, the nucleic acid comprises RNA.

In some embodiments, the method further comprises, prior to step (a), incubating at least a portion of the sample with a reverse transcriptase, primers, and deoxyribonucleotides under conditions suitable for generation of a cDNA synthesized from the nucleic acid, wherein the portion of the nucleic acid is amplified in step (a) using the cDNA. In some embodiments, the primers used prior to step (a) are random primers, poly-dT primers, or primers specific for the portion of the nucleic acid. In some embodiments, the portion of the sample is incubated with the reverse transcriptase, primers, and the deoxyribonucleotides in the presence of an RNase inhibitor. In some embodiments, the portion of the sample is incubated with the reverse transcriptase, primers, and the deoxyribonucleotides in the presence of betaine. In some embodiments, the betaine is present at a concentration of about 0.2M to about 1.5M. In some embodiments, the nucleic acid comprises a viral nucleic acid. In some embodiments, the viral nucleic acid is from a virus selected from the group consisting of HIV, HBV, HCV, West Nile, Zika, and parvovirus. In some embodiments, the nucleic acid comprises a bacterial, archaean, protozoan, fungal, plant, or animal nucleic acid. In some embodiments, the sample comprises whole blood, serum, saliva, urine, soil, tissue, or an environmental sample (e.g., comprising water, soil, etc.).

In some embodiments, any of the reagents described herein (e.g., a lysis or hybridization buffer) can comprise a positive control oligonucleotide, e.g., that hybridizes with a capture oligonucleotide of the array. Advantageously, this can be used as a positive control to ensure that the correct reagents were used. For example, a plurality of capture oligonucleotides can be used to represent different reagents (e.g., lysis buffer, hybridization buffer, and the like), and a specific positive control in each reagent can be used to “encode” that the proper reagent is used. When viewed in aggregate, the plurality can indicate that some or all of the steps of microarray preparation/analysis were completed with reagents spiked with positive control oligonucleotide(s), thereby indicating the use of correct reagents.

In some embodiments, the present disclosure contemplates adjusting the ratio of the first primer relative to the second primer used in the step of amplifying a nucleic acid in a sample. The ratio of the primers can be skewed to preferentially amplify one strand of the nucleic acid more than the other—a technique known as asymmetric amplification (see, e.g., McCabe, PCR Protocols: A guide to Methods and Applications, 76-83, 1990). Advantageously, the skewed primer ratio in asymmetric amplification results in a predominantly uniform product, which may help increase the strength of the hybridization signal detected on a universal array of the present disclosure. In some embodiments of the present disclosure, the portion of the nucleic acid is amplified using an excess of the first primer relative to the second primer, and wherein the amplicon, if present, is a single-stranded nucleic acid that hybridizes with at least one of the single-stranded oligonucleotide capture sequences via the complement of the third oligonucleotide sequence. In some embodiments, the portion of the nucleic acid is amplified using a ratio of the first primer to the second primer of between about 12.5:1 and about 100:1. In some embodiments, a ratio of the first primer to the second primer of at least about 2:1, at least about 5:1, at least about 10:1, at least about 15:1, at least about 20:1, at least about 25:1, at least about 30:1, at least about 35:1, at least about 40:1, at least about 45:1, at least about 50:1, at least about 55:1, at least about 60:1, at least about 65:1, at least about 70:1, at least about 75:1, at least about 80:1, at least about 85:1, at least about 90:1, at least about 95:1, at least about 100:1, at least about 150:1, or at least about 200:1 is used.

Antigen Detection

In addition to nucleic acids, the universal array platform described herein can be used to detect a variety of antigens of interest. Accordingly, some aspects of the present disclosure relate to methods for detecting an antigen in a sample. In some embodiments, the method includes: a) providing a plurality of single-stranded oligonucleotide capture sequences each affixed to a solid support; b) after step (a), contacting the solid supports with an antigen-binding domain that specifically binds an antigen, wherein the antigen-binding domain is coupled to a single-stranded oligonucleotide sequence that hybridizes with at least one of the single-stranded oligonucleotide capture sequences on the solid supports, and wherein the microarray is contacted with the antigen-binding domain under conditions suitable for the single-stranded oligonucleotide sequence of the antigen binding domain to hybridize with the at least one single-stranded oligonucleotide capture sequence on the solid supports; c) after step (a), contacting the solid supports with at least a portion of the sample under conditions suitable for the antigen-binding domain to bind the antigen, if present in the sample; d) after step (a), applying a colloidal detection reagent to the solid supports, wherein the colloidal detection reagent comprises a first moiety that specifically binds to the antigen if present and a second moiety that comprises a colloidal metal; e) after (d), washing the solid supports with a wash solution; and f) after steps (a)-(e), detecting the colloidal detection reagent, wherein detection of the colloidal detection reagent indicates the presence of the antigen in the sample.

In some embodiments, the antigen comprises a polypeptide, lipid, or carbohydrate. In certain embodiments, the antigen is a polypeptide antigen.

Any of the compositions and methods described above in reference to nucleic acid detection may also be used for antigen detection, including, without limitation, the solid supports, the colloidal detection reagent and method of detecting thereof, the developing reagent, the spacer reagent, the lysis buffer, the blocking agent, the crowding agent, the hybridization buffer, and the wash buffer.

In some embodiments, the first moiety comprises a second antigen-binding domain that specifically binds to the antigen, wherein the second antigen-binding domain is coupled to biotin or a derivative thereof, and wherein the colloidal suspension is coupled to avidin, neutravidin, streptavidin, or a derivative thereof bound to the biotin. In some embodiments, the colloidal metal is gold, platinum, palladium, or ruthenium. In some embodiments, the single-stranded oligonucleotide capture sequence at each spot of the plurality is coupled to a spacer reagent, and the spacer reagent is coupled to the solid supports. In some embodiments, the spacer reagent comprises a serum albumin protein. In some embodiments, the spacer reagent comprises a dendrimer. In some embodiments, the method further comprises, prior to step (c), exposing the sample to a lysis buffer comprising greater than or equal to 0.1% and less than or equal to 10% N,N-dimethyl-N-dodecylglycine betaine (w/v). In some embodiments, the antigen is a viral antigen. In some embodiments, the viral antigen is from a virus selected from the group consisting of: HIV, HBV, HCV, West Nile, Zika, and parvovirus. In some embodiments, the antigen is a bacterial, archaean, protozoan, fungal, plant, or animal antigen. In some embodiments, the sample comprises whole blood, serum, saliva, urine, soil, tissue, or an environmental sample (e.g., comprising water, soil, etc.).

### Devices

Other aspects of the present disclosure relate to a device or apparatus for amplifying a nucleic acid in a sample. In some embodiments, the device or apparatus includes: capillary tubing arranged around a support in a plurality of circuits, wherein each circuit of the plurality comprises a first, a second, and a third stationary temperature zone, and wherein the capillary tubing is heated to a first temperature in the first stationary temperature zone, a second temperature in the second stationary temperature zone, and a third temperature in the third stationary temperature zone; a robotic arm configured to introduce into the capillary tubing a sample comprising a nucleic acid in admixture with an amplification mixture comprising deoxyribonucleotides, a polymerase, and a primer pair; and a pump or vacuum configured to pass the sample comprising the nucleic acid in admixture with the amplification mixture through the plurality of circuits within the capillary tubing.

To detect a nucleic acid in a sample using methods described herein, amplification of at least a portion of the nucleic acid can be used for hybridization to the array. Thus, in some aspects, the present disclosure contemplates apparatuses that may be useful for amplifying nucleic acids of interest. In some embodiments, the present disclosure relates to an apparatus for nucleic acid amplification having capillary tubing, a robotic arm, and a pump or vacuum.

Amplification of the nucleic acid is carried out in the capillary tubing. The capillary tubing is arranged around a support in a plurality of circuits, wherein each circuit of the plurality comprises a first, a second, and a third stationary temperature zone, and wherein the capillary tubing is heated to a first temperature in the first stationary temperature zone, a second temperature in the second stationary temperature zone, and a third temperature in the third stationary temperature zone. Each of these zones may correspond to one portion of a standard PCR or other amplification reaction, namely denaturing, annealing, and extension for PCR. For isothermal amplification, each zone can be heated to the same temperature (e.g., 37° C.). Advantageously, capillary tubing allows for an improved rate of heat conduction, which means target reaction temperatures can be rapidly achieved, and reaction times can be shortened. The capillary tubing in each circuit of the plurality may form any shape, for example, without limitation, a conical shape, a cylindrical shape, or a spiral shape. The capillary tubing may comprise any material, for example and without limitation, polytetrafluoroethylene (PTFE). The plurality of circuits of the capillary tubing may comprise any number of circuits, for example and without limitation, from about 25 to about 44 circuits (e.g., corresponding to the number of amplification cycles).

The pump or vacuum of the apparatus can be configured to pass the sample comprising the nucleic acid in admixture with the amplification mixture through the plurality of circuits within the capillary tubing. A variety of pumps and vacuums well known in the art can be used for the apparatus. In some embodiments, the pump or vacuum is a peristaltic pump. In some embodiments, the pump or vacuum is a high performance liquid chromatography (HPLC) pump. In some embodiments, the pump or vacuum is a precision syringe pump.

The robotic arm of the apparatus can be configured to introduce into the capillary tubing a sample comprising a nucleic acid in admixture with an amplification mixture comprising deoxyribonucleotides, a polymerase, and a primer pair. In some embodiments, the robotic arm comprises a peristaltic or HPLC pump configured to introduce the sample comprising the nucleic acid target in admixture with an amplification mixture into the capillary tubing, and wherein the apparatus further comprises a secondary pump configured to pull the sample comprising the nucleic acid target in admixture with an amplification mixture through the capillary tubing.

Additional components may be added to the apparatus. In some embodiments, the apparatus further contains one or more processors, a memory, one or more programs, wherein the one or more programs are stored in the memory and configured to be executed by the one or more processors, the one or more programs including instructions for controlling the temperature of the first, second, and third stationary temperature zones. In some embodiments, the apparatus further contains an incubator for a cDNA synthesis zone (e.g., if the sample is RNA) in which the capillary tubing is heated to between about 37° C. and about 42° C. upstream of the plurality of circuits. In some embodiments, the incubator is a temperature bath, a Peltier device, or a resistance heater. In some embodiments, the sample in admixture with an amplification mixture is held in the cDNA synthesis zone for 15 seconds to 30 minutes. In some embodiments, the apparatus further contains an incubator for an activation zone in which the capillary tubing is heated to about 95° C. upstream of the plurality of circuits. In some embodiments, the apparatus further contains an incubator for a PCR extension zone in which the capillary tubing is heated to between about 55° C. and about 72° C. downstream of the plurality of circuits.

The pump, robotic arm, and various temperature zones may all be controlled by a system control panel, which allows alteration of each of the components, for example, the temperature of the different zones can be changed. In addition, the control panel can be used to change the pumping speed, thereby altering the length of time spent in each temperature zone of the main amplification area. Thus, in some embodiments, the apparatus may further include one or more processors, a memory, one or more programs, wherein the one or more programs are stored in the memory and configured to be executed by the one or more processors, the one or more programs including instructions for controlling the temperature of the first, second, and third stationary temperature zones.

The temperature of the capillary tubing can be maintained at desired temperature in the different temperature zones according to standard techniques known in the art. In some embodiments, the temperature of one of more temperature zones is maintained by Peltier or resistance heaters. In some embodiments, polyimide tape (e.g., inside copper tubing) is used to insulate one or more temperature zones.

In some embodiments, a robotic arm is used to apply an amplicon to the microarray (e.g., solid supports of the present disclosure) after completion of the circuits. In some embodiments, a dye is added, e.g., to aid in visualizing the liquid spotted onto the microarray. In some embodiments, the amplicon is collected into a container with hybridization buffer after completion of the circuits, then added to the microarray (e.g., manually by pipetting, or with a robotic arm).

The apparatuses described above may be used in any of the methods of the present disclosure. For example, in some embodiments, the method includes: a) incubating at least a portion of the sample with an amplification mixture comprising deoxyribonucleotides, a polymerase, and a primer pair, wherein the primer pair comprises a first primer comprising a label and a first oligonucleotide sequence that hybridizes with a first strand of a portion of the nucleic acid, and a second primer comprising a second oligonucleotide sequence that hybridizes with a second strand of the portion of the nucleic acid opposite the first strand and a first capture moiety; b) passing the portion of the sample in admixture with the amplification mixture through first, second, and third stationary temperature zones for a plurality of cycles through continuous capillary tubing under conditions suitable for amplification of an amplicon comprising the portion of the nucleic acid, if present in the sample, wherein each cycle of the plurality includes: 1) passing the portion of the sample in admixture with the amplification mixture through the first stationary temperature zone via the continuous capillary tubing at a first temperature and for a first duration suitable for denaturing the strands of the nucleic acid, if present in the sample, 2) after step (b)(1), passing the portion of the sample in admixture with the amplification mixture through the second stationary temperature zone via the continuous capillary tubing at a second temperature and for a second duration suitable for annealing the first and second primers to the respective strands of the nucleic acid, if present in the sample, and 3) after step (b)(2), passing the portion of the sample in admixture with the amplification mixture through the third stationary temperature zone via the continuous capillary tubing at a third temperature and for a third duration suitable for amplifying the nucleic acid target, if present in the sample, via the polymerase and primer pair; c) after the plurality of cycles, associating the amplicon, if present in the sample, with a first capture moiety affixed to a solid support; and d) detecting association of the amplicon, if present in the sample, with the solid support, wherein association of the amplicon with the one or more solid supports indicates the presence of the nucleic acid in the sample.

General steps for using the above-described apparatuses to amplify and detect a nucleic acid in a sample are described below and may employ any of the reagents or techniques described supra.

The methods can include incubating in an initial container at least a portion of the sample with an amplification mixture comprising deoxyribonucleotides, a polymerase, and a primer pair, wherein the primer pair comprises a first primer comprising a label and a first oligonucleotide sequence that hybridizes with a first strand of a portion of the nucleic acid, and a second primer comprising a second oligonucleotide sequence that hybridizes with a second strand of the portion of the nucleic acid opposite the first strand and a first capture moiety.

Then, a pump and a robotic arm are used to pass the portion of the sample in admixture with the amplification mixture through first, second, and third stationary temperature zones for a plurality of cycles through continuous capillary tubing under conditions suitable for amplification of an amplicon comprising the portion of the nucleic acid, if present in the sample, wherein each cycle of the plurality comprises: 1) passing the portion of the sample in admixture with the amplification mixture through the first stationary temperature zone via the continuous capillary tubing at a first temperature and for a first duration suitable for denaturing the strands of the nucleic acid, if present in the sample, 2) after step (b)(1), passing the portion of the sample in admixture with the amplification mixture through the second stationary temperature zone via the continuous capillary tubing at a second temperature and for a second duration suitable for annealing the first and second primers to the respective strands of the nucleic acid, if present in the sample, and 3) after step (b)(2), passing the portion of the sample in admixture with the amplification mixture through the third stationary temperature zone via the continuous capillary tubing at a third temperature and for a third duration suitable for amplifying the nucleic acid target, if present in the sample, via the polymerase and primer pair.

Next, after the plurality of cycles, the amplicon, if present in the sample, can be associated with a first capture moiety affixed to a solid support.

Finally, association of the amplicon, if present in the sample, with the solid support can be detected, wherein association of the amplicon with the one or more solid supports indicates the presence of the nucleic acid in the sample.

In some embodiments, the first capture moiety comprises a third oligonucleotide sequence, and wherein the second capture moiety comprises a single-stranded oligonucleotide capture sequence that hybridizes with the third oligonucleotide sequence or the complement of the third oligonucleotide sequence in step (c). In some embodiments, detecting association of the amplicon, if present, with the solid support comprises: i) applying a colloidal detection reagent to the solid support, wherein the colloidal detection reagent comprises a first moiety that binds to the label of the amplicon if present and a second moiety that comprises a colloidal metal; and ii) detecting the colloidal detection reagent. In some embodiments, detecting the colloidal detection reagent in step (d)(ii) comprises detection of the colloidal metal. In some embodiments, detecting the colloidal detection reagent in step (d)(ii) comprises: a) applying a developing reagent to the solid support, wherein the developing agent is suitable for forming a precipitate in the presence of the colloidal metal; and b) detecting the colloidal detection reagent by detecting the formation of the precipitate at the solid support. In some embodiments, the formation of the precipitate is detected by visual, electronic, or magnetic detection. In some embodiments, the formation of the precipitate is detected by a mechanical reader. In some embodiments, the developing reagent comprises silver. In some embodiments, the label comprises biotin or a derivative thereof, and wherein the first moiety of the colloidal detection reagent comprises neutravidin, streptavidin, or an antigen-binding domain that specifically binds biotin. In some embodiments, the first moiety of the colloidal detection reagent comprises neutravidin, and wherein the second moiety of the colloidal detection reagent comprises a colloidal gold ion. In some embodiments, the conditions in step (b) are suitable for amplification by polymerase chain reaction (PCR). In some embodiments, the conditions in step (b) are suitable for amplification by recombinase-polymerase assay (RPA), nucleic acid sequenced-based chain assay (NASBA), rolling circle amplification, branched chain amplification, ligation amplification, or loop-mediated isothermal amplification. In some embodiments, the portion of the sample in admixture with the PCR amplification mixture is passed through the continuous capillary tubing using a peristaltic pump, high performance liquid chromatography (HPLC) pump, precision syringe pump, or vacuum. In some of the embodiments described above, the method may further contain, prior to step (b): passing the portion of the sample in admixture with the amplification mixture through a preheating zone at between about 20° C. and about 55° C. via the continuous capillary tubing. In some embodiments, the preheating zone is between about 37° C. and about 42° C. In some embodiments, the portion of the sample in admixture with the amplification mixture is passed through the preheating zone for up to 30 minutes. In some embodiments, the portion of the sample in admixture with the amplification mixture is passed through the preheating zone for about 15 minutes. In some of the embodiments described above, the method may further contain, prior to step (b): passing the portion of the sample in admixture with the amplification mixture through an activation zone at between about 80° C. and about 100° C. via the continuous capillary tubing. In some embodiments, the activation zone is between about 90° C. and about 95° C. In some embodiments, the portion of the sample in admixture with the amplification mixture is passed through the activation zone for up to 20 minutes. In some embodiments, the portion of the sample in admixture with the amplification mixture is passed through the activation zone for between about 5 minutes and about 10 minutes. In some of the embodiments described above, the method may further contain, after step (b) and prior to step (c): passing the portion of the sample in admixture with the amplification mixture through an extension zone at between about 55° C. and about 72° C. via the continuous capillary tubing. In some of the embodiments described above, the method may further contain, after step (b) and prior to step (c): i) mixing at least a portion of a second sample with an amplification mixture comprising deoxyribonucleotides, a polymerase, and a second primer pair, wherein the second primer pair comprises a third primer comprising a label and a fourth oligonucleotide sequence that hybridizes with a first strand of a portion of a second nucleic acid, and a fourth primer comprising a fifth oligonucleotide sequence that hybridizes with a second strand of the portion of the second nucleic acid opposite the first strand and a third capture moiety; ii) passing the portion of the second sample in admixture with the amplification mixture through the first, second, and third stationary temperature zones for a second plurality of cycles through the continuous capillary tubing under conditions suitable for amplification of the portion of the second nucleic acid, if present in the sample, wherein each cycle of the second plurality comprises: 1) passing the portion of the second sample in admixture with the amplification mixture through the first stationary temperature zone via the continuous capillary tubing at the first temperature and for the first duration suitable for denaturing the strands of the second nucleic acid, if present in the second sample, 2) after step (ii)(1), passing the portion of the second sample in admixture with the amplification mixture through the second stationary temperature zone via the continuous capillary tubing at the second temperature and for the second duration suitable for annealing the third and fourth primers to the respective strands of the second nucleic acid, if present in the second sample, and 3) after step (ii)(2), passing the portion of the second sample in admixture with the amplification mixture through the third stationary temperature zone via the continuous capillary tubing at the third temperature and for the third duration suitable for amplifying the second nucleic acid, if present in the second sample, via the polymerase and second primer pair; wherein the second nucleic acid, if present in the second sample, is associated concurrently with the amplified first nucleic acid target, if present in the first sample, with a fourth capture moiety that associates with the third capture moiety, wherein the fourth capture moiety is coupled to a solid support; and wherein the association of the amplified second nucleic acid, if present in the second sample, with the solid support is detected concurrently with the hybridization of the amplified first nucleic acid, if present in the first sample, and wherein association of the amplified second nucleic acid target with the solid support indicates the presence of the second nucleic acid target in the second sample.

The first and the second samples may or may not be the same. In some embodiments, the first and the second samples are the same. In some embodiments, the first and the second samples are different samples. The first and the second nucleic acids may or may not be the same. In some embodiments, the first and the second nucleic acids are the same. In some embodiments, the first and the second nucleic acids are different. It is to be noted that one may detect the same nucleic acid from different samples using the apparatuses and methods described herein.

In some of the embodiments described above, the method may further contain, after passing the portion of the first sample in admixture with the amplification mixture through the first, second, and third stationary temperature zones for the plurality of cycles, and prior to passing the portion of the second sample in admixture with the amplification mixture through the first, second, and third stationary temperature zones for the second plurality of cycles: passing a volume of air through the continuous capillary tubing sufficient to separate the portion of the first sample in admixture with the amplification mixture and the portion of the second sample in admixture with the amplification mixture. In some embodiments, the method may further contain, after passing the volume of air through the continuous capillary tubing, and prior to passing the portion of the second sample in admixture with the amplification mixture through the first, second, and third stationary temperature zones for the second plurality of cycles: passing a solution comprising sodium hypochlorite at a concentration of between about 0.1% and about 10% through the continuous capillary tubing. In some embodiments, the solution comprises sodium hypochlorite at a concentration of about 1.6%. In some embodiments, the method may further contain, after passing the bleach solution through the continuous capillary tubing, and prior to passing the portion of the second sample in admixture with the amplification mixture through the first, second, and third stationary temperature zones for the second plurality of cycles: passing a solution comprising thiosulfate at a concentration of between about 5 mM and about 500 mM through the continuous capillary tubing. In some embodiments, the solution comprises thiosulfate at a concentration of about 20 mM. In some embodiments, the method may further include, after passing the thiosulfate solution through the continuous capillary tubing, and prior to passing the portion of the second sample in admixture with the amplification mixture through the first, second, and third stationary temperature zones for the second plurality of cycles: passing water through the continuous capillary tubing. In some embodiments, the method may further contain, after passing the water through the continuous capillary tubing, and prior to passing the portion of the second sample in admixture with the PCR amplification mixture through the first, second, and third stationary temperature zones for the second plurality of cycles: passing a volume of air through the continuous capillary tubing sufficient to separate the water and the portion of the second sample in admixture with the PCR amplification mixture. In some embodiments, a water and air passing scheme comprises four water pulses of 20 seconds each, separated by 20 seconds of air pulses, between samples. In some embodiments that may be combined with any of the preceding embodiments, step (a) comprises inserting the portion of the sample into the continuous capillary tubing and mixing the portion of the sample with the amplification mixture using a robotic arm or valve system.

In some embodiments that may be combined with any of the preceding embodiments, the nucleic acid comprises DNA. In some embodiments that may be combined with any of the preceding embodiments, the nucleic acid comprises RNA. In some embodiments, the method may further contain, prior to step (a): incubating at least a portion of the sample with a reverse transcriptase, primers, and deoxyribonucleotides under conditions suitable for generation of a cDNA synthesized from the RNA, wherein the cDNA is mixed with the amplification mixture in step (a). In some embodiments, the primers used prior to step (a) are random primers, poly-dT primers, or primers specific for the portion of the RNA. In some embodiments, wherein the portion of the sample is incubated with the reverse transcriptase, primers, and deoxyribonucleotides while being passed through a cDNA synthesis zone between about 37° C. and about 42° C. via the continuous capillary tubing for a time sufficient for generation of a cDNA synthesized from the RNA. In some embodiments, the method may further contain, after passing the portion of the sample in admixture with the reverse transcriptase, primers, and deoxyribonucleotides through the cDNA synthesis zone, and prior to step (b): passing the portion of the sample in admixture with the reverse transcriptase, primers, and deoxyribonucleotides through an activation zone at about 95° C. via the continuous capillary tubing. In some embodiments that may be combined with any of the preceding embodiments, during each cycle of the plurality, the portion of the sample in admixture with the amplification mixture is passed through the first stationary temperature zone at between about 80° C. and about 100° C. for 1 second to about 10 minutes. In some embodiments, the portion of the sample in admixture with the amplification mixture is passed through the first stationary temperature zone at between about 90° C. and about 97° C. for 2 seconds to about 20 seconds. In some embodiments, the portion of the sample in admixture with the amplification mixture is passed through the first stationary temperature zone at between about 95° C. for at least 10 seconds. In some embodiments that may be combined with any of the preceding embodiments, during each cycle of the plurality, the portion of the sample in admixture with the amplification mixture is passed through the second stationary temperature zone between about 45° C. and about 65° C. for 2 seconds to about 60 seconds. In some embodiments, the portion of the sample in admixture with the amplification mixture is passed through the second stationary temperature zone between about 55° C. for at least 15 seconds. In some embodiments, the portion of the sample in admixture with the amplification mixture is passed through the second stationary temperature zone between about 50° C. and about 57° C. for 2 seconds to about 60 seconds. In some embodiments that may be combined with any of the preceding embodiments, during each cycle of the plurality, the portion of the sample in admixture with the amplification mixture is passed through the third stationary temperature zone at between about 57° C. and about 74° C. for 3 seconds to about 60 seconds. In some embodiments, the portion of the sample in admixture with the amplification mixture is passed through the third stationary temperature zone at between about 65° C. and about 72° C. for 3 seconds to about 60 seconds. In some embodiments, the portion of the sample in admixture with the amplification mixture is passed through the third stationary temperature zone at between about 65° C. and about 72° C. for at least 15 seconds. In some embodiments, if isothermal or LAMP amplification is used, all three stationary temperature zones could have the same temperature, e.g., 37° C. In addition, for all stationary temperature zones, the speed of the pump or vacuum can be controlled to alter the length of time the sample in admixture with the amplification mixture spent in each stationary temperature zone.

In some embodiments that may be combined with any of the preceding embodiments, during each cycle of the plurality, the portion of the sample in admixture with the PCR amplification mixture is passed through both the second stationary temperature zone and the third stationary temperature zone at between about 45° C. and about 80° C. for between about 0.5 seconds and about 5 minutes. In some embodiments that may be combined with any of the preceding embodiments, the plurality of cycles comprises greater than or equal to 2 cycles and less than or equal to 100 cycles. In some embodiments that may be combined with any of the preceding embodiments, the method may further contain, prior to step (a), incubating the portion of the sample with a lysis buffer comprising greater than or equal to 0.1% and less than or equal to 10% N,N-dimethyl-N-dodecylglycine betaine (w/v). In some embodiments that may be combined with any of the preceding embodiments, the sample is further mixed in step (a) with betaine. In some embodiments that may be combined with any of the preceding embodiments, the sample is further mixed in step (a) with a fluorescent or colored dye. In some embodiments that may be combined with any of the preceding embodiments, the second primer comprises: the second oligonucleotide sequence, wherein the second oligonucleotide sequence allows for primer extension in the 5′ to 3′ direction; and the third oligonucleotide sequence, wherein the third oligonucleotide sequence is oriented in the opposite 5′ to 3′ direction compared with the direction of primer extension from the second oligonucleotide sequence. In some embodiments, the third oligonucleotide sequence comprises a modified nucleotide at the 3′ terminus that blocks primer extension. In some embodiments, the second primer further comprises one or more linkers between the 5′ end of the third oligonucleotide sequence and the 5′ end of the second oligonucleotide sequence. In some embodiments that may be combined with any of the preceding embodiments, the first capture moiety is affixed to a spacer reagent and, wherein the spacer reagent is coupled to the solid support. In some embodiments, the spacer reagent comprises a serum albumin protein. In some embodiments, the spacer reagent comprises a dendrimer. In some embodiments that may be combined with any of the preceding embodiments, the sample comprises whole blood, serum, saliva, urine, soil, tissue, or an environmental sample. In some embodiments that may be combined with any of the preceding embodiments, the nucleic acid comprises a viral nucleic acid. In some embodiments, the viral nucleic acid is from a virus selected from the group consisting of HIV, HBV, HCV, West Nile, Zika, and parvovirus. In some embodiments that may be combined with any of the preceding embodiments, the nucleic acid comprises a bacterial, archaean, protozoan, fungal, plant, or animal nucleic acid. In some embodiments, the sample comprises whole blood, serum, saliva, urine, soil, tissue, or an environmental sample (e.g., comprising water, soil, etc.).

### Kits and Articles of Manufacture

Other aspects of the present disclosure relate to kits or articles of manufacture for detecting a nucleic acid or antigen in a sample.

In some embodiments, the present disclosure relates to a kit having: a plurality of primer pairs, wherein each primer pair of the plurality comprises a first primer coupled to a label, wherein the first primer hybridizes with a first strand of a nucleic acid, and a second primer comprising: 1) a first oligonucleotide sequence that allows for primer extension in the 5′ to 3′ direction and hybridizes with a second strand of the nucleic acid opposite the first strand; 2) a second oligonucleotide sequence, wherein the second oligonucleotide sequence is oriented in the opposite 5′ to 3′ direction compared with the direction of primer extension from the second oligonucleotide sequence; and 3) one or more linkers between the 5′ end of the first oligonucleotide sequence and the 5′ end of the second oligonucleotide sequence. In some embodiments, the second oligonucleotide sequence comprises a modified nucleotide at the 3′ terminus that blocks primer extension. In some embodiments, the label coupled to the first primer comprises biotin. In some of the embodiments described above, the kit may further include a plurality of single-stranded oligonucleotide capture sequences each affixed to a solid support, and wherein at least one single-stranded oligonucleotide sequence on its solid support hybridizes with the second oligonucleotide sequence of a second primer of a primer pair of the plurality.

In some embodiments, the present disclosure relates to a kit having: a) a plurality of single-stranded oligonucleotide capture sequences each affixed to a solid support; and b) a plurality of primer pairs, wherein each primer pair of the plurality comprises: 1) a first primer comprising a label and a first oligonucleotide sequence that hybridizes with a first strand of the portion of the nucleic acid, and 2) a second primer comprising a second oligonucleotide sequence that hybridizes with a second strand of the portion of the nucleic acid opposite the first strand and a third oligonucleotide sequence, wherein the third oligonucleotide sequence of each primer pair of the plurality hybridizes with a single-stranded oligonucleotide capture sequence on its solid support. In some embodiments, the second oligonucleotide sequence of each primer pair of the plurality allows for primer extension in the 5′ to 3′ direction, wherein the third oligonucleotide sequence of each primer pair of the plurality is oriented in the opposite 5′ to 3′ direction compared with the direction of primer extension from the second oligonucleotide sequence, and wherein the second primer of each primer pair of the plurality further comprises one or more linkers between the 5′ end of the third oligonucleotide sequence and the 5′ end of the second oligonucleotide sequence. In some embodiments, the third oligonucleotide sequence of each primer pair of the plurality comprises a modified nucleotide at the 3′ terminus that blocks primer extension. In some of the embodiments described above, each of the single-stranded oligonucleotide capture sequences on its support is coupled to a spacer reagent, and the spacer reagent is coupled to the solid support. In some embodiments, the spacer reagent comprises a serum albumin protein. In some embodiments, the spacer reagent contains a dendrimer.

In some embodiments, the present disclosure relates to a kit having: a) a plurality of single-stranded oligonucleotide capture sequences each affixed to a solid support; and b) a plurality of antigen-binding domains, wherein each antigen-binding domain of the plurality specifically binds an antigen, and wherein each antigen-binding domain of the plurality is coupled to a single-stranded oligonucleotide sequence that is substantially complementary to a single-stranded oligonucleotide sequence affixed to the solid supports. In some embodiments, the kit further contains c) a second antigen-binding domain coupled to a colloidal detection reagent, wherein the second antigen-binding domain specifically binds an antigen that is also specifically bound by an antigen-binding domain of the plurality of antigen-binding domains in (b).

In some embodiments, a kit of the present disclosure further includes primer sequences. For example, for detection of HBV, a kit of the present disclosure can further include amplification primers for detecting an HBV nucleic acid, e.g., the HBV sAg. In some embodiments, an amplicon comprising the sequence TTC CTA GGA CCC CTG CTC GTG TTA CAG GCG GGG TTT TTC TTG TTG ACA AGA ATC CTC ACA ATA CCG CAG AGT CTA GAC TCG TGG TGG ACT TCT CTC AAT TTT CTA GGG GG (SEQ ID NO:33) is amplified. In some embodiments, the amplification primers comprise a first primer comprising the sequence CCC CCT AGA AAA TTG AGA GAA GTC CAC CAC G (SEQ ID NO:32) and a second primer comprising the sequence ATT CCT AGG ACC CCT GCT CGT GTT A (SEQ ID NO:31). In some embodiments, the first primer comprises biotin coupled to the 5′ end.

Oligonucleotides

Other aspects of the present disclosure relate to single-stranded oligonucleotides, e.g., tether or capture sequences. These sequences can be used interchangeably as tether or capture sequences. For example, provided herein are pluralities of single-stranded oligonucleotide capture sequences, where each sequence of the plurality is independently selected from SEQ ID NOs:1-15. Also provided herein are pluralities of single-stranded oligonucleotide capture sequences, where each sequence of the plurality is independently selected from SEQ ID NOs:16-30. Advantageously, these sequences have been identified from among thousands of potential sequences for robust and consistent hybridization, lack of secondary structure, and the absence of homology with naturally-occurring sequences, e.g., related to the human genome.

Further provided herein is a plurality of single-stranded oligonucleotide capture sequences each affixed to a solid support, wherein each single-stranded oligonucleotide capture sequence is independently selected from the group consisting of SEQ ID NOs:1-15. In some embodiments, the single-stranded oligonucleotide capture sequence at each solid support is coupled to a spacer reagent, and the spacer reagent is coupled to the solid supports. In some embodiments, the spacer reagent comprises a serum albumin protein. In some embodiments, the spacer reagent comprises a dendrimer. Further provided herein is a kit having: a) the plurality of any of the above embodiments; and b) a plurality of antigen binding domains, wherein each antigen binding domain of the plurality is coupled to a single-stranded oligonucleotide sequence independently selected from the group consisting of SEQ ID NOs:16-30. Further provided herein is a kit having: a) the plurality of any of the above embodiments; and b) a plurality of primer pairs, wherein each primer pair of the plurality comprises: 1) a first primer comprising a label and a first oligonucleotide sequence that hybridizes with a first strand of a nucleic acid; and 2) a second primer comprising a second oligonucleotide sequence that hybridizes with a second strand of the portion of the nucleic acid opposite the first strand and a third oligonucleotide sequence, wherein the third oligonucleotide sequence of each first primer is independently selected from the group consisting of SEQ ID NOs:16-30.

Further provided herein is a plurality of single-stranded oligonucleotide capture sequences each affixed to a solid support, wherein each single-stranded oligonucleotide capture sequence is independently selected from the group consisting of SEQ ID NOs:16-30. In some embodiments, the single-stranded oligonucleotide capture sequence at each solid support is coupled to a spacer reagent, and the spacer reagent is coupled to the solid supports. In some embodiments, the spacer reagent comprises a serum albumin protein. In some embodiments, the spacer reagent contains a dendrimer. Further provided herein is a kit having: the plurality of sequences of any of the above embodiments; and b) a plurality of antigen binding domains, wherein each antigen binding domain of the plurality is coupled to a single-stranded oligonucleotide sequence independently selected from the group consisting of SEQ ID NOs:1-15. Further provided herein is a kit having: a) the plurality of sequences of any of the above embodiments; and b) a plurality of primer pairs, wherein each primer pair of the plurality comprises: 1) a first primer comprising a label and a first oligonucleotide sequence that hybridizes with a first strand of a nucleic acid; and 2) a second primer comprising a second oligonucleotide sequence that hybridizes with a second strand of the portion of the nucleic acid opposite the first strand and a third oligonucleotide sequence, wherein the third oligonucleotide sequence of each first primer is independently selected from the group consisting of SEQ ID NOs:1-15.

In some embodiments that may be combined with any of the preceding embodiments, the solid supports are arranged as a microarray, a multiplex bead array, or a well array. In some embodiments that may be combined with any of the preceding embodiments, the solid supports are nitrocellulose, silica, plastic, or hydrogel. In some embodiments that may be combined with any of the preceding embodiments, the solid supports are arranged as a microarray, a multiplex bead array, or a well array. In some embodiments that may be combined with any of the preceding embodiments, the solid supports are nitrocellulose, silica, plastic, or hydrogel.

The present disclosure will be more fully understood by reference to the following Examples. They should not, however, be construed as limiting any aspect or scope of the present disclosure in any way.

### EXAMPLES

The present disclosure will be more fully understood by reference to the following examples. The examples should not, however, be construed as limiting the scope of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: A “Universal Array” Technology for Detection of Protein and Nucleic Acid Biomarkers

The universal array concept employs an array with probe DNA oligonucleotide sequences printed to the array surface. Target DNA oligonucleotides complementary to the probe oligos hybridize with the printed probes. Also attached to the target DNA oligonucleotide sequences are reagents that allow the detection of specific macromolecules. For example, an antibody that specifically binds a disease- or cancer-related biomarker can be conjugated to a DNA oligonucleotide sequence complementary to one of the sequences printed to the array to allow for specific detection of the biomarker. Since a portion of the target DNA oligos hybridize to the probes, different target DNA oligos can be employed for different assays, thereby allowing the same array (e.g., a “universal” array) to be configured for a variety of different assays to detect of biomarkers, proteins, antibodies, and/or nucleic acids of interest.

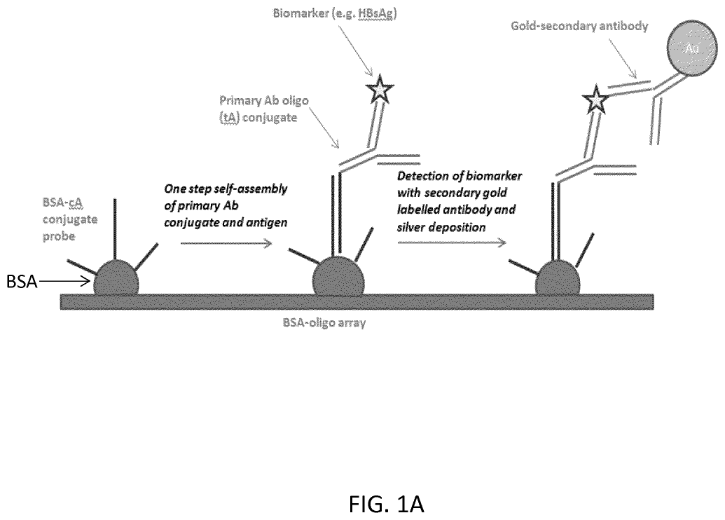

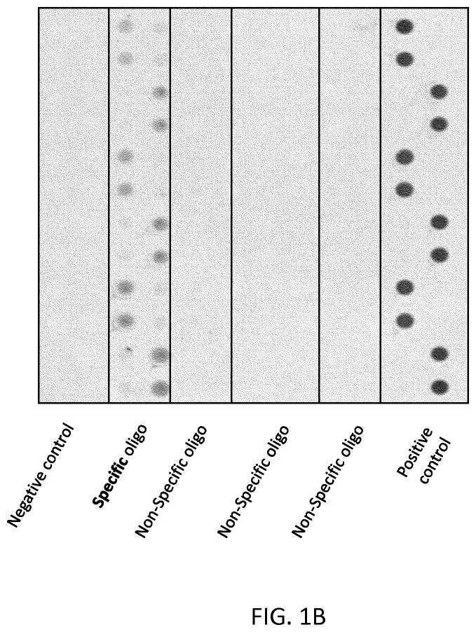

This concept is illustrated in FIG. 1A. FIG. 1A depicts a single-stranded oligonucleotide probe (“cA”) conjugated to BSA, producing a “BSA-cA conjugate probe” which is then printed to the array. For detection of a biomarker (HBsAg protein in this example), a primary antibody that specifically binds HBsAg was conjugated to an oligonucleotide sequence complementary to the probe sequence (“tA conjugate”), allowing the to conjugate to hybridize to the probe at the array location on which the BSA-cA conjugate probe is printed. Various types of labels can be used to detect the presence of the biomarker at the array location. In this example, a gold-labeled secondary antibody that binds the biomarker was used to detect the presence of the biomarker at the array location after precipitation of silver onto the gold, which can be visualized as a spot via colorimetric detection with a CCD camera (see, e.g., Alexandre, I. et al. (2001) Anal. Biochem. 295:1-8).

FIG. 1B shows the results of an exemplary assay. As shown in FIG. 1B, HBsAg protein was only detected at spots on the array when a specific oligo that binds that probe at the spots was used.

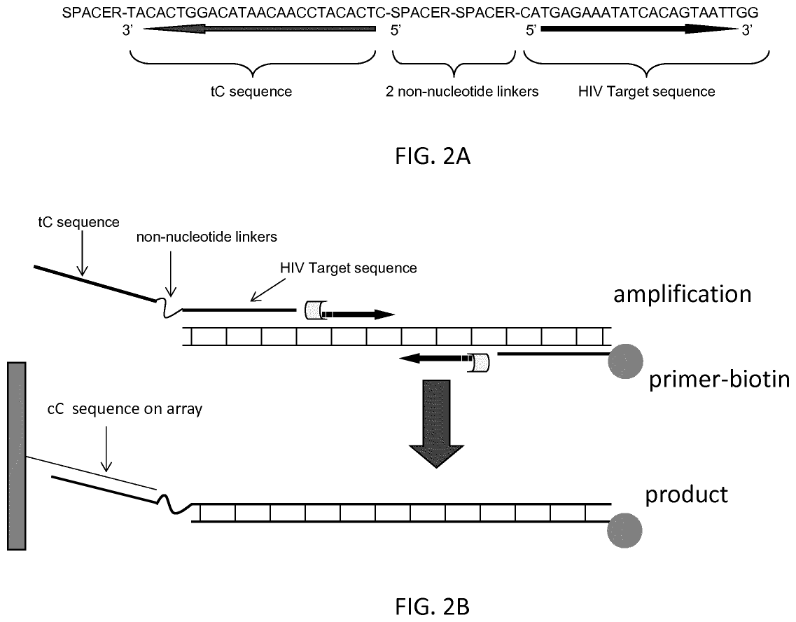

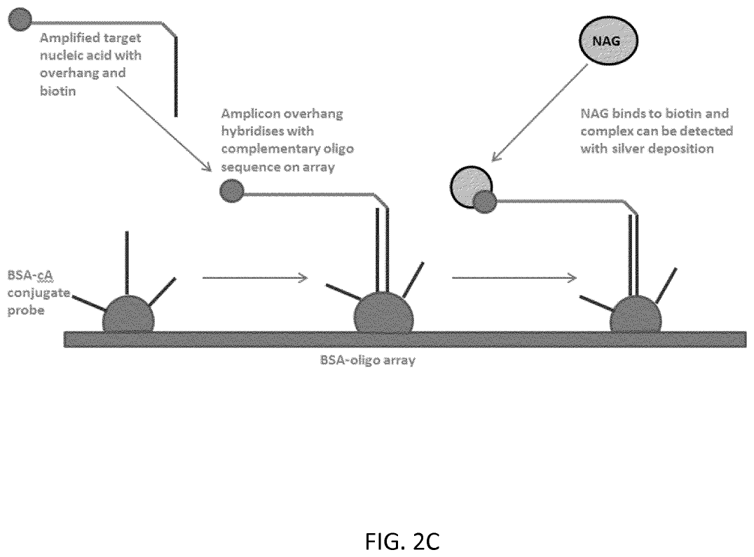

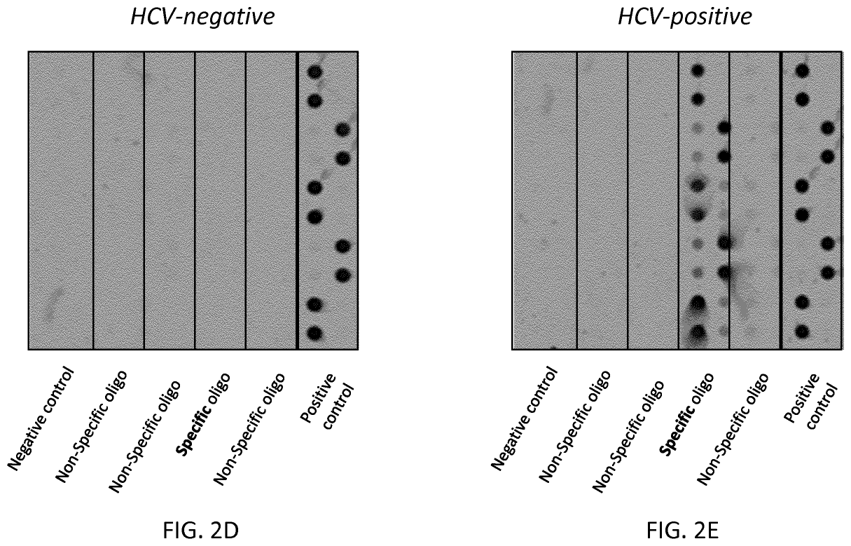

Similarly, the universal array concept can also be applied to nucleic acid testing (NAT), as shown in FIGS. 2A-2E. In this example, a nucleic acid of interest is amplified using polymerase chain reaction (PCR) with an amplification primer that has 3 components: a sequence complementary to a probe on the universal array (“tC sequence”) that is oriented in the 3′ to 5′ direction, a spacer (e.g., 2 non-nucleotide linkers) that prevents PCR extension into the tC sequence, and an oligonucleotide primer specific to the nucleic acid of interest oriented in the 5′ to 3′ direction (FIG. 2A). Thus, upon amplification using the primer, the target sequence is incorporated into the amplicon product, allowing hybridization to a probe printed on the array (FIG. 2B). The PCR reaction also includes another primer specific for the nucleic acid of interest with a detectable label (in this case, biotin) to allow detection of the amplicon hybridized to the array.

A diagram of the universal array is shown in FIG. 2C. The amplicon contains the portion of the target nucleic acid amplified by the two primers, leading to amplicons with a biotin label at one end and an overhang (the tC sequence) that hybridizes to the array at the other end. Upon hybridization to the printed probe, the amplicon can be detected by a variety of means. In this example, gold-conjugated NeutrAvidin (“NAG”) is detected using silver deposition, e.g., as described above.

Representative readouts of this assay are shown in FIGS. 2D & 2E. In this example, no hybridization was detected using an oligo specific for HCV in a sample that lacks HCV nucleic acid (“HCV-negative;” FIG. 2D). However, in a sample that contains HCV nucleic acid, hybridization was only detected when a specific oligo that hybridizes to certain spots on the array was used; non-specific oligos yielded no signal (FIG. 2E).

The adaptation of the universal array concept to protein biomarker detection and NAT is described in greater detail in the Examples provided below.

### Example 2: Probe DNA Oligonucleotide Sequences

The universal array concept described in Example 1 was tested by making arrays containing 15 different probe DNA oligonucleotide sequences (“15 element arrays”).

### Methods

To produce the arrays, a set of probe DNA oligonucleotide sequences is conjugated to a carrier protein and arrayed on a slide. Then, a set of target DNA oligonucleotides complementary to the probe oligos is used to amplify a sample (e.g., in the form of an amplification primer with three components), which allows the target DNA to hybridize with the printed probes.

The probe sequences and complementary sequences used in the 15 element arrays are provided in Table 1.

TABLE 1
Probe sequences and complementary sequences used in the 15 element
arrays.

| | Probe sequence | Complementary sequence |
|---|---|---|
| NS1 | GTTAAGAGCGTCTTCCTTGTTTA | CTAAACAAGGAAGACGCTCTTAA |
| | G (SEQ ID NO: 1) | C (SEQ ID NO: 16) |
| NS2 | GTGACGACTTAAATGTGGAGTA | GATACTCCACATTTAAGTCGTCAC |
| | TC (SEQ ID NO: 2) | (SEQ ID NO: 17) |
| NS3 | ATGGTCATTACGACGAGGATAA | GCTTATCCTCGTCGTAATGACCAT |
| | GC (SEQ ID NO: 3) | (SEQ ID NO: 18) |
| NS4 | GTCAATGACTAGTTTCGAGTATA | CTATACTCGAAACTAGTCATTGAC |
| | G (SEQ ID NO: 4) | (SEQ ID NO: 19) |
| NS5 | GCGGCAACGAGCAAATATGCGT | ATACGCATATTTGCTCGTTGCCGC |
| | AT (SEQ ID NO: 5) | (SEQ ID NO: 20) |
| NS6 | GGGACGATCCAAGACTCATCAG | CTCTGATGAGTCTTGGATCGTCCC |
| | AG (SEQ ID NO: 6) | (SEQ ID NO: 21) |
| NS7 | TTGTTCATCGAGGTAAGGTCAGG | GCCTGACCTTACCTCGATGAACAA |
| | C (SEQ ID NO: 7) | (SEQ ID NO: 22) |
| NS8 | CGCAGCTAAGATCGGAGAGACG | TACGTCTCTCCGATCTTAGCTGCG |
| | TA (SEQ ID NO: 8) | (SEQ ID NO: 23) |
| NS9 | AACCCGAGATCGTAGTATACTC | TTGAGTATACTACGATCTCGGGTT |
| | AA (SEQ ID NO: 9) | (SEQ ID NO: 24) |
| NS10 | TCACCAAAGCTCGGTCACTTGTT | CAACAAGTGACCGAGCTTTGGTG |
| | G (SEQ ID NO: 10) | A (SEQ ID NO: 25) |
| NS11 | GCGATCGCGTTTAGTTGTATTTC | AGAAATACAACTAAACGCGATCG |
| | T (SEQ ID NO: 11) | C (SEQ ID NO: 26) |
| NS12 | TTTATCGAATAAGTCTAATGCTC | AGAGCATTAGACTTATTCGATAA |
| | T (SEQ ID NO: 12) | A (SEQ ID NO: 27) |
| NS13 | TACCTCTATCCCCAACGTGCAAC | TGTTGCACGTTGGGGATAGAGGT |
| | A (SEQ ID NO: 13) | A (SEQ ID NO: 28) |
| NS14 | ATGTCCATCCGTTTTGCCATATG | TCATATGGCAAAACGGATGGACA |
| | A (SEQ ID NO: 14) | T (SEQ ID NO: 29) |
| NS15 | CTATAGCCTCCGTGGATAAACTG | CCAGTTTATCCACGGAGGCTATAG |
| | G (SEQ ID NO: 15) | (SEQ ID NO: 30) |

The 15 element arrays were tested using HBV DNA (5 ug in 1:1 plasma lysate). HBV primers comprised the sequences ATT CCT AGG ACC CCT GCT CGT GTT A (SEQ ID NO:31; forward) and CCC CCT AGA AAA TTG AGA GAA GTC CAC CAC G (SEQ ID NO:32; reverse). The probe sequences were synthesized and conjugated to HBV forward primer. The complementary sequence was the oligonucleotide conjugated to the array. Biotin was conjugated to 5′ end of the reverse primer, and primers were used at 200 nm concentration. Target amplicons generated using Promega GoTaq® 1-step RT-qPCR system (one-step reverse transcription-qPCR reagent system) per Manufacturer Instructions.

### Results

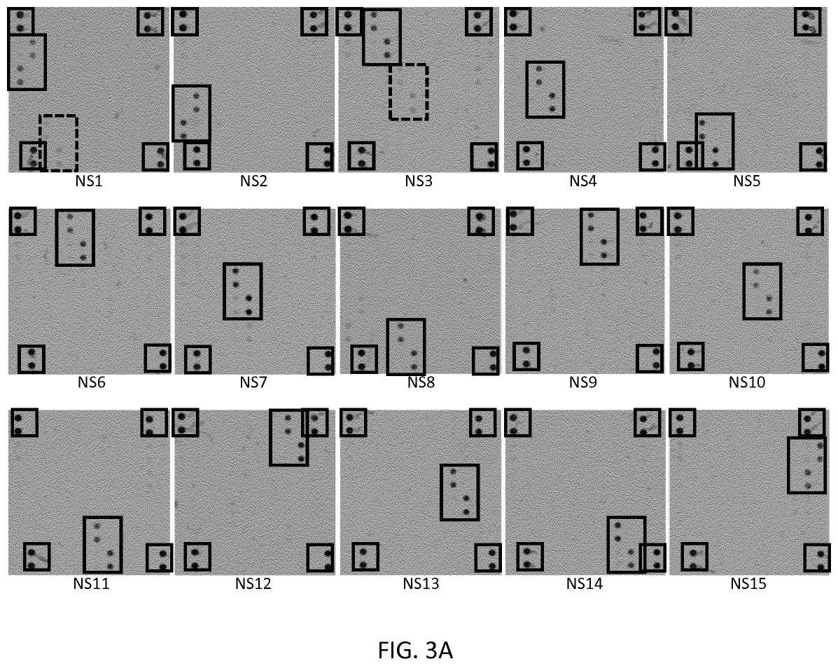

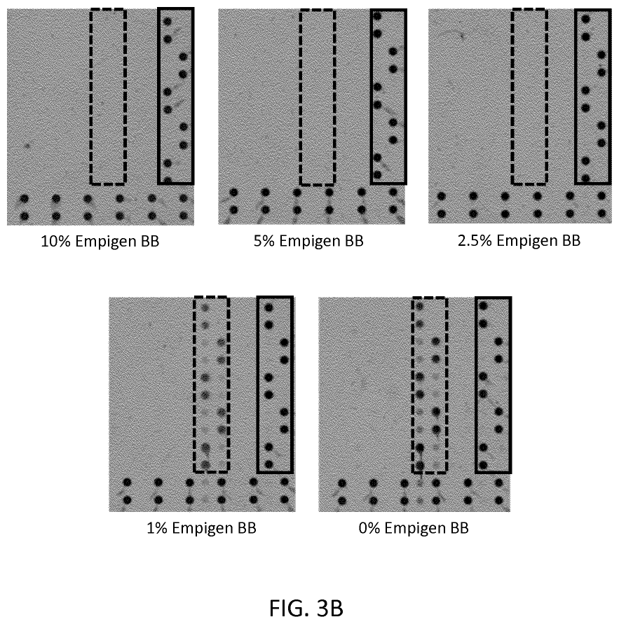

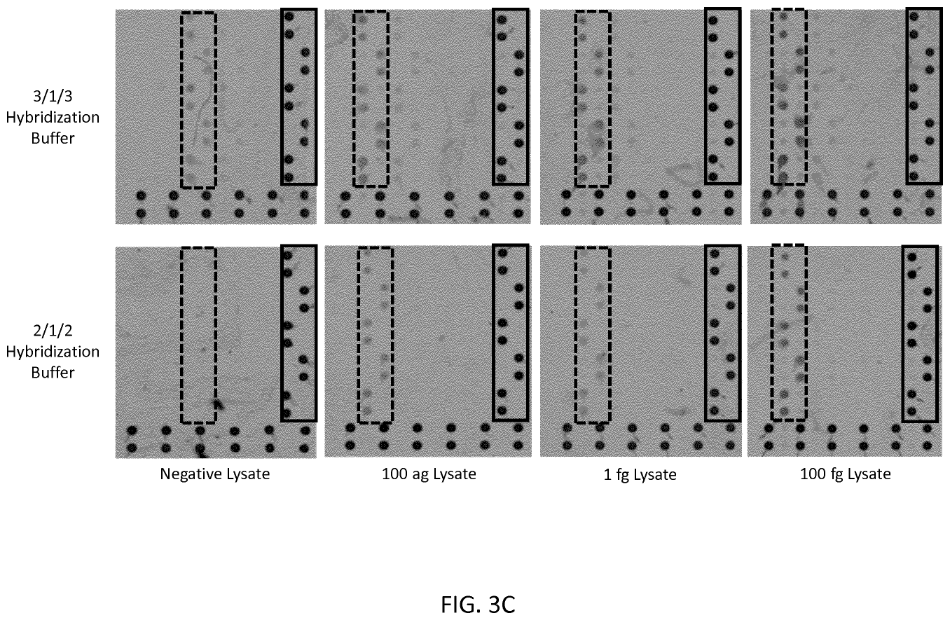

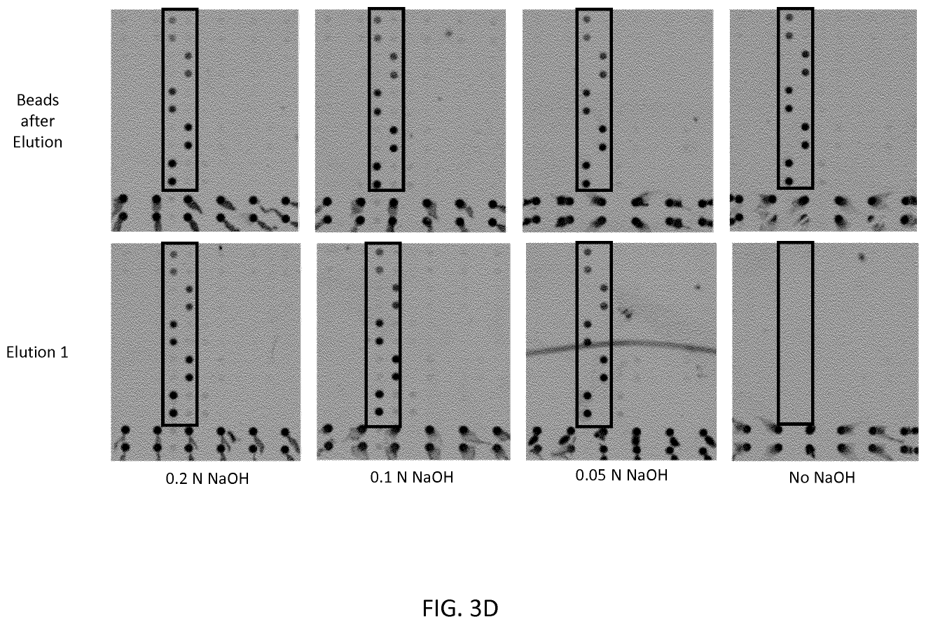

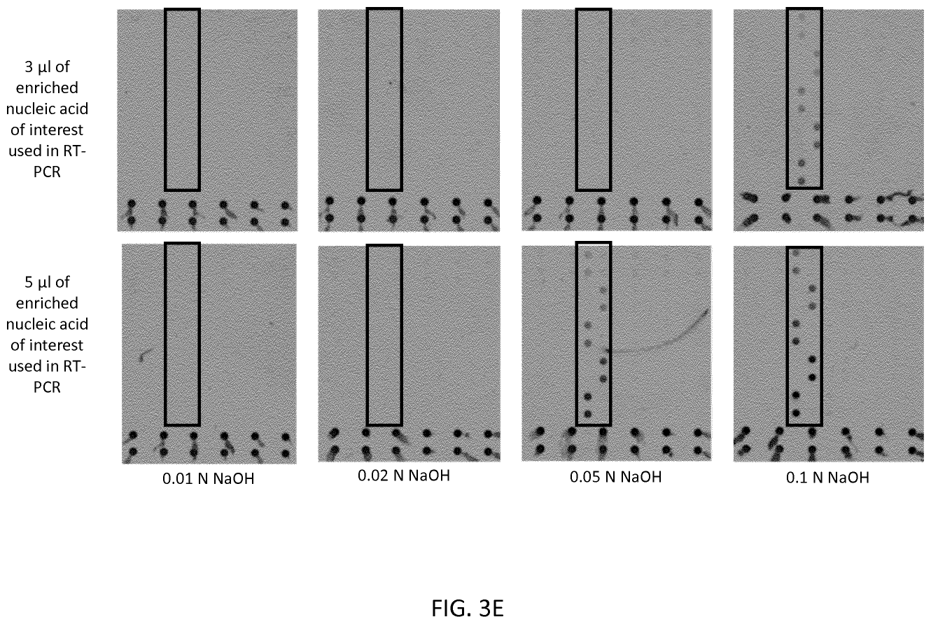

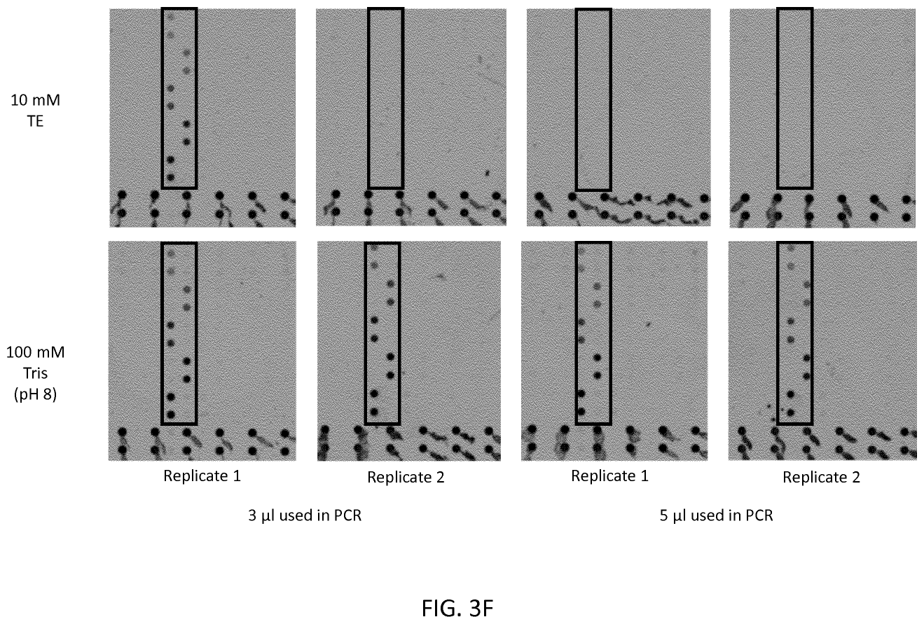

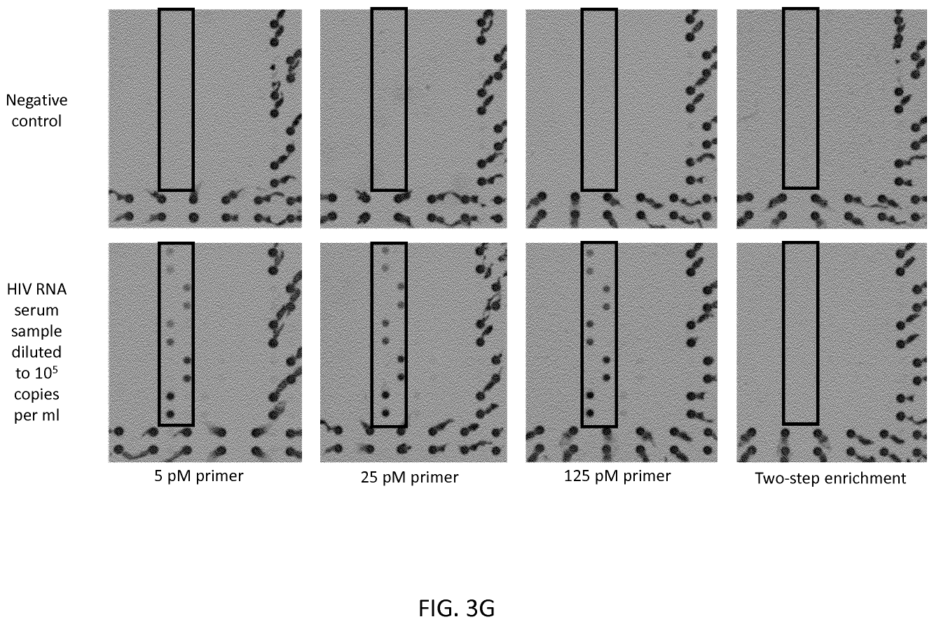

The results are shown in FIG. 3A. Each panel represents a different complementary sequence used on the same 15 element array. The dark spots seen in all four corners of each panel were positive controls of BSA-gold conjugates (denoted by corner boxes in FIG. 3A), which were visualized as dark spots after precipitation of silver (see Example 1). Briefly, each complementary sequence specifically detected the correct probe sequence on the 15 element arrays (denoted by non-corner boxes in FIG. 3A). In some cases cross-reactivity was seen in the complementary sequences, such as NS1 to NS5 or NS3 to NS7 (denoted by dotted boxes in FIG. 3A).

This test using a 15 element array demonstrates the probe component of the universal array concept. Here, 15 probe sequences were used as well as 15 complementary sequences conjugated to HBV primers (i.e., one target sequence), but each complementary sequence could be conjugated to a different target primer. The universal probes can therefore be used to effectively differentiate a range of target sequences. Thus, the use of universal probes removes the need to design new array probes for each experiment/target.

### Example 3: Universal Array Reagents

The universal array concept described in Example 1 uses specific reagents to prepare samples and hybridize samples to the array. In this example, specific reagent concentrations were tested.

Methods

The first part of sample preparation used Lysis Buffer in a 1:1 ratio of sample to buffer. The Lysis Buffer (Table 3) included Phosphate Buffered Saline (Table 2).

TABLE 2
10X Phosphate Buffered Saline.

| Ingredient | Amount | |
|---|---|---|
| Phosphate Buffer | 100 | mM |
| Potassium Chloride | 27 | mM |

| | |
|---|---|
| Sodium Chloride | 1.37M |

Adjust to a final pH of 7.4.

TABLE 3
Lysis Buffer.

| Ingredient | Amount |
|---|---|
| 10X Phosphate Buffered Saline | 1X |
| Empigen BB | 0.5% to 1% |

The second part of sample preparation used PCR to amplify the sample. This was done using either the three component primer system described in Example 1 or the asymmetric amplification system described in Example 10. When using the three component primer system, 100 nM to 200 nM of primer was used. When using the asymmetric amplification system, 40 nM to 80 nM of the forward primer, and 1 μM to 8 μM of the reverse primer were used. If the beginning sample was RNA, a reverse transcriptase mix was used at 0.1× to 5×.

The first part of hybridizing samples to the array used Hybridization Buffer (Table 5), which primarily consists of Saline-Sodium Citrate (SSC) (Table 4).

TABLE 4
20X Saline-Sodium Citrate.

| Ingredient | Amount |
|---|---|
| Sodium Chloride | 3M |
| Citric Acid, Trisodium Salt | 0.3M |

TABLE 5
Hybridization Buffer.

| Ingredient | Amount |
|---|---|
| 20X Saline-Sodium Citrate (SSC) | 2X to 5X |
| Bovine Serum Albumin (BSA) | 1% to 3% |
| Polyethylene Glycol Bisphenol A | 1% to 3% |
| Epichlorohydrin Copolymer (PEG-C) | |

After the Hybridization Buffer was made, gold-conjugated NeutrAvidin (“NAG”) was added to the Buffer and diluted to a final dilution of 0.05 OD to 0.2 OD. The amplicon produced by PCR (described above) was added to the Hybridization/Neutravidin Gold mixture such that 1 μl to 5 μl amplicon was present in every 210 μl of the mixture.

Once the samples were hybridized to the array (see Example 4), the array was washed with a Hybridization Wash Buffer (Table 6), which again included SSC buffer (Table 4). This Wash Buffer contained detergent in an amount sufficient to reduce background signal on the array.

TABLE 6
Hybridization Wash Buffer.

| Ingredient | Amount |
|---|---|
| 20X SSC | 1X to 5X |
| N-Lauroylsarcosine Sodium Salt | 0.05% to 2% |

### Results

A test of different concentrations of Empigen BB in the Lysis Buffer is illustrated in FIG. 3B. In this test, the target was a part of HIV, which is an RNA virus. Using Empigen BB promoted viral lysis, which released the RNA, and meant that the lysis product (lysate) could be used directly in RT-PCR or PCR. The asymmetric amplification system was used to amplify the HIV target. One complementary sequence was used, and one probe was dotted onto the array multiple times (dotted rectangles indicate location of probe on array in FIG. 3B) along with BSA-Gold positive controls (indicated by solid rectangles on right side in FIG. 3B). Empigen BB concentrations of 0% to 1% allowed target amplification, while concentrations of 2.5% to 10% inhibited target amplification.

### Example 4: 10-minute, 1-step Hybridization and Array Processing

The universal array concept described in Example 1 was tested using the following processing protocols. These protocols allow PCR or RT-PCR amplification products to be directly hybridized to arrays (i.e., without an extra clean-up step).

### Methods

Before hybridization, the array was blocked using 150 μl of 2% BSA in 1×PBS. The array was then placed in a thermoshaker at 37° C. and 250 RPM for 60 minutes. After blocking, the array was washed with 150 μl Ultrapure H₂O three times so that excess unbound reagents were removed. The final wash was left in the wells until the samples were ready in order to prevent the array from drying out.

To prepare the samples for hybridization, 2 ml of the Hybridization Buffer/NAG mixture (Hyb/NAG) were prepared per slide by adding 20 μl 10 OD NAG to 2 ml Hybridization Buffer (see Example 3 for details). Then, 0.5 ml microfuge tubes were prepared with 210 μl of Hyb/NAG for each amplicon, as each amplicon would be loaded into two wells and each well required 100 μl. After preparing the tubes, 2.1 μl of amplicon was added to each tube and briefly vortexed, after which all tubes were briefly centrifuged.

To hybridize, the final wash was removed from the wells, and 100 μl of Hyb/NAG/amplicon mix was added per well. Then, the array was placed in a thermoshaker at 37° C. and 250 RPM for 10 minutes.

After hybridization, the mix was pipetted out of the wells. Then, the array was washed with 100 μl of Hybridization Wash Buffer (see Example 3 for details) three times to remove unbound reagents Immediately after the third wash, the array was washed with 150 μl of Ultrapure H₂O three times. The final wash was left in the wells until the silver stain was ready in order to prevent the array from drying out.

The silver stain used was the kit made by Intuitive Biosciences. The slides were imaged using GenePix Pro 7.

### Results

The effect of different hybridization buffer formulations on stringency is shown in FIG. 3C. Here, the asymmetric amplification system was used to amplify an HCV target. One complementary sequence was used, and one probe was dotted onto the array multiple times along with BSA-Gold positive controls (indicated by solid rectangles in FIG. 3C). Two different Hybridization Buffer formulations were used to prepare the samples for hybridization in this test. The first formulation, which was used to prepare the samples in the top row of panels, consisted included 3×SSC, 1% BSA, and 3% PEG-C (3/1/3 Hybridization Buffer). The second formulation, which was used to prepare the samples in the bottom row of panels, included 2×SSC, 1% BSA, and 2% PEG-C (2/1/2 Hybridization Buffer). In comparing the two formulations, it can be seen that the 3/1/3 Hybridization Buffer is less stringent than the 2/1/2 Hybridization Buffer, because a signal is seen in the absence of lysate (compare dotted rectangles in top and bottom panels on the left) and non-specific signals are seen at different amounts of Lysate.

### Example 5: Two-Step Bead Enrichment Method to Prepare Samples

Before the steps described in Example 1 for nucleic acid testing using the universal array concept, magnetic beads can be used to enrich nucleic acids of interest from a sample. In this example, streptavidin-coated magnetic beads were labeled with biotin-labeled oligonucleotides complementary to the nucleic acid of interest. These beads were then added to a sample/Lysis Buffer mixture to bind the nucleic acid of interest. The nucleic acid of interest was then removed from the beads using sodium hydroxide, and the eluted target was neutralized using Tris.

### Methods

The following protocol was used to bind the biotin-labeled oligonucleotides to the magnetic beads.
1. Add 400 μl of streptavidin-coated magnetic beads (here: Bangs Lab beads (Cat #BM568) to a 1.5 ml tube.
2. Magnetically separate beads for 30 seconds to isolate beads, then carefully remove supernatant by pipetting and discard it.
3. Resuspend beads in 200 μl Binding Buffer (20 mM Tris pH 8.0/0.5M NaCl).
4. Add 41 of a biotin-labeled oligonucleotide (here: Biotinylated HIV reverse primer), then incubate the solution with rocking for 15 minutes at room temperature.
5. Magnetically separate beads for 30 seconds, then carefully remove supernatant by pipetting and discard it.
6. Wash beads using 200 μl Binding Buffer, while still on magnetic apparatus. Discard the buffer supernatant.
7. Repeat the wash again using 200 μl binding buffer. Discard the Buffer supernatant.
8. Resuspend the magnetic beads with biotin-primer bound in 200 μl Binding Buffer.

The following protocol was used to enrich the nucleic acid of interest from the sample.
1. Mix a sample (here: HIV RNA serum sample) 1:1 with Lysis Buffer (this mixture is called sample lysate). A total volume of 200 μl sample lysate is recommended.
2. Add 5 μl of streptavidin-coated magnetic beads labeled with biotin-labeled oligonucleotides to sample lysate.
3. Mix by pipetting and incubate for 10 minutes at room temperature.
4. Magnetically separate the beads for 60 seconds.
5. Carefully remove the supernatant by pipetting and discard it.
6. Wash the beads using 100 μl Binding Buffer.
7. Carefully remove the Binding Buffer supernatant and discard it.
8. Resuspend the magnetic beads in 5 μl of 0.1M sodium hydroxide (NaOH).
9. Incubate at room temp for 30 seconds.
10. Magnetically separate the beads for 15 seconds.
11. Carefully remove the supernatant (˜5 μl) and transfer to a fresh 1.5 ml tube containing 5 μl 100 mM Tris and mix by pipetting (Elution 1).
12. Resuspend the beads still in the original tube in 10 μl 100 mM Tris (Elution 2).

### Results

The effect of using different NaOH concentrations to remove the nucleic acid of interest from the magnetic beads is illustrated in FIG. 3D. Here, the sample lysate used as input was an HIV RNA serum sample diluted to 10⁵ copies per ml, and then mixed 1:1 with Lysis Buffer. Using concentrations of NaOH that are 0.05N and higher was seen to effectively remove the nucleic acid of interest from the beads (compare the signal within the rectangles of the bottom set of panels, labeled “Elution 1”, to the signal within the rectangles of the top set of panels, labeled “Beads after Elution” in FIG. 3D). In contrast, when no NaOH is used, the nucleic acid of interest remains attached to the beads (compare the signals within the rectangles of the top and bottom right-most panels in FIG. 3D).

The effect of using different NaOH concentrations on the signal strength of the subsequent RT-PCR is illustrated in FIG. 3E. Here, the sample lysate used as input was an HIV RNA serum sample diluted to 10³ copies per ml, and then mixed 1:1 with Lysis Buffer. Less than 0.1 N NaOH can be used if 5 μl of the enriched nucleic acid of interest are used as RT-PCR input, but at least 0.1 N NaOH is required if 3 μl of the enriched nucleic acid of interest are used as RT-PCR input (compare the signal within the rectangles of the top and bottom sets of panels in FIG. 3E).

A comparison between different methods of eluting the enriched nucleic acid of interest is illustrated in FIG. 3F. Here, the sample lysate used as input was an HIV RNA serum sample diluted to 10³ copies per ml, and then mixed 1:1 with Lysis Buffer. Using 100 mM Tris to elute the enriched nucleic acid of interest from the magnetic beads resulted in a stronger downstream signal than using 10 mM TE, regardless of whether 3 μl or 5 μl were used in the subsequent RT-PCR (compare the signal within the blue rectangles of the top and bottom sets of panels in FIG. 3F).

### Example 6: One-Step Bead Enrichment Method to Prepare Samples

Before nucleic acid testing using the universal array concept described in Example 1, magnetic beads can be used to enrich nucleic acids of interest. In this example, streptavidin-coated magnetic beads are mixed with biotin-labeled oligonucleotides complementary to the nucleic acid of interest as well as the sample lysate. Thus, the hybridization of the oligonucleotide to the nucleic acid of interest occurs in the same step as binding of the biotin-labeled oligonucleotide to the magnetic beads.

### Method

The following protocol was used in the one-step bead enrichment method.
1. Add 40 μl of magnetic beads (here: Nvigen beads (Cat #K61002)) to a 1.5 ml tube.
2. Using a magnetic apparatus, magnetically separate beads for 30 seconds to isolate beads, then carefully remove supernatant by pipetting and discard it.
3. While the tube is still on the magnetic apparatus, wash beads using 200 μl of Binding Buffer, then discard the Buffer supernatant.
4. Remove the tube from the magnetic apparatus and resuspend the unlabeled magnetic beads in 200 μl Binding Buffer (20 mM Tris/0.5M NaCl).
5. Prepare 1× Lysis Buffer (1×PBS/1% Empigen BB) with biotin-labeled oligonucleotide (here: biotin HIV-R3 primer).
a. Recommended concentrations are 5 picomole (pM), 25 pM, or 125 pM primer per 100 μl Lysis Buffer A.
6. Dilute sample (here: HIV serum) to preferred concentration in healthy plasma or Dilution Buffer (10 mM Tris/0.1 mM EDTA) in a 1.5 mL tube. A final volume of 100 μl diluted sample is recommended.
7. Add Lysis Buffer that contains primer at a 1:1 ratio to diluted sample from step 6 to produce sample lysate. A final volume of 200 μl of sample lysate is recommended.
8. Incubate mixture at room temperature for 10 minutes to allow primer to bind nucleic acid of interest in lysed sample.
9. Add 5 μl unlabeled magnetic beads (from step 4) to mixture from step 8.
10. Mix by pipetting, then incubate for 5 minutes at room temperature.
11. Mix by pipetting a second time, and then incubate for an additional 5 minutes at room temperature.
12. Using a magnetic apparatus, magnetically separate the beads for 60 seconds, then carefully remove the supernatant by pipetting and discard it.
13. Wash the beads using 100 μl Binding Buffer, then carefully remove the binding buffer supernatant and discard it.
14. Remove the tube from the magnetic apparatus and resuspend the beads in 5 μl of 0.1 M sodium hydroxide (NaOH).
15. Incubate at room temp for 30 seconds.
16. Using the magnetic apparatus, magnetically separate the beads for 15 seconds.
17. Carefully remove the supernatant (˜5 μl) and transfer to a fresh 1.5 mL tube containing 5 μl 100 mM Tris and mix by pipetting (Elution 1)
18. Resuspend the beads still in the original tube in 10 μl 100 mM Tris (Elution 2).

### Results

The effect of using different biotin-labels oligonucleotide (primer) concentrations in one-step enrichment is illustrated in FIG. 3G. Here, the input sample was an HIV RNA serum sample diluted to 10⁵ copies per ml, which was compared with a negative control. Primer concentrations ranging from 5 pM to 125 pM were effective when used in one-step enrichment (compare the signal within the rectangles of the bottom set of panels, labeled to the signal within the rectangles of the top set of panels in FIG. 3G). Moreover, one-step enrichment was more effective than two-step enrichment (compare the signals within the rectangle of the bottom right-most panel to the other panels on the bottom of FIG. 3G).

### Example 7: Anchored Filters in Pipette Tips

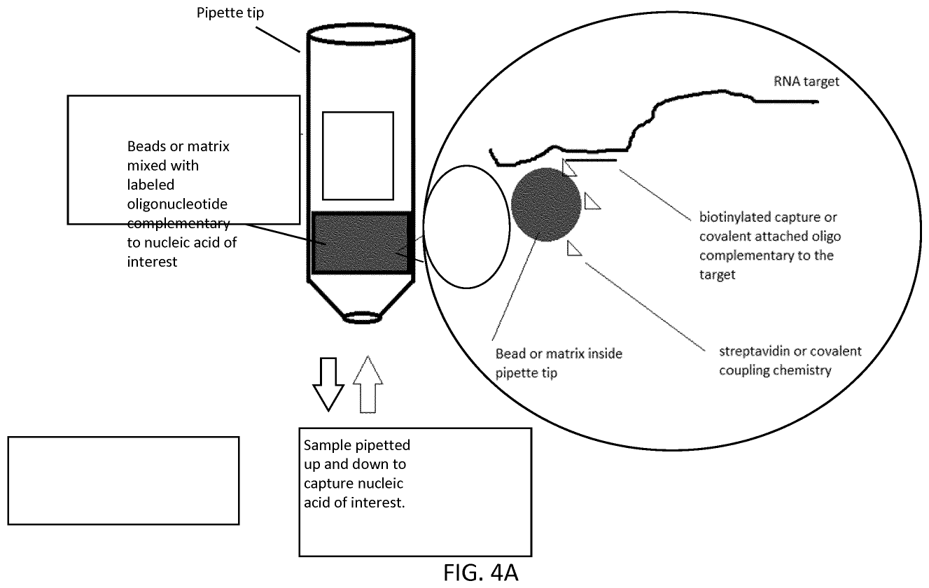

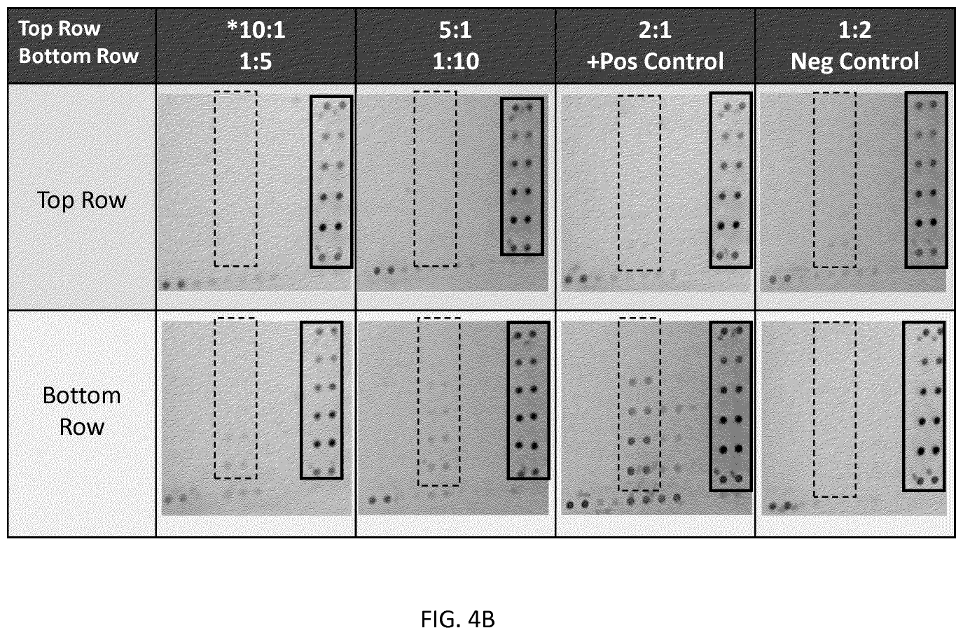

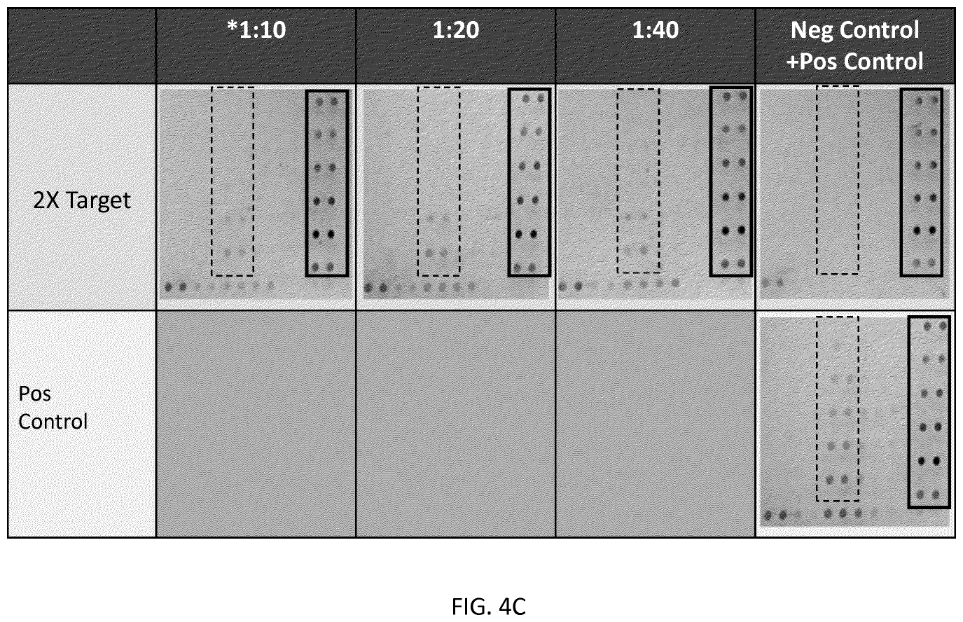

Sample enrichment in a pipette tip is illustrated in FIG. 4A. Here, an anchored filter retains magnetic beads or a matrix with covalent coupling chemistry (e.g., streptavidin) inside the tip. In addition, the tip contains oligonucleotides able to be attached to the beads or matrix (e.g., via biotin) that are complementary to the nucleic acid of interest (see Examples 5 and 6 for example embodiments of sample enrichment processes).

First, the sample is pipetted up and down through the pipette tip, whereby the nucleic acid of interest is captured by the oligonucleotide. Then, wash buffers are pipetted up and down through the pipette tip to remove any excess reagents or sample. Finally, elution buffer is pipetted up and down through the tip to elute the nucleic acid of interest.

The nucleic acid of interest can subsequently be used in the universal array concept described in Example 1.

### Example 8: Ratio of Biotin to Neutravidin Gold

The ratio of biotin-labeled oligonucleotide to neutravidin-labeled colloidal gold is important for signal detection when using a universal array as described in Example 1. This concept is illustrated in FIGS. 4B & 4C. As with previous experiments (see Example 4), one complementary sequence was used and one probe was spotted onto the array multiple times along with BSA-Gold positive controls (indicated by solid rectangles). FIG. 4B used a 1× Target concentration and FIG. 4C twice the target amount as in FIG. 4B. Target complementary to the spotted probe was incubated and then washed after which a mixture of Biotin-labeled probe complementary to the opposite end of the target bound to the array and Neutravidin-labeled colloidal Gold (NAG) in a range of ratios (Biotin Probe: NAG) was added to the slide, incubated, washed, and silver enhanced. On both FIGS. 4B & 4C, decreasing the ratio of Biotin-labeled probe to NAG increased signal in a 1-Step detection assay (dotted rectangles). The Positive Control was an example of a 2-Step detection assay where target was incubated with the array, washed away, then Biotin-labeled probe complementary to the target was added, excess washed away prior to adding NAG.

### Example 9: Continuous Amplification System

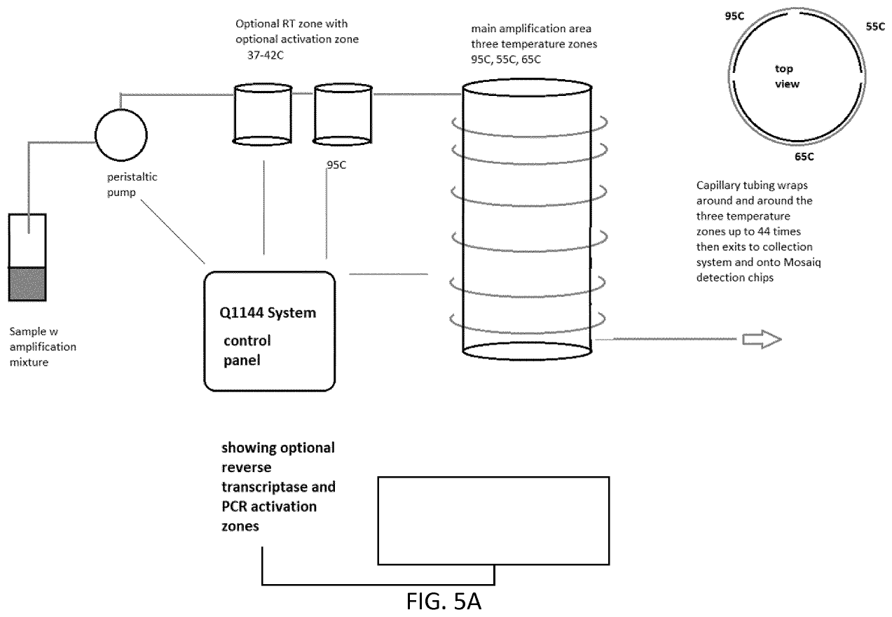

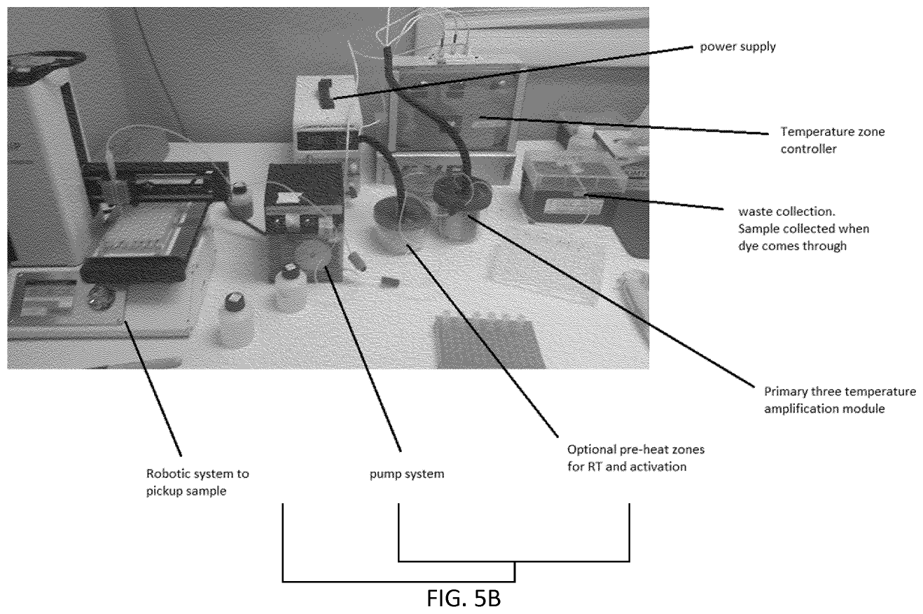

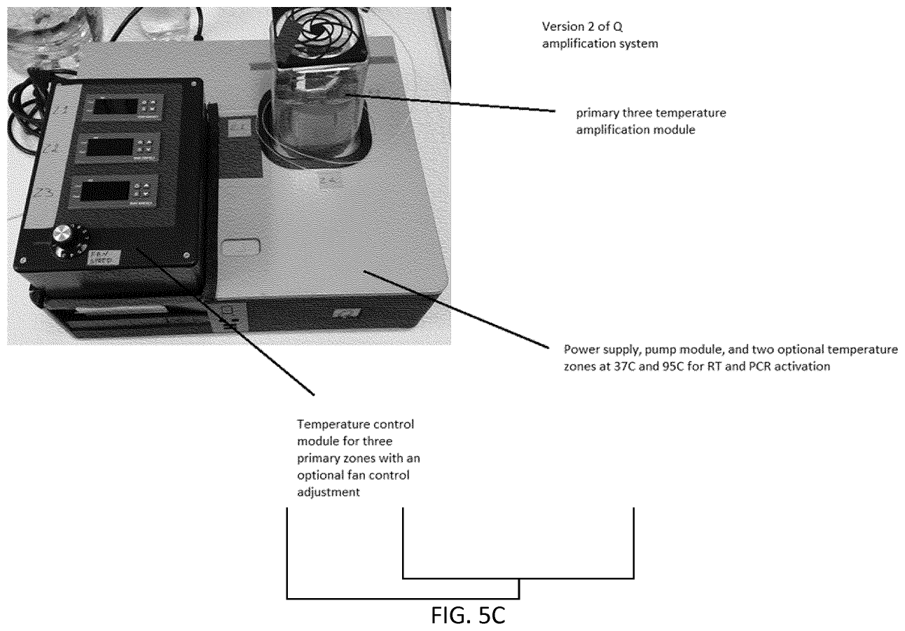

A continuous amplification system using capillary tubing can be used to amplify nucleic acids of interest. This concept is illustrated in FIG. 5A. First, a peristaltic pump is used to move the sample mixed with the amplification (e.g., PCR) mixture out of the initial container and into the capillary tubing. Then, it passes through an optional RT zone (necessary if the sample is RNA), which is held at a constant temperature (e.g., 37° C. or 42° C.). The sample is typically kept in this zone for 15 minutes (e.g., 15 loops of the capillary tubing if a circular heating system is used).

Next, the sample passes through an optional PCR activation zone, which is held at a constant temperature (e.g., 95′C). The sample is typically kept in this zone for 10 minutes to activate the PCR reaction components (e.g., 10 loops of the capillary tubing if a circular heating system is used). After these two optional areas, the sample reaches the main amplification area, which is a cylinder that is vertically divided (see top view in FIG. 5A) into three constant temperature zones (e.g., 95° C., 55° C., 65° C.). Each of these zones corresponds to one portion of a standard PCR reaction, namely denaturing, annealing, and extension. The capillary is wrapped around the main amplification cylinder multiple times (e.g., 44 wraps), so that as the sample passes through the loops, it goes repeatedly through each of the three zones in sequence. The divisions are proportioned such that the sample spends about 15 seconds in the first zone, 15 seconds in the second zone, and 30 seconds in the third zone.

Finally, the sample exits the amplification system and enters the collection system where it is detected (e.g., MosaiQ detection chips). Indicator dyes within the amplification mixture are used to trigger the collection processes.

The pump, RT zone, PCR activation zone, and main amplification area are all controlled by a system control panel. This allows alteration of each of the components, for example the temperature of the different zones can be changed. In addition, the control panel can be used to change the pumping speed, thereby altering the length of time spent in each temperature zone of the main amplification area.

One example of a continuous amplification system is illustrated in FIG. 5B. This unit uses a robotic arm and a peristaltic (or HPLC) pump to pick up a sample that has already been extracted and added to an RT-PCR mix, and move the sample into capillary tubing. Then, the pump delivers the sample via capillary tubing to an optional preheat zone (adjustable but set to 37° C.) for 10-15 minutes, and after that the sample is delivered to an optional PCR activation zone (adjustable but set to 95° C.) for 10 minutes. Subsequently, the sample is delivered to the PCR amplification module, where it cycles for about one minute per wrap of the capillary tube around three stationary, but adjustable, temperature zones. These zones allow the sample to be heated to 95° C. for about 15 seconds, then heated to about 55° C. to allow primers to anneal for about 15 seconds, then onto the amplification/extension zone of 65-72° C. where the amplification occurs, which then loops back to the denaturing zone of 95° C. for the next loop. This continues for 40-50 loops (cycles) of the capillary tube until the sample exits the amplification module and passes over an optional 72° C. extension zone for 5 minutes and into the collection tray, which detects the amplified product as it contains a dye (such as blue or FITC).

A second example embodiment of a continuous amplification system is illustrated in FIG. 5C. This example included the “Q coil system” (visible in the upper right of the image encased in a vented, clear, plastic encasement, which contains the 3 temperature zones in the coil) together incorporated with the power supply (not visible, internal), case fan with analog control for more accurate temperature control vs. no fan (such as the Q1000 as seen in FIG. 5B), and 3 digital, programmable displays (bottom left). A continuous tubing, which is pumped externally (not visible here) by peristaltic pump (or other pump mechanism such as HPLC) from the robotic sample plate collection apparatus (not shown), flows in to the back of the Q1144 Case, then through the Q-coil system encasement (which contains the 3 temperature zones of Z1=95′C, Z2=55′C, and Z3=65′C), and finally exits the Q114 Case for final dispensing & collection into sample tubes. This system holds the 3 temperature zones most consistently & gave results consistently down to 10\hat{ }4 copies/mL of Serum Positive (1 uL sample/reaction tube mixture=˜10 HIV DNA copies).

Methods

The reaction mixture and protocols used to detect HIV in a continuous amplification system (see FIGS. 5A-5C) are provided below.

Per Reaction Master Mix (24 ul/rxn):
7 μl nuclease-free water
12.5 μl BIOTIUM mix (with Eva green in it)
1.5 μl 5M Betaine buffer
1 μl0.2% blue dye
0.75 μl forward primer (Q-HIV-F1-tc, 300 nM)
0.75 μl reverse primer (Biotin-HIV-R3, 300 nM)

### 0.5 μl Promega RT mix

- - -
24 μl master mix per tube, +1 μl sample.

Samples were extracted by 1:1 dilution in Lysis buffer “X1”. The Lysis buffer X1 formulation is [2% Triton X100+1×PBS]. An alternative Lysis Buffer is “A1”=[2% Empigen BB+1×PBS], which works well in both the PCR system and Q system, however, Lysis-X1 buffer performed somewhat better in the Q system and was used for final prototype testing. Samples were extracted for 10 minutes prior to use.

Primers were used at 10 μM (diluted fresh from 100 μM stock before use).

### For Sample Loading:

The robotic arm dipped the inlet to the peristaltic pump into different wells or columns. Each column corresponded to one test, and each test used different amounts of water washes, bleach, and neutralization chemicals. The system used small air gaps between disinfection and wash steps. The air, bleach, and wash steps were as follows:

I. Prepare 25 μl of sample and load each into plate in row 1 (Note: columns 1 and 12 are water only, and 200 μl were loaded in each well for both columns). All water used was nuclease-free water.
II. Prep 100 μl of each of the other rows as follows:
20% bleach (1.6% final) in row 3, columns 2-11.
20 mM Thiosulfate in row 5, columns 2-11.
Water in rows 6, 7, 8 . . . columns 2-11 each.

Sample Loading Program:

1. Collect sample from row 1 for 60 s. Air for 30 s.
2. 2× bleach pulses for 20 s each from row 3 (approximately 15 μl each), 20 s air in between.
3. 2× thiosulfate pulses for 20 s each from row 5 (approximately 15 μl each), 20 s air in between.
4. 1× water pulse from row 6 for 15 s, 15 s air.
5. 1× water pulse from row 7 for 15 s, 15 s air.
6. 2× water pulse from row 8 for 15 s, 15 s air.
7. Delay 30 s (air) before next sample collection.

Bleach was used to clear the amplicons from the lines before the next sample was introduced. Because bleach carryover would inactivate the next reaction, various methods to inactivate the bleach were tested. These included: water dilution, air pockets, sodium meta-bisulfite, sodium, and potassium thiosulfate. By using these methods, the system did not need to be extensively rinsed between samples. Thus, it was possible to perform multiple amplifications (e.g., of different targets) in succession. Many iterations of washes were used and tried, however, water alone was not sufficient to clear the previous samples a short amount of time (it took over 10 minutes of water pulses to avoid carry-over), while bleach alone was too strong without sufficient washes of water (and eventually discovered thiosulfate worked best to neutralize the bleach quickly), with optimal washes noted in section 140. Bleach used was 20% of 8.2% hypochlorite stock (approximately 1.6% final), while thiosulfate was 20 mM final, for the washes listed in the program above.

### Example 10: Asymmetric Amplification

The asymmetric amplification concept employs a skewed primer ratio to achieve a predominantly uniform product. Asymmetric amplification is the second of the two methods described herein that can be used to prepare samples for hybridization to the universal array (see Example 1). Asymmetric amplification can be used in thermocycling amplification processes, such as polymerase chain reaction (PCR), or in isothermal amplification processes, such as recombinase polymerase amplification (RPA).

Asymmetric amplification uses two primers, namely a forward primer (also known as a 5′ primer or a sense primer) and a reverse primer (also known as a 3′ primer or an antisense primer). The forward primer has the same sequence as the capture on the universal array as well as a short portion of the 5′ end of the nucleic acid of interest. The reverse primer is specific for the nucleic acid of interest (in this case, the 3′ end of the nucleic acid of interest) and has a detectable label (in this case, biotin at its 5′ end) to allow detection of the amplicon hybridized to the array. The reverse primer is used in an amount that is in excess of the amount used for the forward primer. For example, 15-20 nM of the reverse primer and 5-10 nM of the forward primer can be used for a 2:1 or 3:1 ratio. The excess can also be larger, for example a ratio of reverse to forward primer from 12.5:1 to 100:1 can be used, e.g., 20:1. This skewed primer ratio results in the forward primer being used up before the reverse primer during the amplification process. Thus, the primary product in the final solution is that produced by the reverse primer.

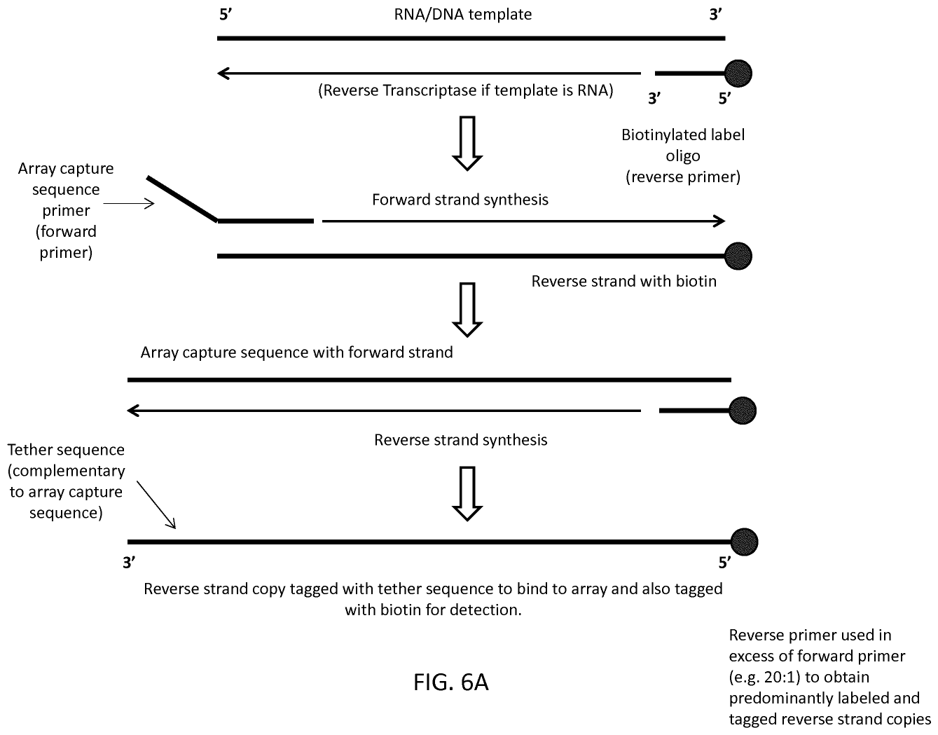

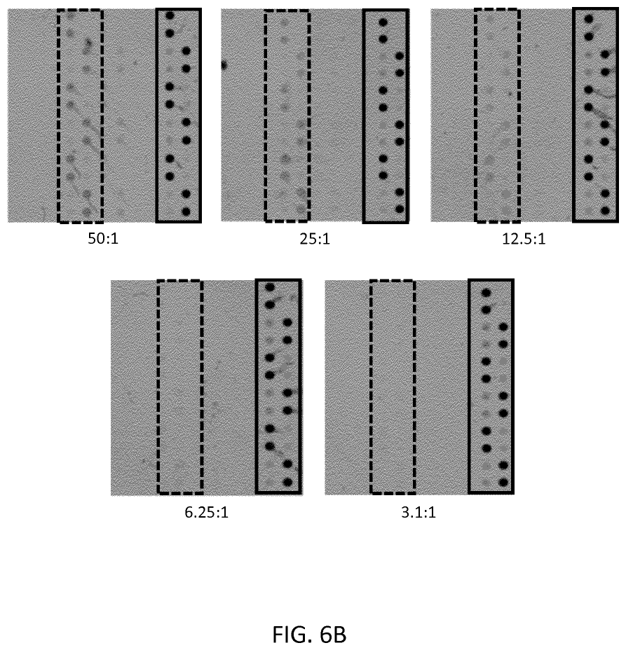

A diagram showing the steps of the asymmetric RPA process is shown in FIG. 6A. RPA allows the use of either a DNA or an RNA template by including reverse transcriptase to directly produce a DNA strand from an RNA template (first step shown in FIG. 6A). If a DNA template is being used, the reverse strand is already present, and so does not need to be synthesized. The asymmetric amplification process begins with this reverse strand.

Once a reverse strand is present, the forward primer binds to it and the forward strand is synthesized (second step shown in FIG. 6A). Because the forward primer contains the universal array capture sequence, the synthesized forward strand now contains the universal array capture sequence at the 5′ end of the template sequence.

In the next step, the reverse primer binds to the synthesized forward strand and the reverse strand is synthesized (third step shown in FIG. 6A). At the 3′ end, the tether sequence, which is complementary to the universal array capture sequence, is synthesized. The synthesized reverse strand therefore contains (from 3′ to 5′) a tether sequence, a reverse strand copy of the template, and a biotin tag (product shown in FIG. 6A). Because the reverse primer is used in excess, the result of asymmetric PCR is predominantly this synthesized reverse strand. The synthesized reverse strand can then be hybridized to the universal array (using the tether sequence), and detected (using the biotin).

A test of different ratios of reverse primers to forward primers is shown in FIG. 6B. In this example, primers designed to an HCV target conjugated with one complementary sequence. One probe was dotted onto the array multiple times (dotted rectangles indicate location of probe on array in FIG. 6B) along with BSA-Gold positive controls (indicated by solid rectangles in FIG. 6B). The strength of the signal seen on the array increases as the amount of excess reverse primer increases.

Although the foregoing disclosure has been described in some detail by way of illustration and example for purposes of clarity of understanding, the descriptions and examples should not be construed as limiting the scope of the present disclosure. The disclosures of all patent and scientific literature cited herein are expressly incorporated in their entirety by reference.

What is claimed is:

1. A method for detecting a nucleic acid in a sample, comprising:
a) amplifying at least a portion of a nucleic acid from a sample using a primer pair under conditions suitable for amplification of an amplicon comprising the portion of the nucleic acid if present in the sample, wherein the primer pair comprises:
1) a first primer comprising a label and a first oligonucleotide sequence that hybridizes with a first strand of the portion of the nucleic acid, and
2) a second primer comprising a second oligonucleotide sequence that hybridizes with a second strand of the portion of the nucleic acid opposite the first strand and a third oligonucleotide sequence;
b) after step (a), contacting the amplicon, if present, to a plurality of single-stranded oligonucleotide capture sequences each affixed to a solid support, and wherein the amplicon, if present, hybridizes with at least one of the single-stranded oligonucleotide capture sequences on its solid support via the third oligonucleotide sequence or the complement of the third oligonucleotide sequence;
c) after step (a), applying a colloidal detection reagent to the solid supports, wherein the colloidal detection reagent comprises a first moiety that binds to the label of the amplicon if present and a second moiety that comprises a colloidal metal;
d) after (c), washing the solid supports with a wash solution; and
e) after steps (a)-(d), detecting the colloidal detection reagent, wherein detection of the colloidal detection reagent on a solid support indicates the presence of the hybridized amplicon, thereby detecting the nucleic acid in the sample.

2. The method of claim 1, wherein the solid supports are arranged as a microarray, a multiplex bead array, or a well array.

3. The method of claim 1, wherein the solid supports are nitrocellulose, silica, plastic, or hydrogel.

4. The method of claim 1, wherein detecting the colloidal detection reagent in step (e) comprises detection of the colloidal metal.

5. The method of claim 1, wherein detecting the colloidal detection reagent in step (e) comprises:
1) applying a developing reagent to the solid supports, wherein the developing agent is suitable for forming a precipitate in the presence of the colloidal metal; and
2) detecting the colloidal detection reagent by detecting the formation of the precipitate on a solid support.

6. The method of claim 5, wherein the formation of the precipitate is detected by visual, electronic, or magnetic detection.

7. The method of claim 5 or claim 6, wherein the formation of the precipitate is detected by a mechanical reader.

8. The method of any one of claims 5-7, wherein the developing reagent comprises silver.

9. The method of any one of claims 1-8, wherein the conditions in step (a) are suitable for amplification by polymerase chain reaction (PCR).

10. The method of any one of claims 1-8, wherein the conditions in step (a) are suitable for amplification by recombinase-polymerase assay (RPA), nucleic acid sequenced-based chain assay (NASBA), rolling circle amplification, branched chain amplification, ligation amplification, or loop-mediated isothermal amplification.

11. The method of any one of claims 1-10, wherein the label comprises biotin and the third oligonucleotide sequence hybridizes with at least one of the single-stranded oligonucleotide capture sequences.

12. The method of any one of claims 1-11, wherein each single-stranded oligonucleotide capture sequence is coupled to a spacer reagent, and the spacer reagent is coupled to the corresponding solid support.

13. The method of claim 12, wherein the spacer reagent comprises a serum albumin protein.

14. The method of claim 12, wherein the spacer reagent comprises a dendrimer.

15. The method of any one of claims 1-14, further comprising washing the solid supports with a wash solution after step (b).

16. The method of any one of claims 1-15, wherein the first primer is a forward primer that amplifies in the sense direction of the nucleic acid, and the second primer is a reverse primer that amplifies in the antisense direction of the nucleic acid.

17. The method of any one of claims 1-15, wherein the second primer is a forward primer that amplifies in the sense direction of the nucleic acid, and the first primer is a reverse primer that amplifies in the antisense direction of the nucleic acid.

18. The method of any one of claims 1-17, wherein the second primer comprises:
the second oligonucleotide sequence, wherein the second oligonucleotide sequence allows for primer extension in the 5′ to 3′ direction; and
the third oligonucleotide sequence, wherein the third oligonucleotide sequence is oriented in the opposite 5′ to 3′ direction compared with the direction of primer extension from the second oligonucleotide sequence.

19. The method of claim 18, wherein the third oligonucleotide sequence comprises a modified nucleotide at the 3′ terminus that blocks primer extension.

20. The method of claim 18 or claim 19, wherein the second primer further comprises one or more linkers between the 5′ end of the third oligonucleotide sequence and the 5′ end of the second oligonucleotide sequence.

21. The method of any one of claims 1-17, wherein the portion of the nucleic acid is amplified in step (a) using an excess of the first primer relative to the second primer, and wherein the amplicon, if present, is a single-stranded nucleic acid that hybridizes with at least one of the single-stranded oligonucleotide capture sequences in step (b) via the complement of the third oligonucleotide sequence.

22. The method of claim 21, wherein the portion of the nucleic acid is amplified in step (a) using a ratio of first primer to the second primer of between about 12.5:1 and about 100:1.

23. The method of any one of claims 1-22, wherein the label of the first primer comprises biotin.

24. The method of claim 23, wherein the first moiety of the colloidal detection reagent comprises neutravidin, streptavidin, or an antigen-binding domain that specifically binds biotin.

25. The method of claim 24, wherein the first moiety of the colloidal detection reagent comprises neutravidin, and wherein the second moiety of the colloidal detection reagent comprises a colloidal gold ion.

26. The method of any one of claims 1-25, wherein the colloidal detection reagent is applied to the solid supports in step (c) at a final dilution of 0.00001OD to 20OD.

27. The method of claim 26, wherein the first moiety of the colloidal detection reagent comprises neutravidin, wherein the second moiety of the colloidal detection reagent comprises a colloidal gold ion, and wherein the colloidal detection reagent is applied to the solid supports in step (c) at a final dilution of 0.05OD to 0.2OD.

28. The method of claim 27, wherein 1 pL to 1000 μL of colloidal detection reagent is applied to the solid supports in step (c) per μL of amplicon.

29. The method of any one of claims 1-28, further comprising, prior to step (a), exposing the sample to a lysis buffer comprising greater than or equal to 0.1% and less than or equal to 10% N,N-dimethyl-N-dodecylglycine betaine (w/v).

30. The method of claim 29, wherein the lysis buffer comprises greater than or equal to 0.5% and less than or equal to 4% N,N-dimethyl-N-dodecylglycine betaine (w/v).

31. The method of claim 29, wherein the lysis buffer comprises greater than or equal to 1% and less than or equal to 2% N,N-dimethyl-N-dodecylglycine betaine (w/v).

32. The method of any one of claims 29-31, wherein the sample is exposed to the lysis buffer at a ratio between 1:50 sample:lysis buffer and 50:1 sample:lysis.

33. The method of claim 32, wherein the portion of the sample is exposed to the lysis buffer at a ratio of about 1:1 sample:lysis buffer.

34. The method of any one of claims 29-33, wherein the lysis buffer further comprises 0.1× to 5× phosphate buffered saline (PBS) buffer or Tris EDTA (TE) buffer.

35. The method of claim 34, wherein the lysis buffer further comprises 1×PBS.

36. The method of any one of claims 1-35, wherein the amplicon is hybridized to the solid supports in step (b) in a hybridization buffer comprising 0.1× to 10× saline sodium citrate (SSC) buffer, 0.001% to 30% blocking agent, and 0.01% to 30% crowding agent.

37. The method of claim 36, wherein the blocking agent comprises bovine serum albumin (BSA), polyethylene glycol (PEG), casein, or polyvinyl alcohol (PVA).

38. The method of claim 37, wherein the blocking agent comprises BSA, and the BSA is present in the hybridization buffer at 1% to 3%.

39. The method of any one of claims 36-38, wherein the crowding agent is Polyethylene Glycol Bisphenol A Epichlorohydrin Copolymer.

40. The method of claim 39, wherein the Polyethylene Glycol Bisphenol A Epichlorohydrin Copolymer is present in the hybridization buffer at 1% to 3%.

41. The method of any one of claims 36-40, wherein the hybridization buffer comprises 2× to 5×SSC buffer.

42. The method of any one of claims 1-41, further comprising, prior to step (b), blocking the solid supports using a solution comprising BSA.

43. The method of claim 41, wherein the solid supports are blocked for 1 hour at 37° C. using 2% BSA solution.

44. The method of claim 41 or claim 43, further comprising washing the solid supports with a wash solution after blocking the solid supports.

45. The method of any one of claims 1-44, further comprising, after step (b) and prior to step (c), washing the solid supports with a wash buffer comprising 0.1× to 10×SSC buffer and 0.01% to 30% detergent.

46. The method of claim 45, wherein the detergent comprises 0.05% to 5% N-lauroylsarcosine sodium salt.

47. The method of claim 45 or claim 46, wherein the wash buffer comprises 1× to 5×SSC buffer.

48. The method of any one of claims 29-47, wherein one or more of the lysis buffer, wash buffer, and hybridization buffer further comprises a control oligonucleotide that hybridizes with at least one of the single-stranded oligonucleotide capture sequences on its solid support.

49. The method of any one of claims 1-48, further comprising, prior to step (a):
(i) contacting the sample with an oligonucleotide coupled to a solid substrate, wherein the oligonucleotide hybridizes with the nucleic acid if present in the sample;
(ii) washing the solid substrate under conditions suitable to remove non-specific interactions with the solid substrate but retain the nucleic acid hybridized with the oligonucleotide, if present in the sample; and
(iii) eluting the nucleic acid, if present in the sample, from the oligonucleotide, wherein the eluted nucleic acid is subjected to PCR amplification in step (a).

50. The method of any one of claims 1-48, further comprising, prior to step (a):
(i) contacting the sample with an oligonucleotide, wherein the oligonucleotide hybridizes with the nucleic acid if present in the sample,
(ii) simultaneous with or after step (i), contacting the sample with a solid substrate, wherein the solid substrate is coupled to a first binding moiety, wherein the oligonucleotide is coupled to a second binding moiety that binds the first binding moiety, and wherein the sample is contacted with the solid substrate under conditions suitable for the second binding moiety to bind the first binding moiety;
(iii) washing the solid substrate under conditions suitable to remove non-specific interactions with the solid substrate but retain the oligonucleotide and the nucleic acid hybridized with the oligonucleotide, if present in the sample; and
(iv) eluting the nucleic acid, if present in the sample, from the oligonucleotide, wherein the eluted nucleic acid is subjected to PCR amplification in step (a).

51. The method of claim 49, wherein the oligonucleotide is coupled to the solid substrate via a covalent interaction.

52. The method of claim 49 or claim 50, wherein the oligonucleotide is coupled to the solid substrate via an avidin:biotin or streptavidin:biotin interaction, or wherein the first binding moiety comprises avidin, neutravidin, streptavidin, or a derivative thereof and the second binding moiety comprises biotin or a derivative thereof.

53. The method of any one of claims 48-52, wherein the solid substrate is positioned in a pipet tip, and wherein step (i) comprises pipetting the sample into the pipet tip.

54. The method of any one of claims 48-53, wherein the solid substrate comprises a matrix or plurality of beads.

55. The method of any one of claims 1-54, wherein the nucleic acid comprises DNA.

56. The method of any one of claims 1-54, wherein the nucleic acid comprises RNA.

57. The method of claim 56, further comprising, prior to step (a), incubating at least a portion of the sample with a reverse transcriptase, primers, and deoxyribonucleotides under conditions suitable for generation of a cDNA synthesized from the nucleic acid, wherein the portion of the nucleic acid is amplified in step (a) using the cDNA.

58. The method of claim 57, wherein the primers used prior to step (a) are random primers, poly-dT primers, or primers specific for the portion of the nucleic acid.

59. The method of claim 57 or claim 58, wherein the portion of the sample is incubated with the reverse transcriptase, primers, and the deoxyribonucleotides in the presence of an RNase inhibitor.

60. The method of any one of claims 57-59, wherein the portion of the sample is incubated with the reverse transcriptase, primers, and the deoxyribonucleotides in the presence of betaine.

61. The method of claim 60, wherein the betaine is present at a concentration of about 0.2M to about 1.5M.

62. The method of any one of claims 1-61, wherein the nucleic acid comprises a viral nucleic acid.

63. The method of claim 62, wherein the viral nucleic acid is from a virus selected from the group consisting of HIV, HBV, HCV, West Nile, Zika, and parvovirus.

64. The method of any one of claims 1-61, wherein the nucleic acid comprises a bacterial, archaean, protozoan, fungal, plant, or animal nucleic acid.

65. A kit, comprising: a plurality of primer pairs, wherein each primer pair of the plurality comprises a first primer coupled to a label, wherein the first primer hybridizes with a first strand of a nucleic acid, and a second primer comprising:
1) a first oligonucleotide sequence that allows for primer extension in the 5′ to 3′ direction and hybridizes with a second strand of the nucleic acid opposite the first strand;
2) a second oligonucleotide sequence, wherein the second oligonucleotide sequence is oriented in the opposite 5′ to 3′ direction compared with the direction of primer extension from the second oligonucleotide sequence; and
3) one or more linkers between the 5′ end of the first oligonucleotide sequence and the 5′ end of the second oligonucleotide sequence.

66. The kit of claim 65, wherein the second oligonucleotide sequence comprises a modified nucleotide at the 3′ terminus that blocks primer extension.

67. The kit of claim 65 or claim 66, wherein the label coupled to the first primer comprises biotin.

68. The kit of any one of claims 65-67, further comprising a plurality of single-stranded oligonucleotide capture sequences each affixed to a solid support, and wherein at least one single-stranded oligonucleotide sequence on its solid support hybridizes with the second oligonucleotide sequence of a second primer of a primer pair of the plurality.

69. A kit, comprising:
a) a plurality of single-stranded oligonucleotide capture sequences each affixed to a solid support; and
b) a plurality of primer pairs, wherein each primer pair of the plurality comprises:
1) a first primer comprising a label and a first oligonucleotide sequence that hybridizes with a first strand of the portion of the nucleic acid, and
2) a second primer comprising a second oligonucleotide sequence that hybridizes with a second strand of the portion of the nucleic acid opposite the first strand and a third oligonucleotide sequence, wherein the third oligonucleotide sequence of each primer pair of the plurality hybridizes with a single-stranded oligonucleotide capture sequence on its solid support.

70. The kit of claim 69, wherein the second oligonucleotide sequence of each primer pair of the plurality allows for primer extension in the 5′ to 3′ direction, wherein the third oligonucleotide sequence of each primer pair of the plurality is oriented in the opposite 5′ to 3′ direction compared with the direction of primer extension from the second oligonucleotide sequence, and wherein the second primer of each primer pair of the plurality further comprises one or more linkers between the 5′ end of the third oligonucleotide sequence and the 5′ end of the second oligonucleotide sequence.

71. The kit of claim 70, wherein the third oligonucleotide sequence of each primer pair of the plurality comprises a modified nucleotide at the 3′ terminus that blocks primer extension.

72. The kit of any one of claims 69-71, wherein each of the single-stranded oligonucleotide capture sequences on its support is coupled to a spacer reagent, and the spacer reagent is coupled to the solid support.

73. The kit of claim 72, wherein the spacer reagent comprises a serum albumin protein.

74. The kit of claim 72, wherein the spacer reagent comprises a dendrimer.

75. A method for amplifying and detecting a nucleic acid in a sample, the method comprising:
a) incubating at least a portion of the sample with an amplification mixture comprising deoxyribonucleotides, a polymerase, and a primer pair, wherein the primer pair comprises a first primer comprising a label and a first oligonucleotide sequence that hybridizes with a first strand of a portion of the nucleic acid, and a second primer comprising a second oligonucleotide sequence that hybridizes with a second strand of the portion of the nucleic acid opposite the first strand and a first capture moiety;
b) passing the portion of the sample in admixture with the amplification mixture through first, second, and third stationary temperature zones for a plurality of cycles through continuous capillary tubing under conditions suitable for amplification of an amplicon comprising the portion of the nucleic acid, if present in the sample, wherein each cycle of the plurality comprises:
1) passing the portion of the sample in admixture with the amplification mixture through the first stationary temperature zone via the continuous capillary tubing at a first temperature and for a first duration suitable for denaturing the strands of the nucleic acid, if present in the sample,
2) after step (b)(1), passing the portion of the sample in admixture with the amplification mixture through the second stationary temperature zone via the continuous capillary tubing at a second temperature and for a second duration suitable for annealing the first and second primers to the respective strands of the nucleic acid, if present in the sample, and
3) after step (b)(2), passing the portion of the sample in admixture with the amplification mixture through the third stationary temperature zone via the continuous capillary tubing at a third temperature and for a third duration suitable for amplifying the nucleic acid target, if present in the sample, via the polymerase and primer pair;
c) after the plurality of cycles, associating the amplicon, if present in the sample, with a first capture moiety affixed to a solid support; and
d) detecting association of the amplicon, if present in the sample, with the solid support, wherein association of the amplicon with the one or more solid supports indicates the presence of the nucleic acid in the sample.

76. The method of claim 75, wherein the first capture moiety comprises a third oligonucleotide sequence, and wherein the second capture moiety comprises a single-stranded oligonucleotide capture sequence that hybridizes with the third oligonucleotide sequence or the complement of the third oligonucleotide sequence in step (c).

77. The method of claim 75 or claim 76, wherein detecting association of the amplicon, if present, with the solid support comprises:
i) applying a colloidal detection reagent to the solid support, wherein the colloidal detection reagent comprises a first moiety that binds to the label of the amplicon if present and a second moiety that comprises a colloidal metal; and
ii) detecting the colloidal detection reagent.

78. The method of claim 77, wherein detecting the colloidal detection reagent in step (d)(ii) comprises detection of the colloidal metal.

79. The method of claim 77, wherein detecting the colloidal detection reagent in step (d)(ii) comprises:
a) applying a developing reagent to the solid support, wherein the developing agent is suitable for forming a precipitate in the presence of the colloidal metal; and
b) detecting the colloidal detection reagent by detecting the formation of the precipitate at the solid support.

80. The method of claim 79, wherein the formation of the precipitate is detected by visual, electronic, or magnetic detection.

81. The method of claim 79 or claim 80, wherein the formation of the precipitate is detected by a mechanical reader.

82. The method of any one of claims 79-81, wherein the developing reagent comprises silver.

83. The method of any one of claims 77-82, wherein the label comprises biotin or a derivative thereof, and wherein the first moiety of the colloidal detection reagent comprises neutravidin, streptavidin, or an antigen-binding domain that specifically binds biotin.

84. The method of claim 83, wherein the first moiety of the colloidal detection reagent comprises neutravidin, and wherein the second moiety of the colloidal detection reagent comprises a colloidal gold ion.

85. The method of any one of claims 75-84, wherein the conditions in step (b) are suitable for amplification by polymerase chain reaction (PCR).

86. The method of any one of claims 75-84, wherein the conditions in step (b) are suitable for amplification by recombinase-polymerase assay (RPA), nucleic acid sequenced-based chain assay (NASBA), rolling circle amplification, branched chain amplification, ligation amplification, or loop-mediated isothermal amplification.

87. The method of any one of claims 75-86, wherein the portion of the sample in admixture with the PCR amplification mixture is passed through the continuous capillary tubing using a peristaltic pump, high performance liquid chromatography (HPLC) pump, precision syringe pump, or vacuum.

88. The method of any one of claims 75-87, further comprising, prior to step (b): passing the portion of the sample in admixture with the amplification mixture through a preheating zone at between about 20° C. and about 55° C. via the continuous capillary tubing.

89. The method of claim 88, wherein the preheating zone is between about 37° C. and about 42° C.

90. The method of claim 88 or claim 89, wherein the portion of the sample in admixture with the amplification mixture is passed through the preheating zone for up to 30 minutes.

91. The method of claim 90, wherein the portion of the sample in admixture with the amplification mixture is passed through the preheating zone for about 15 minutes.

92. The method of any one of claims 75-91, further comprising, prior to step (b): passing the portion of the sample in admixture with the amplification mixture through an activation zone at between about 80° C. and about 100° C. via the continuous capillary tubing.

93. The method of claim 92, wherein the activation zone is between about 90° C. and about 95° C.

94. The method of claim 92 or claim 93, wherein the portion of the sample in admixture with the amplification mixture is passed through the activation zone for up to 20 minutes.

95. The method of claim 94, wherein the portion of the sample in admixture with the amplification mixture is passed through the activation zone for between about 5 minutes and about 10 minutes.

96. The method of any one of claims 75-95, further comprising, after step (b) and prior to step (c): passing the portion of the sample in admixture with the amplification mixture through an extension zone at between about 55° C. and about 72° C. via the continuous capillary tubing.

97. The method of any one of claims 75-96, further comprising, after step (b) and prior to step (c):
i) mixing at least a portion of a second sample with an amplification mixture comprising deoxyribonucleotides, a polymerase, and a second primer pair, wherein the second primer pair comprises a third primer comprising a label and a fourth oligonucleotide sequence that hybridizes with a first strand of a portion of a second nucleic acid, and a fourth primer comprising a fifth oligonucleotide sequence that hybridizes with a second strand of the portion of the second nucleic acid opposite the first strand and a third capture moiety;
ii) passing the portion of the second sample in admixture with the amplification mixture through the first, second, and third stationary temperature zones for a second plurality of cycles through the continuous capillary tubing under conditions suitable for amplification of the portion of the second nucleic acid, if present in the sample, wherein each cycle of the second plurality comprises:
1) passing the portion of the second sample in admixture with the amplification mixture through the first stationary temperature zone via the continuous capillary tubing at the first temperature and for the first duration suitable for denaturing the strands of the second nucleic acid, if present in the second sample,
2) after step (ii)(1), passing the portion of the second sample in admixture with the amplification mixture through the second stationary temperature zone via the continuous capillary tubing at the second temperature and for the second duration suitable for annealing the third and fourth primers to the respective strands of the second nucleic acid, if present in the second sample, and
3) after step (ii)(2), passing the portion of the second sample in admixture with the amplification mixture through the third stationary temperature zone via the continuous capillary tubing at the third temperature and for the third duration suitable for amplifying the second nucleic acid, if present in the second sample, via the polymerase and second primer pair;
wherein the second nucleic acid, if present in the second sample, is associated concurrently with the amplified first nucleic acid target, if present in the first sample, with a fourth capture moiety that associates with the third capture moiety, wherein the fourth capture moiety is coupled to a solid support; and
wherein the association of the amplified second nucleic acid, if present in the second sample, with the solid support is detected concurrently with the hybridization of the amplified first nucleic acid, if present in the first sample, and wherein association of the amplified second nucleic acid target with the solid support indicates the presence of the second nucleic acid target in the second sample.

98. The method of claim 97, wherein the first and the second samples are the same.

99. The method of claim 97 or claim 98, wherein the first and the second nucleic acids are different.

100. The method of any one of claims 97-99, further comprising, after passing the portion of the first sample in admixture with the amplification mixture through the first, second, and third stationary temperature zones for the plurality of cycles, and prior to passing the portion of the second sample in admixture with the amplification mixture through the first, second, and third stationary temperature zones for the second plurality of cycles:
passing a volume of air through the continuous capillary tubing sufficient to separate the portion of the first sample in admixture with the amplification mixture and the portion of the second sample in admixture with the amplification mixture.

101. The method of claim 100, further comprising, after passing the volume of air through the continuous capillary tubing, and prior to passing the portion of the second sample in admixture with the amplification mixture through the first, second, and third stationary temperature zones for the second plurality of cycles:
passing a solution comprising sodium hypochlorite at a concentration of between about 0.1% and about 10% through the continuous capillary tubing.

102. The method of claim 101, wherein the solution comprises sodium hypochlorite at a concentration of about 1.6%.

103. The method of claim 101 or claim 102, further comprising, after passing the bleach solution through the continuous capillary tubing, and prior to passing the portion of the second sample in admixture with the amplification mixture through the first, second, and third stationary temperature zones for the second plurality of cycles:
passing a solution comprising thiosulfate at a concentration of between about 5 mM and about 500 mM through the continuous capillary tubing.

104. The method of claim 103, wherein the solution comprises thiosulfate at a concentration of about 20 mM.

105. The method of claim 103 or claim 104, further comprising, after passing the thiosulfate solution through the continuous capillary tubing, and prior to passing the portion of the second sample in admixture with the amplification mixture through the first, second, and third stationary temperature zones for the second plurality of cycles:
passing water through the continuous capillary tubing.

106. The method of claim 105, further comprising, after passing the water through the continuous capillary tubing, and prior to passing the portion of the second sample in admixture with the PCR amplification mixture through the first, second, and third stationary temperature zones for the second plurality of cycles:
passing a volume of air through the continuous capillary tubing sufficient to separate the water and the portion of the second sample in admixture with the PCR amplification mixture.

107. The method of any one of claims 75-106, wherein step (a) comprises inserting the portion of the sample into the continuous capillary tubing and mixing the portion of the sample with the amplification mixture using a robotic arm or valve system.

108. The method of any one of claims 75-107, wherein the nucleic acid comprises DNA.

109. The method of any one of claims 75-107, wherein the nucleic acid comprises RNA.

110. The method of claim 109, further comprising, prior to step (a):
incubating at least a portion of the sample with a reverse transcriptase, primers, and deoxyribonucleotides under conditions suitable for generation of a cDNA synthesized from the RNA, wherein the cDNA is mixed with the amplification mixture in step (a).

111. The method of claim 110, wherein the primers used prior to step (a) are random primers, poly-dT primers, or primers specific for the portion of the RNA.

112. The method of claim 110 or claim 111, wherein the portion of the sample is incubated with the reverse transcriptase, primers, and deoxyribonucleotides while being passed through a cDNA synthesis zone between about 37° C. and about 42° C. via the continuous capillary tubing for a time sufficient for generation of a cDNA synthesized from the RNA.

113. The method of claim 112, further comprising, after passing the portion of the sample in admixture with the reverse transcriptase, primers, and deoxyribonucleotides through the cDNA synthesis zone, and prior to step (b):
passing the portion of the sample in admixture with the reverse transcriptase, primers, and deoxyribonucleotides through an activation zone at about 95° C. via the continuous capillary tubing.

114. The method of any one of claims 75-113, wherein, during each cycle of the plurality, the portion of the sample in admixture with the amplification mixture is passed through the first stationary temperature zone at between about 80° C. and about 100° C. for 1 second to about 10 minutes.

115. The method of any one of claims 75-114, wherein, during each cycle of the plurality, the portion of the sample in admixture with the amplification mixture is passed through the second stationary temperature zone between about 45° C. and about 65° C. for 2 seconds to about 60 seconds.

116. The method of any one of claims 75-115, wherein, during each cycle of the plurality, the portion of the sample in admixture with the amplification mixture is passed through the third stationary temperature zone at between about 57° C. and about 74° C. for 3 seconds to about 60 seconds.

117. The method of any one of claims 75-114, wherein, during each cycle of the plurality, the portion of the sample in admixture with the PCR amplification mixture is passed through both the second stationary temperature zone and the third stationary temperature zone at between about 45° C. and about 80° C. for between about 0.5 seconds and about 5 minutes.

118. The method of any one of claims 75-117, wherein the plurality of cycles comprises greater than or equal to 2 cycles and less than or equal to 100 cycles.

119. The method of any one of claims 75-118, further comprising, prior to step (a), incubating the portion of the sample with a lysis buffer comprising greater than or equal to 0.1% and less than or equal to 10% N,N-dimethyl-N-dodecylglycine betaine (w/v).

120. The method of any one of claims 75-119, wherein the sample is further mixed in step (a) with betaine.

121. The method of any one of claims 75-120, wherein the sample is further mixed in step (a) with a fluorescent or colored dye.

122. The method of any one of claims 76-121, wherein the second primer comprises:
the second oligonucleotide sequence, wherein the second oligonucleotide sequence allows for primer extension in the 5′ to 3′ direction; and
the third oligonucleotide sequence, wherein the third oligonucleotide sequence is oriented in the opposite 5′ to 3′ direction compared with the direction of primer extension from the second oligonucleotide sequence.

123. The method of claim 122, wherein the third oligonucleotide sequence comprises a modified nucleotide at the 3′ terminus that blocks primer extension.

124. The method of claim 122 or claim 123, wherein the second primer further comprises one or more linkers between the 5′ end of the third oligonucleotide sequence and the 5′ end of the second oligonucleotide sequence.

125. The method of any one of claims 75-124, wherein the first capture moiety is affixed to a spacer reagent and, wherein the spacer reagent is coupled to the solid support.

126. The method of claim 125, wherein the spacer reagent comprises a serum albumin protein.

127. The method of claim 125, wherein the spacer reagent comprises a dendrimer.

128. The method of any one of claims 75-127, wherein the sample comprises whole blood, serum, saliva, urine, soil, tissue, or an environmental sample.

129. The method of any one of claims 75-128, wherein the nucleic acid comprises a viral nucleic acid.

130. The method of claim 129, wherein the viral nucleic acid is from a virus selected from the group consisting of HIV, HBV, HCV, West Nile, Zika, and parvovirus.

131. The method of any one of claims 75-128, wherein the nucleic acid comprises a bacterial, archaean, protozoan, fungal, plant, or animal nucleic acid.

132. An apparatus for amplifying a nucleic acid from a sample, the apparatus comprising:
capillary tubing arranged around a support in a plurality of circuits, wherein each circuit of the plurality comprises a first, a second, and a third stationary temperature zone, and wherein the capillary tubing is heated to a first temperature in the first stationary temperature zone, a second temperature in the second stationary temperature zone, and a third temperature in the third stationary temperature zone;
a robotic arm configured to introduce into the capillary tubing a sample comprising a nucleic acid in admixture with an amplification mixture comprising deoxyribonucleotides, a polymerase, and a primer pair; and
a pump or vacuum configured to pass the sample comprising the nucleic acid in admixture with the amplification mixture through the plurality of circuits within the capillary tubing.

133. The apparatus of claim 132, further comprising one or more processors, a memory, one or more programs, wherein the one or more programs are stored in the memory and configured to be executed by the one or more processors, the one or more programs including instructions for controlling the temperature of the first, second, and third stationary temperature zones.

134. The apparatus of claim 132 or claim 133, further comprising:
an incubator for a cDNA synthesis zone in which the capillary tubing is heated to between about 37° C. and about 42° C. upstream of the plurality of circuits.

135. The apparatus of any one of claims 132-134, further comprising:
an incubator for an activation zone in which the capillary tubing is heated to about 95° C. upstream of the plurality of circuits.

136. The apparatus of any one of claims 132-135, wherein the capillary tubing forms a conical, cylindrical, or spiral shape in each circuit of the plurality.

137. The apparatus of any one of claims 132-136, wherein the capillary tubing comprises polytetrafluoroethylene (PTFE).

138. The apparatus of any one of claims 132-137, wherein the plurality of circuits of the capillary tubing comprise from about 25 to about 44 circuits.

139. The apparatus of any one of claims 132-138, wherein the robotic arm comprises a peristaltic or HPLC pump configured to introduce the sample comprising the nucleic acid target in admixture with an amplification mixture into the capillary tubing, and wherein the apparatus further comprises a secondary pump configured to pull the sample comprising the nucleic acid target in admixture with an amplification mixture through the capillary tubing.

140. The apparatus of any one of claims 132-139, further comprising:
an incubator for a PCR extension zone in which the capillary tubing is heated to between about 55° C. and about 72° C. downstream of the plurality of circuits.

141. The apparatus of any one of claims 132-140, wherein the vacuum configured to pass the sample comprising the nucleic acid in admixture with the amplification mixture through the plurality of circuits is a peristaltic pump, high performance liquid chromatography (HPLC) pump, or precision syringe pump.

142. A method for detecting an antigen in a sample, the method comprising:
a) providing a plurality of single-stranded oligonucleotide capture sequences each affixed to a solid support;
b) after step (a), contacting the solid supports with an antigen-binding domain that specifically binds an antigen, wherein the antigen-binding domain is coupled to a single-stranded oligonucleotide sequence that hybridizes with at least one of the single-stranded oligonucleotide capture sequences on the solid supports, and wherein the microarray is contacted with the antigen-binding domain under conditions suitable for the single-stranded oligonucleotide sequence of the antigen binding domain to hybridize with the at least one single-stranded oligonucleotide capture sequence on the solid supports;
c) after step (a), contacting the solid supports with at least a portion of the sample under conditions suitable for the antigen-binding domain to bind the antigen, if present in the sample;
d) after step (a), applying a colloidal detection reagent to the solid supports, wherein the colloidal detection reagent comprises a first moiety that specifically binds to the antigen if present and a second moiety that comprises a colloidal metal;
e) after (d), washing the solid supports with a wash solution; and
f) after steps (a)-(e), detecting the colloidal detection reagent, wherein detection of the colloidal detection reagent indicates the presence of the antigen in the sample.

143. The method of claim 142, wherein the solid supports are arranged as a microarray, a multiplex bead array, or a well array.

144. The method of claim 142, wherein the solid supports are nitrocellulose, silica, plastic, or hydrogel.

145. The method of claim 142, wherein detecting the colloidal detection reagent in step (f) comprises detection of the colloidal metal.

146. The method of claim 142, wherein detecting the colloidal detection reagent in step (f) comprises:
1) applying a developing reagent to the solid supports, wherein the developing agent is suitable for forming a precipitate in the presence of the colloidal metal; and
2) detecting the colloidal detection reagent by detecting the formation of the precipitate.

147. The method of claim 146, wherein the formation of the precipitate is detected by visual, electronic, or magnetic detection.

148. The method of claim 146 or claim 147, wherein the formation of the precipitate is detected by a mechanical reader.

149. The method of any one of claims 146-148, wherein the developing reagent comprises silver.

150. The method of any one of claims 142-149, wherein the first moiety comprises a second antigen binding domain that specifically binds to the antigen, wherein the second antigen binding domain is coupled to biotin or a derivative thereof, and wherein the colloidal suspension is coupled to avidin, neutravidin, streptavidin, or a derivative thereof bound to the biotin.

151. The method of any one of claims 142-150, wherein the colloidal metal is gold, platinum, palladium, or ruthenium.

152. The method of any one of claims 142-151, wherein the single-stranded oligonucleotide capture sequence at each spot of the plurality is coupled to a spacer reagent, and the spacer reagent is coupled to the solid supports.

153. The method of claim 152, wherein the spacer reagent comprises a serum albumin protein.

154. The method of claim 152, wherein the spacer reagent comprises a dendrimer.

155. The method of any one of claims 142-154, further comprising, prior to step (c), exposing the sample to a lysis buffer comprising greater than or equal to 0.1% and less than or equal to 10% N,N-dimethyl-N-dodecylglycine betaine (w/v).

156. The method of claim 155, wherein the lysis buffer comprises greater than or equal to 0.5% and less than or equal to 4% N,N-dimethyl-N-dodecylglycine betaine (w/v).

157. The method of claim 155, wherein the lysis buffer comprises greater than or equal to 1% and less than or equal to 2% N,N-dimethyl-N-dodecylglycine betaine (w/v).

158. The method of any one of claims 155-157, wherein the sample is exposed to the lysis buffer at a ratio between 1:50 sample:lysis buffer and 50:1 sample:lysis.

159. The method of claim 158, wherein the portion of the sample is exposed to the lysis buffer at a ratio of about 1:1 sample:lysis buffer.

160. The method of any one of claims 155-159, wherein the lysis buffer further comprises 0.1× to 5× phosphate buffered saline (PBS) buffer or Tris EDTA (TE) buffer.

161. The method of claim 160, wherein the lysis buffer further comprises 1×PBS.

162. The method of any one of claims 142-161, wherein the solid supports are contacted with the antigen-binding domain in step (b) in the presence of a hybridization buffer comprising 0.1× to 10× saline sodium citrate (SSC) buffer, 0.001% to 30% blocking agent, and 0.01% to 30% crowding agent.

163. The method of claim 162, wherein the blocking agent comprises bovine serum albumin (BSA), polyethylene glycol (PEG), casein, or polyvinyl alcohol (PVA).

164. The method of claim 163, wherein the blocking agent comprises BSA, and the BSA is present in the buffer at 1% to 3%.

165. The method of any one of claims 162-164, wherein the crowding agent is Polyethylene Glycol Bisphenol A Epichlorohydrin Copolymer.

166. The method of claim 165, wherein the Polyethylene Glycol Bisphenol A Epichlorohydrin Copolymer is present in the hybridization buffer at 1% to 3%.

167. The method of any one of claims 162-166, wherein the buffer comprises 2× to 5×SSC buffer.

168. The method of any one of claims 142-167, further comprising, prior to steps (b) and (c), blocking the solid supports using a solution comprising BSA.

169. The method of claim 168, wherein the solid supports are blocked for 1 hour at 37° C. using 2% BSA solution.

170. The method of claim 168 or claim 169, further comprising washing the solid supports with a wash solution after blocking the solid supports.

171. The method of any one of claims 142-170, further comprising, after steps (b) and (c) and prior to step (d), washing the solid supports with a wash buffer comprising 0.1× to 10×SSC buffer and 0.01% to 30% detergent.

172. The method of claim 171, wherein the detergent comprises 0.05% to 5% N-lauroylsarcosine sodium salt.

173. The method of claim 171 or claim 172, wherein the wash buffer comprises 1× to 5×SSC buffer.

174. The method of any one of claims 155-173, wherein one or more of the lysis buffer, wash buffer, and hybridization buffer further comprises a control oligonucleotide that hybridizes with at least one of the single-stranded oligonucleotide capture sequences on its solid support.

175. The method of any one of claims 142-174, wherein the antigen is a viral antigen.

176. The method of claim 175, wherein the viral antigen is from a virus selected from the group consisting of: HIV, HBV, HCV, West Nile, Zika, and parvovirus.

177. The method of any one of claims 142-174, wherein the antigen is a bacterial, archaean, protozoan, fungal, plant, or animal antigen.

178. The method of any one of claims 1-177, wherein the sample comprises whole blood, serum, saliva, urine, soil, tissue, or an environmental sample.

179. A kit, comprising:
a) a plurality of single-stranded oligonucleotide capture sequences each affixed to a solid support; and
b) a plurality of antigen-binding domains, wherein each antigen-binding domain of the plurality specifically binds an antigen, and wherein each antigen-binding domain of the plurality is coupled to a single-stranded oligonucleotide sequence that is substantially complementary to a single-stranded oligonucleotide sequence affixed to the solid supports.

180. The kit of claim 179, further comprising:
c) a second antigen-binding domain coupled to a colloidal detection reagent, wherein the second antigen-binding domain specifically binds an antigen that is also specifically bound by an antigen-binding domain of the plurality of antigen-binding domains in (b).

181. A plurality of single-stranded oligonucleotide capture sequences each affixed to a solid support, wherein each single-stranded oligonucleotide capture sequence is independently selected from the group consisting of SEQ ID NOs:1-15.

182. The plurality of sequences of claim 181, wherein the single-stranded oligonucleotide capture sequence at each solid support is coupled to a spacer reagent, and the spacer reagent is coupled to the solid supports.

183. The plurality of sequences of claim 182, wherein the spacer reagent comprises a serum albumin protein.

184. The plurality of sequences of claim 182, wherein the spacer reagent comprises a dendrimer.

185. A kit, comprising:
a) the plurality of any one of claims 181-184; and
b) a plurality of antigen binding domains, wherein each antigen binding domain of the plurality is coupled to a single-stranded oligonucleotide sequence independently selected from the group consisting of SEQ ID NOs:16-30.

186. A kit, comprising:
a) the plurality of any one of claims 181-184; and
b) a plurality of primer pairs, wherein each primer pair of the plurality comprises:
1) a first primer comprising a label and a first oligonucleotide sequence that hybridizes with a first strand of a nucleic acid; and
2) a second primer comprising a second oligonucleotide sequence that hybridizes with a second strand of the portion of the nucleic acid opposite the first strand and a third oligonucleotide sequence, wherein the third oligonucleotide sequence of each first primer is independently selected from the group consisting of SEQ ID NOs:16-30.

187. A plurality of single-stranded oligonucleotide capture sequences each affixed to a solid support, wherein each single-stranded oligonucleotide capture sequence is independently selected from the group consisting of SEQ ID NOs:16-30.

188. The plurality of sequences of claim 187, wherein the single-stranded oligonucleotide capture sequence at each solid support is coupled to a spacer reagent, and the spacer reagent is coupled to the solid supports.

189. The plurality of sequences of claim 188, wherein the spacer reagent comprises a serum albumin protein.

190. The plurality of sequences of claim 188, wherein the spacer reagent comprises a dendrimer.

191. A kit, comprising:
a) the plurality of sequences of any one of claims 187-190; and
b) a plurality of antigen binding domains, wherein each antigen binding domain of the plurality is coupled to a single-stranded oligonucleotide sequence independently selected from the group consisting of SEQ ID NOs:1-15.

192. A kit, comprising:
a) the plurality of sequences of any one of claims 187-190; and
b) a plurality of primer pairs, wherein each primer pair of the plurality comprises:
1) a first primer comprising a label and a first oligonucleotide sequence that hybridizes with a first strand of a nucleic acid; and
2) a second primer comprising a second oligonucleotide sequence that hybridizes with a second strand of the portion of the nucleic acid opposite the first strand and a third oligonucleotide sequence, wherein the third oligonucleotide sequence of each first primer is independently selected from the group consisting of SEQ ID NOs:1-15.

193. The plurality of sequences of any one of claims 181-184 and 187-190, wherein the solid supports are arranged as a microarray, a multiplex bead array, or a well array.

194. The plurality of sequences of any one of claims 181-184 and 187-190, wherein the solid supports are nitrocellulose, silica, plastic, or hydrogel.

195. The kit of any one of claims 179, 180, 185, 186, 191, and 192, wherein the solid supports are arranged as a microarray, a multiplex bead array, or a well array.

196. The kit of any one of claims 179, 180, 185, 186, 191, and 192, wherein the solid supports are nitrocellulose, silica, plastic, or hydrogel.
